(19) United States
(12) Patent Application Publication
(10) Pub. No.: US 2024/0043419 A1
(43) Pub. Date: Feb. 8, 2024

(54) CLASS OF 1,7-NAPHTHYRIDINE COMPOUNDS AND APPLICATION THEREOF

(71) Applicant: Medshine Discovery Inc., Nanjing, Jiangsu (CN)

(72) Inventors: Shaohui Wang, Shanghai (CN); Chundao Yang, Shanghai (CN); Shuhui Chen, Shanghai (CN)

(73) Assignee: Medshine Discovery Inc., Nanjing, Jiangsu (CN)

(21) Appl. No.: 18/028,632

(22) PCT Filed: Sep. 27, 2021
(86) PCT No.: PCT/CN2021/121067

§ 371 Date: Mar. 27, 2023

(30) Foreign Application Priority Data
Sep. 27, 2020 (CN) 202011032189.4
Aug. 26, 2021 (CN) 202110990744.2

(51) Int. Cl. C07D 471/04; C07D 519/00; A61P 35/00

(52) U.S. Cl. CPC C07D 471/04; C07D 519/00; A61P 35/00

(57) ABSTRACT

A class of 1,7-naphthyridine compounds and an application thereof, the compounds being compounds represented by formula (II) or a pharmaceutically acceptable salt thereof.

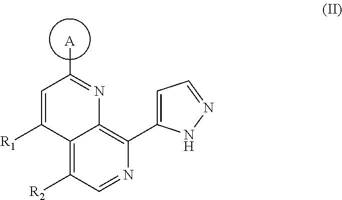

### REFERENCE TO RELATED APPLICATIONS

This application claims the priority of
CN202011032189.4, filed on Sep. 27, 2020; and
CN202110990744.2, filed on Aug. 26, 2021.

### FIELD OF THE INVENTION

The present disclosure relates to a class of 1,7-naphthyridine compounds and use thereof, in particular to use of the class of compounds or pharmaceutically acceptable salts thereof in the manufacture of the treatment of related diseases.

### BACKGROUND OF THE INVENTION

Ataxia telangiectasia and Rad3-related kinase (ATR) is a member of the family of phosphatidylinositol 3-kinase-related kinases (PIKK). It consists of 2644 amino acids, with an N-terminal ATR-interacting-protein (ATRIP) binding domain, which is an important domain for ATR activation, and a C-terminal kinase domain for downstream protein phosphorylation.

ATR is a key protein in the DNA damage repair signaling pathway, which has functions of regulating cell cycle, promoting DNA damage repair, stabilizing replication fork structure, limiting replication initiation and relieving replication stress, etc.

DNA replication needs to be completed before cells enter M phase. DNA is often mutated or damaged due to the interference of various endogenous and exogenous factors. For example, free radicals generated during metabolism in the body, spontaneous errors in DNA replication and recombination, UV and ionizing radiation (IR) in the environment, and some chemicals can cause DNA damage. The abnormal DNA must be repaired, otherwise it will trigger mitotic catastrophe and cause cell death. G1 checkpoint and G2 checkpoint are two main cell cycle checkpoints, and they are jointly responsible for the recognition and repair of DNA damage. Nearly 70% of cancerous cells have defects in the tumor suppressor gene p53, which make them lose the G1 checkpoint function and rely more on the G2 checkpoint to complete DNA repair. ATR kinase is a protein that plays a critical role at the G2 checkpoint. After ATR detects DNA damage, it activates the downstream CHK1, and CHK1 inhibits the downstream CDC25, thereby causing the arrest of G2 phase and helping the damaged DNA to be repaired.

Inhibition of ATR kinase is expected to abolish the G2 phase arrest and push cancer cells to enter mitosis prematurely, eventually leading to cancer cell apoptosis, while normal cells can use the G1 checkpoint to complete the repair of damaged DNA. ATR kinase inhibitors affect cancer cells with genetic defects more than normal cells. ATR is a very potential anti-tumor target, and also a research hotspot in the field of anti-tumor in recent years. At present, a variety of small molecule inhibitors of ATR such as berzosertib (VX-970), ceralasertib (AZD-6738), BAY1895344 and M-4344 have entered a clinical trial stage.

### SUMMARY OF THE INVENTION

The present disclosure provides a compound of formula (II) or a pharmaceutically acceptable salt thereof,

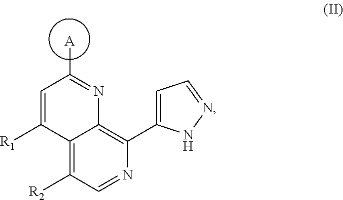

wherein
Ring A is selected from

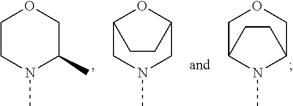

R₁ is H, D, F, Cl, Br, I, CN, C₁₋₃ alkyl, C₁₋₃ alkoxy, C₃₋₆ cycloalkyl, 5- to 10-membered heterocycloalkyl, 5- to 10-membered heteroaryl, phenyl, 5- to 6-membered heterocycloalkenyl, —C(═O)R₃, —C(═O)OR₃, —C(═O)NR₄R₅ or —NR₆C(═O)R₇, wherein the C₁₋₃ alkyl, C₁₋₃ alkoxy, C₃₋₆ cycloalkyl, 5- to 10-membered heterocycloalkyl, 5- to 10-membered heteroaryl, phenyl and 5- to 6-membered heterocycloalkenyl are optionally substituted with 1, 2 or 3 Rₐ;
R₂ is F, Cl, Br, I, OH, NH₂, CN or COOH;
R₃ is independently selected from H, D, C₁₋₃ alkyl, C₁₋₃ alkoxy and C₃₋₆ cycloalkyl;
R₄ and R₅ are independently selected from H, D, C₁₋₃ alkyl, C₁₋₃ alkoxy, and C₃₋₆ cycloalkyl, or R₄ and R₅ are taken together with the N atom to which they are attached to form 5- to 6-membered heterocycloalkyl, wherein the 5- to 6-membered heterocycloalkyl is optionally substituted with 1, 2 or 3 Rₐ;
R₆ and R₇ are independently selected from H, D, C₁₋₃ alkyl, C₁₋₃ alkoxy, and C₃₋₆ cycloalkyl, or R₆ and R₇ are taken together with the —N(C═O)— to which they are attached to form 5- to 10-membered heterocycloalkyl, wherein the 5- to 10-membered heterocycloalkyl is optionally substituted with 1, 2 or 3 Rₐ;
Rₐ is H, D, F, Cl, Br, I, OH, NH₂, CN, COOH, —SO₂C₁₋₃ alkyl, C₁₋₃ alkyl or C₁₋₃ alkoxy.

In some embodiments of the present disclosure, Rₐ is independently selected from H, D, F, OH, CN, —OCH₃, —CH₃ and —SO₂CH₃, and other variables are as defined in the present disclosure.

In some embodiments of the present disclosure, R₂ is F, and other variables are as defined in the present disclosure.

In some embodiments of the present disclosure, R₁ is selected from K₁

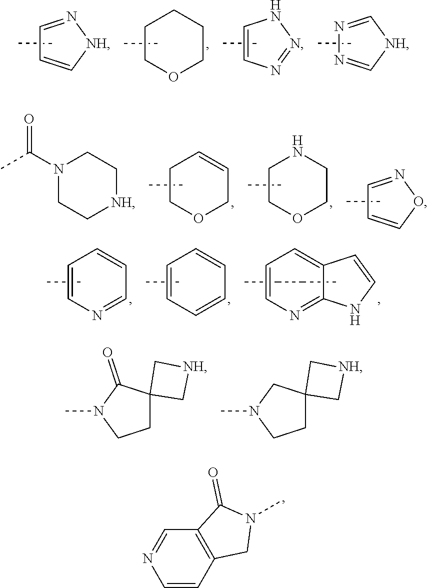

—OC₁₋₃ alkyl and C₃₋₆ cycloalkyl, wherein the

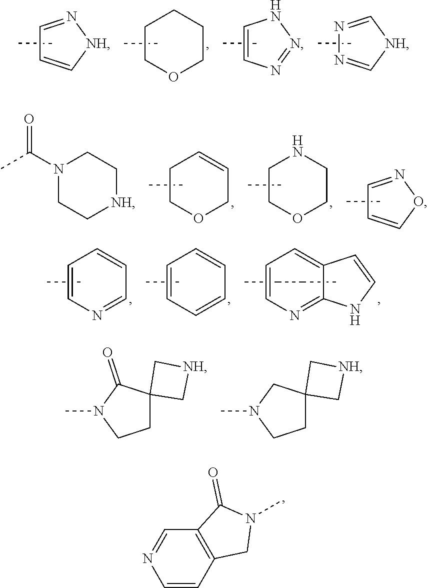

—OC₁₋₃ alkyl and C₃₋₆ cycloalkyl are optionally substituted with 1, 2 or 3 Rₐ, and other variables are as defined in the present disclosure.

In some embodiments of the present disclosure, R₁ is selected from

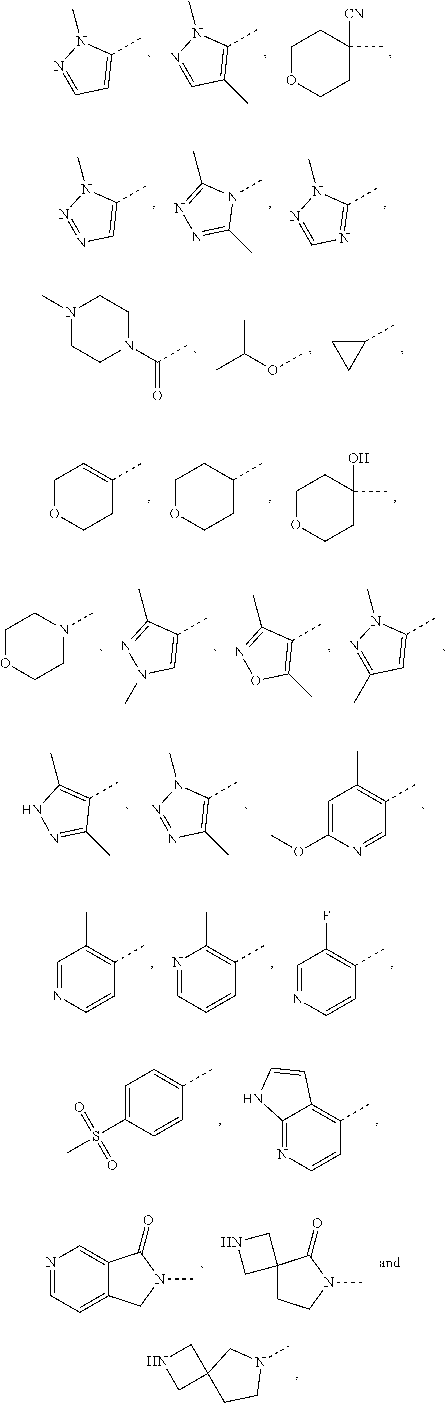

and other variables are as defined in the present disclosure.

In some embodiments of the present disclosure, R₁ is selected from

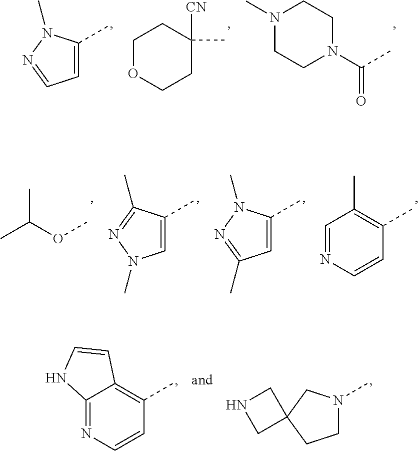

and other variables are as defined in the present disclosure.

In some embodiments of the present disclosure, the compound is as shown in formulae (II-1), (II-2) and (II-3),

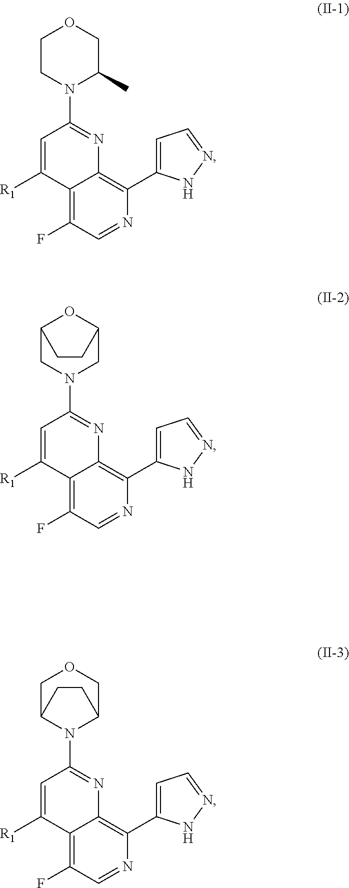

and other variables are as defined in the present disclosure.

The present disclosure also provides a compound of formula (I) or a pharmaceutically acceptable salt thereof,

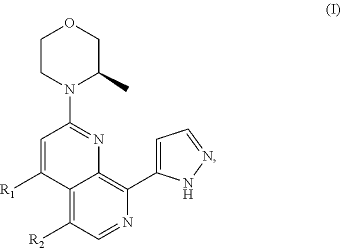

wherein
R₁ is

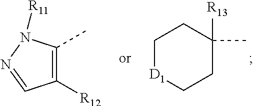

D₁ is 0 or S;
R₁₁ and R₁₂ are each independently H or C₁₋₃ alkyl;
R₁₃ is H, F, Cl, Br, I, OH, NH₂, CN, COOH or C₁₋₃ alkyl;
R₂ is H, F, Cl, Br, I, OH, NH₂, CN, COOH or C₁₋₃ alkyl.

In some embodiments of the present disclosure, the above R₁₁ and R₁₂ independently H or CH₃, and other variables are as defined in the present disclosure.

In some embodiments of the present disclosure, the above R₁₃ is H, F, Cl, Br, I, OH, NH₂, CN or COOH, and other variables are as defined in the present disclosure.

In some embodiments of the present disclosure, the above R₂ is H, F, Cl, Br, I, OH or NH₂, and other variables are as defined in the present disclosure.

In some embodiments of the present disclosure, the above R₁ is

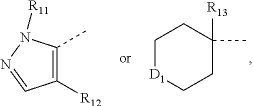

and other variables are as defined in the present disclosure.

In some embodiments of the present disclosure, the above R₁ is

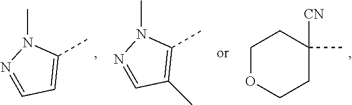

and other variables are as defined in the present disclosure.

The present disclosure also includes some embodiments obtained by any combination of the above variables.

In some embodiments of the present disclosure, the above compound is selected from

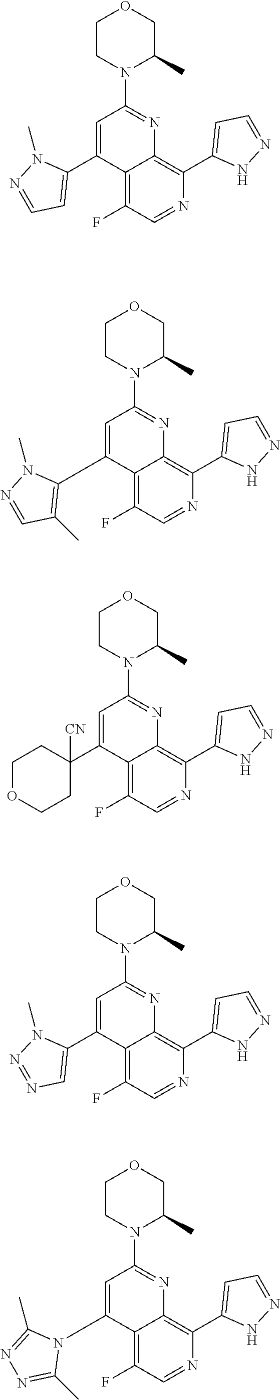 [SMILES: C[C@@H]1COCCN1c1cc(-c2ccnn2C)c2c(F)cnc(-c3ccn[nH]3)c2n1 ; Cc1cnn(C)c1-c1cc(N2CCOC[C@H]2C)nc2c(-c3ccn[nH]3)ncc(F)c12 ; C[C@@H]1COCCN1c1cc(C2(C#N)CCOCC2)c2c(F)cnc(-c3ccn[nH]3)c2n1 ; C[C@@H]1COCCN1c1cc(-c2cnnn2C)c2c(F)cnc(-c3ccn[nH]3)c2n1 ; Cc1nnc(C)n1-c1cc(N2CCOC[C@H]2C)nc2c(-c3ccn[nH]3)ncc(F)c12]

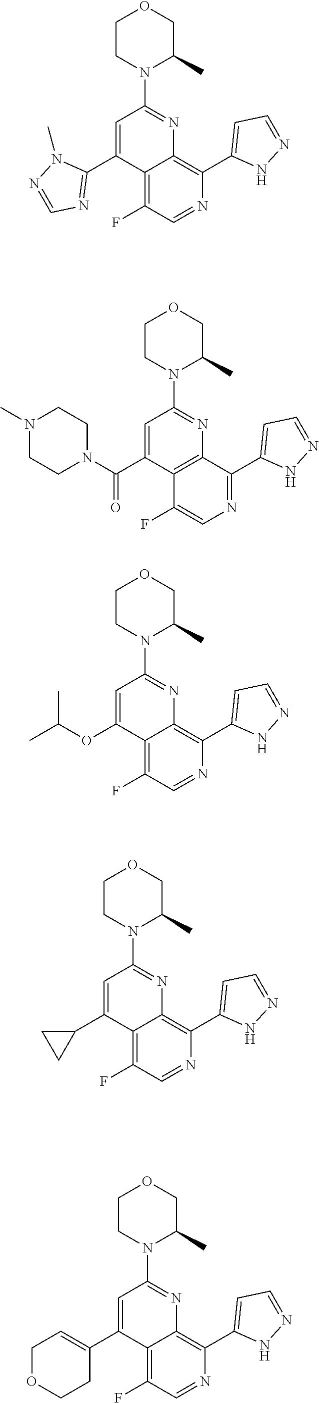 [SMILES: C[C@@H]1COCCN1c1cc(-c2ncnn2C)c2c(F)cnc(-c3ccn[nH]3)c2n1 ; C[C@@H]1COCCN1c1cc(C(=O)N2CCN(C)CC2)c2c(F)cnc(-c3ccn[nH]3)c2n1 ; CC(C)Oc1cc(N2CCOC[C@H]2C)nc2c(-c3ccn[nH]3)ncc(F)c12 ; C[C@@H]1COCCN1c1cc(C2CC2)c2c(F)cnc(-c3ccn[nH]3)c2n1 ; C[C@@H]1COCCN1c1cc(C2=CCOCC2)c2c(F)cnc(-c3ccn[nH]3)c2n1]

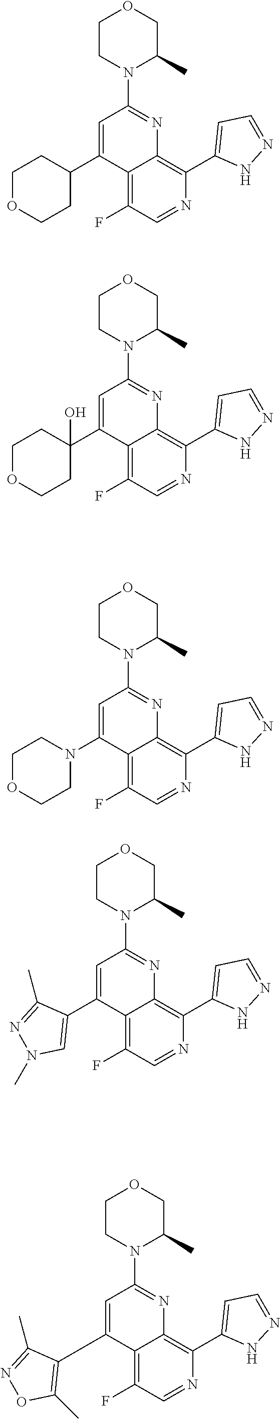 [SMILES: C[C@@H]1COCCN1c1cc(C2CCOCC2)c2c(F)cnc(-c3ccn[nH]3)c2n1 ; C[C@@H]1COCCN1c1cc(C2(O)CCOCC2)c2c(F)cnc(-c3ccn[nH]3)c2n1 ; C[C@@H]1COCCN1c1cc(N2CCOCC2)c2c(F)cnc(-c3ccn[nH]3)c2n1 ; Cc1nn(C)cc1-c1cc(N2CCOC[C@H]2C)nc2c(-c3ccn[nH]3)ncc(F)c12 ; Cc1noc(C)c1-c1cc(N2CCOC[C@H]2C)nc2c(-c3ccn[nH]3)ncc(F)c12]

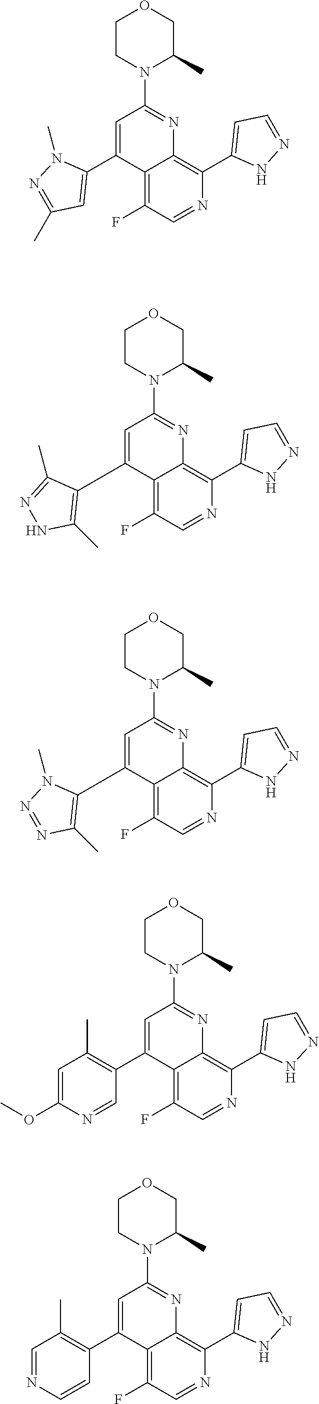 [SMILES: Cc1cc(-c2cc(N3CCOC[C@H]3C)nc3c(-c4ccn[nH]4)ncc(F)c23)n(C)n1 ; Cc1n[nH]c(C)c1-c1cc(N2CCOC[C@H]2C)nc2c(-c3ccn[nH]3)ncc(F)c12 ; Cc1nnn(C)c1-c1cc(N2CCOC[C@H]2C)nc2c(-c3ccn[nH]3)ncc(F)c12 ; COc1cc(C)c(-c2cc(N3CCOC[C@H]3C)nc3c(-c4ccn[nH]4)ncc(F)c23)cn1 ; Cc1cnccc1-c1cc(N2CCOC[C@H]2C)nc2c(-c3ccn[nH]3)ncc(F)c12]

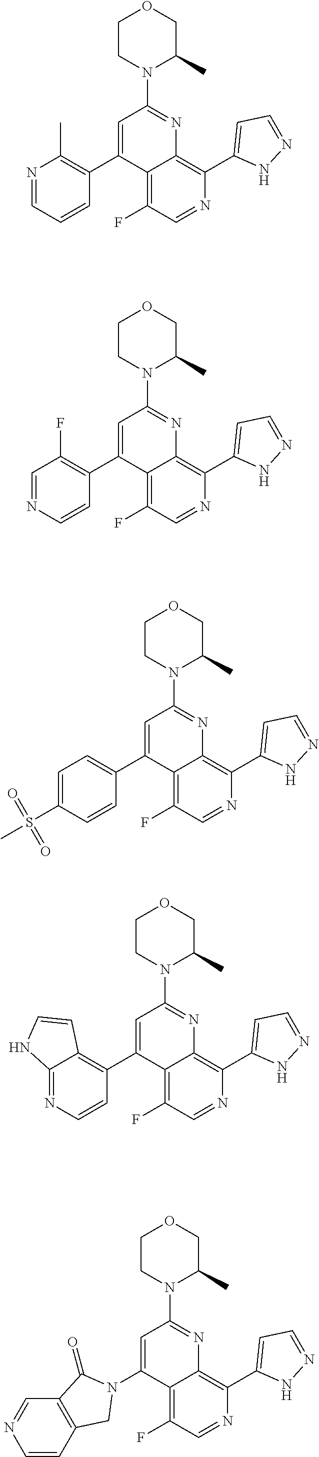 [SMILES: Cc1ncccc1-c1cc(N2CCOC[C@H]2C)nc2c(-c3ccn[nH]3)ncc(F)c12 ; C[C@@H]1COCCN1c1cc(-c2ccncc2F)c2c(F)cnc(-c3ccn[nH]3)c2n1 ; C[C@@H]1COCCN1c1cc(-c2ccc(S(C)(=O)=O)cc2)c2c(F)cnc(-c3ccn[nH]3)c2n1 ; C[C@@H]1COCCN1c1cc(-c2ccnc3[nH]ccc23)c2c(F)cnc(-c3ccn[nH]3)c2n1 ; C[C@@H]1COCCN1c1cc(N2Cc3ccncc3C2=O)c2c(F)cnc(-c3ccn[nH]3)c2n1]

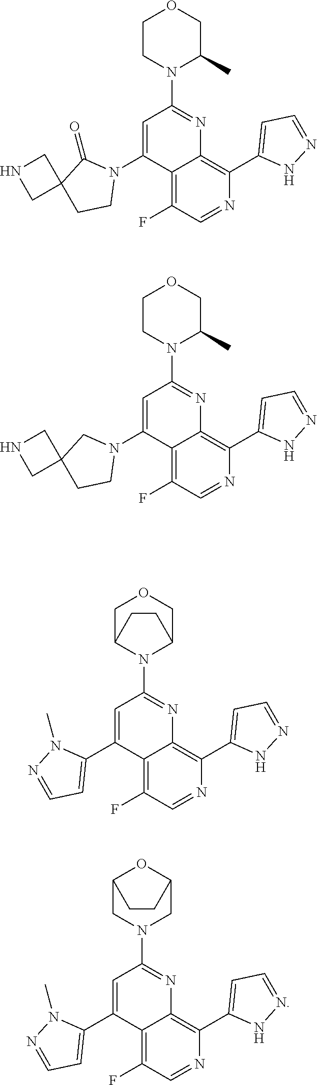 [SMILES: C[C@@H]1COCCN1c1cc(N2CCC3(CNC3)C2=O)c2c(F)cnc(-c3ccn[nH]3)c2n1 ; C[C@@H]1COCCN1c1cc(N2CCC3(CNC3)C2)c2c(F)cnc(-c3ccn[nH]3)c2n1 ; Cn1nccc1-c1cc(N2C3CCC2COC3)nc2c(-c3ccn[nH]3)ncc(F)c12 ; Cn1nccc1-c1cc(N2CC3CCC(C2)O3)nc2c(-c3ccn[nH]3)ncc(F)c12]

### Technical Effect

The compounds of the present disclosure have good inhibitory effects on LoVo tumor cells with mutations in the ATR signaling pathway; the compounds of the present disclosure have good inhibitory effects on the phosphorylation of CHK1 protein downstream of the ATR signaling pathway; the compounds of the present disclosure can improve multiple indicators of pharmacokinetics in mice, in which the in vivo clearance rate and half-life of intravenous injection and the maximum blood drug concentration and area under the drug-time curve of oral administration have significant advantages; and the compounds of the present disclosure can improve the inhibitory effects on tumor growth in mice.

### Definition and Term

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, allergic reaction or other problems or complications, commensurate with a reasonable benefit/risk ratio.

The term “pharmaceutically acceptable salt” means a salt of compounds disclosed herein that is prepared by reacting the compound having a specific substituent disclosed herein with a relatively non-toxic acid or base. When compounds disclosed herein contain a relatively acidic functional group, a base addition salt can be obtained by bringing the compound into contact with a sufficient amount of base in a pure solution or a suitable inert solvent. The pharmaceutically acceptable base addition salt includes a salt of sodium, potassium, calcium, ammonium, organic amine or magnesium or similar salts. When compounds disclosed herein contain a relatively basic functional group, an acid addition salt can be obtained by bringing the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, etc.; and an salt of amino acid (such as arginine and the like), and a salt of an organic acid such as glucuronic acid and the like. Certain specific compounds disclosed herein contain both basic and acidic functional groups and can be converted to any base or acid addition salt.

The pharmaceutically acceptable salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

Compounds disclosed herein may be present in a specific geometric or stereoisomeric form. The present disclosure contemplates all such compounds, including cis and trans isomers, (−)- and (+)-enantiomers, (R)- and (S)-enantiomers, diastereoisomer, (D)-isomer, (L)-isomer, and a racemic mixture and other mixtures, for example, a mixture enriched in enantiomer or diastereoisomer, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” means stereoisomers that are in a mirrored relationship with each other.

Unless otherwise specified, the term “cis-trans isomer” or “geometric isomer” is produced by the inability of a double bond or a single bond between ring-forming carbon atoms to rotate freely.

Unless otherwise specified, the term “diastereomer” refers to stereoisomers whose molecules have two or more chiral centers and are in a non-mirror-image relationship.

Unless otherwise specified, “(+)” means dextroisomer, “(−)” means levoisomer, and “(±)” means racemate.

Unless otherwise specified, a wedged solid bond ( [symbol] ) and a wedged dashed bond ( [symbol] ) indicate the absolute configuration of a stereocenter; a straight solid ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ); or a wavy line ( [symbol] ) indicates a straight solid bond ( [symbol] ) and a straight dashed bond ( [symbol] ).

Unless otherwise specified, the terms “tautomer” or “tautomeric form” means that different functional groups are in dynamic equilibrium at room temperature and can be rapidly converted into each other. If tautomers are possible (as in solution), a chemical equilibrium of tautomers can be achieved. For example, proton tautomers (also known as prototropic tautomers) include interconversions by proton transfer, such as keto-enol isomerization and imine-enamine isomerization. Valence tautomers include interconversions by recombination of some bonding electrons. A specific example of keto-enol tautomerization is interconversion between two tautomers pentane-2,4-dione and 4-hydroxypent-3-en-2-one.

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, 98% or more, 99% or more, 99.5% or more, 99.6% or more, 99.7% or more, 99.8% or more, or 99.9% or more.

Unless otherwise specified, the term “isomer excess” or “enantiomeric excess” means the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to afford the pure enantiomer. In addition, the enantiomer and the diastereoisomer are generally isolated through chromatography which uses a chiral stationary phase and optionally combines with a chemical derivative method (for example, carbamate generated from amine).

Compounds disclosed herein may contain an unnatural proportion of atomic isotopes at one or more of the atoms that make up the compounds. For example, a compound may be labeled with a radioisotope such as tritium (³H), iodine-125 (¹²⁵I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

Unless otherwise specified, D as described in the present disclosure refers to tritium (²H).

The term “optional” or “optionally” means that the subsequent event or condition may occur but not requisite, that the term includes the instance in which the event or condition occurs and the instance in which the event or condition does not occur.

The term “substituted” means that one or more than one hydrogen atoms on a specific atom are substituted by a substituent, including deuterium and hydrogen variants, as long as the valence of the specific atom is normal and the substituted compound is stable. When the substituent is oxo (i.e., ═O), it means two hydrogen atoms are substituted. Positions on an aromatic ring cannot be substituted by oxo.

The term “optionally substituted” means an atom can be substituted by a substituent or not, unless otherwise specified, the species and number of the substituent may be arbitrary so long as being chemically achievable.

When any variable (such as R) occurs in the constitution or structure of the compound more than once, the definition of the variable at each occurrence is independent. Thus, for example, if a group is substituted by 0-2 R, the group can be optionally substituted by up to two R, wherein the definition of R at each occurrence is independent. Moreover, a combination of the substituent and/or the variant thereof is allowed only when the combination results in a stable compound.

When a substituent is recited without specifying the atom through which it is connected to the substituted group, such substituent may be bonded through any of its atoms.

For example, pyridyl as a substituent can be connected to the substituted group through any carbon atom on the pyridine ring.

Unless otherwise specified, when a group has one or more connectable sites, any one or more sites of the group can be connected to other groups through chemical bonds. Where the connection position of the chemical bond is variable, and there is H atom(s) at a connectable site(s), when the connectable site(s) having H atom(s) is connected to the chemical bond, the number of H atom(s) at this site will correspondingly decrease as the number of the connected chemical bond increases, and the group will become a group of corresponding valence. The chemical bond between the site and other groups can be represented by a straight solid bond ( [symbol] ), a straight dashed bond ( [symbol] ), or a wavy line

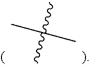

For example, the straight solid bond in —OCH₃ indicates that the group is connected to other groups through the oxygen atom in the group; the straight dashed bond in

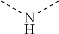

indicates that the group is connected to other groups through two ends of the nitrogen atom in the group; the wavy line in

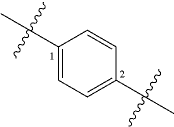 [SMILES: *c1ccc(*)cc1]

indicates that the group is connected to other groups through the 1- and 2-carbon atoms in the phenyl group;

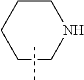

indicates that any connectable site on the piperidinyl group can be connected to other groups through one chemical bond, including at least four ways of connection:

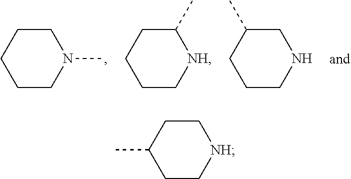

even if a H atom is drawn on —N—,

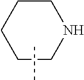

still includes the connection way of

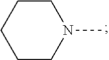

it's just that when one chemical bond is connected, the H at this site will be reduced by one, and the group will become the corresponding monovalent piperidinyl group;

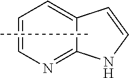

indicates that any linkable site on the pyridopyrazole group can be connected to other groups through one chemical bond, including at least six ways of connection:

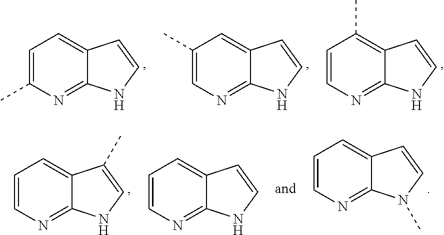

Unless otherwise specified, the number of atoms on a ring is usually defined as the number of members of the ring; for example, a “5- to 7-membered ring” refers to a “ring” with 5-7 atoms arranged in a circle.

Unless otherwise specified, the term “C₁₋₃ alkyl” is used to represent a linear or branched saturated hydrocarbon group composed of 1 to 3 carbon atoms. The C₁₋₃ alkyl includes C₁₋₂ alkyl, C₂₋₃ alkyl, etc. It may be monovalent (such as methyl), divalent (such as methylene) or multivalent (such as methenyl). Examples of the C₁₋₃ alkyl include, but are not limited to, methyl (Me), ethyl (Et), propyl (including n-propyl and isopropyl), etc.

Unless otherwise specified, the term “C₁₋₃ alkoxy” means alkyl groups containing 1 to 3 carbon atoms and attached to the remainder of a molecule by an oxygen atom. The C₁₋₃ alkoxy group includes C₁₋₂, C₂₋₃, C₃, and C₂ alkoxy groups, etc. Examples of C₁₋₃ alkoxy groups include, but are not limited to, methoxy, ethoxy, propoxy (including n-propoxy and isopropoxy), etc.

Unless otherwise specified, the terms “5- to 10-membered heteroaromatic ring” and “5- to 10-membered heteroaryl” may be used interchangeably. The term “5- to 10-membered heteroaryl” means a cyclic group having a conjugated pi electron system and composed of 5 to 10 ring atoms, in which 1, 2, 3 or 4 ring atoms are heteroatoms independently selected from O, S and N, and the remainder is carbon atoms. The 5- to 10-membered heteroaryl may be a monocyclic, fused bicyclic or fused tricyclic ring system, wherein each ring is aromatic, and wherein the nitrogen atom is optionally quaternized and the nitrogen and sulfur heteroatoms are optionally oxidized (i.e., NO and S(O)ₚ, wherein p is 1 or 2). A 5- to 10-membered heteroaryl can be attached to the remainder of the molecule through a heteroatom or a carbon atom. The 5- to 10-membered heteroaryl group includes 5- to 8-membered, 5- to 7-membered, 5- to 6-membered, 5-membered and 6-membered heteroaryl groups. Examples of the 5-10 membered heteroaryl include, but are not limited to, pyrrolyl (including N-pyrrolyl, 2-pyrrolyl, 3-pyrrolyl, etc.), pyrazolyl (including 2-pyrazolyl and 3-pyrazolyl, etc.), imidazolyl (including N-imidazolyl, 2-imidazolyl, 4-imidazolyl, and 5-imidazolyl, etc.), oxazolyl (including 2-oxazolyl, 4-oxazolyl, and 5-oxazolyl, etc.), triazolyl (1H-1,2,3-triazolyl, 2H-1,2,3-triazolyl, 1H-1,2,4-triazolyl and 4H-1,2,4-triazolyl, etc.), tetrazolyl, isoxazolyl (3-isoxazolyl, 4-isoxazolyl and 5-isoxazolyl, etc.), thiazolyl (including 2-thiazolyl, 4-thiazolyl and 5-thiazolyl, etc.), furyl (including 2-furyl and 3-furyl, etc.), thienyl (including 2-thienyl and 3-thienyl, etc.), pyridyl (including 2-pyridyl, 3-pyridyl and 4-pyridyl, etc.), pyrazinyl or pyrimidinyl (including 2-pyrimidinyl and 4-pyrimidinyl, etc.), benzothiazolyl (including 5-benzothiazolyl, etc.), purinyl, benzimidazolyl (including 2-benzimidazolyl, etc.), benzoxazolyl, indolyl (including 5-indolyl, etc.), isoquinolyl (including 1-isoquinolyl, 5-isoquinolyl, etc.), quinoxalinyl (including 2-quinoxalinyl, 5-quinoxalinyl, etc.) or quinolyl (including 3-quinolyl, 6-quinolyl, etc.).

Unless otherwise specified, “C₃₋₆ cycloalkyl” refers to a saturated cyclic hydrocarbon group consisting of 3 to 6 carbon atoms, which includes monocyclic and bicyclic systems. The C₃₋₆ cycloalkyl includes C₃₋₅, C₄₋₅, and C₅₋₆ cycloalkyl, etc., and may be monovalent, divalent, or polyvalent. Examples of C₃₋₆ cycloalkyl include, but are not limited to, cyclopropyl, cyclobutyl, cyclopentyl, cyclohexyl, etc.

Unless otherwise specified, the term “5- to 6-membered heterocycloalkenyl” alone or in combination with other terms each means a partially unsaturated cyclic group containing at least one carbon-carbon double bond and consisting of 5 to 6 ring atoms, of which 1, 2, 3 or 4 ring atoms are heteroatoms independently selected from O, S and N, and the remainder atoms are carbon atoms, wherein the nitrogen atom is optionally quaternized and the carbon, nitrogen and sulfur heteroatoms are optionally oxidized (i.e., C(═O), NO and S(O)ₚ, wherein p is 1 or 2).

The 5- to 6-membered heterocycloalkenyl includes monocyclic and bicyclic systems, wherein the bicyclic system includes spiro-, fused- and bridged-rings, and any ring of the system is non-aromatic. In addition, with respect to the “5- to 6-membered heterocycloalkenyl”, the heteroatom may be present at the position of attachment of the heterocycloalkenyl group to the remainder of a molecule. The 5- to 6-membered heterocycloalkenyl group includes 5-membered and 6-membered heterocycloalkenyl groups, etc. Examples of 5- to 6-membered heterocycloalkenyl groups include, but are not limited to,

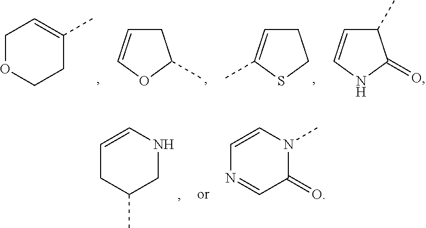

Unless otherwise specified, the terms “5-membered heteroaryl” and “5-membered heteroaromatic ring” may be used interchangeably in the present disclosure, and the term “5-membered heteroaryl” refers to a monocyclic group with a conjugated pi-electronic system consisting of five ring atoms, of which 1, 2, 3 or 4 ring atoms are heteroatoms independently selected from O, S, and N, and the rest are carbon atoms, wherein the nitrogen atom is optionally quaternized, and the carbon, nitrogen and sulfur heteroatoms may be optionally oxidized (i.e., C(═O), NO and S(O)ₚ, wherein p is 1 or 2). The 5-membered heteroaryl can be connected to the rest of the molecule by a heteroatom or carbon atom. Examples of the 5-membered heteroaryl include, but are not limited to, pyrrolyl (including N-pyrrolyl, 2-pyrrolyl, and 3-pyrrolyl, etc.), pyrazolyl (including 2-pyrazolyl and 3-pyrazolyl, etc.), imidazolyl (including N-imidazolyl, 2-imidazolyl, 4-imidazolyl and 5-imidazolyl, etc.), oxazolyl (including 2-oxazolyl, 4-oxazolyl and 5-oxazolyl, etc.), triazolyl (1H-1,2,3-triazolyl, 2H-1,2,3-triazolyl, 1H-1,2,4-triazolyl and 4H-1,2,4-triazolyl, etc.), tetrazolyl, isoxazolyl (3-isoxazolyl, 4-isoxazolyl and 5-isoxazolyl, etc.), thiazolyl (including 2-thiazolyl, 4-thiazolyl and 5-thiazolyl, etc.), furanyl (including 2-furanyl and 3-furanyl, etc.), thienyl (including 2-thienyl and 3-thienyl, etc.). Unless otherwise specified, “C₃₋₈ cycloalkyl” refers to a saturated cyclic hydrocarbon group consisting of 3 to 8 carbon atoms. The C₃₋₈ cycloalkyl includes monocyclic and bicyclic systems, wherein the bicyclic system includes spiro-, fused- and bridged-rings. The C₃₋₁₀ cycloalkyl includes C₃₋₆, C₃₋₅, C₄₋₈, C₄₋₆, C₄₋₅, C₅₋₈, C₅₋₆ and C₈₋₁₀ cycloalkyl, etc., and may be monovalent, divalent, or polyvalent. Examples of C₃₋₁₀ cycloalkyl include, but are not limited to, cyclopropyl, cyclobutyl, cyclopentyl, cyclohexyl, cycloheptyl, norbornyl, and [2.2.2]bicyclooctane, etc.

Unless otherwise specified, the term “5- to 10-membered heterocycloalkyl” alone or in combination with other terms each means a saturated cyclic group consisting of 5 to 10 ring atoms, of which 1, 2, 3 or 4 ring atoms are heteroatoms independently selected from O, S and N, and the remainder atoms are carbon atoms, wherein the nitrogen atom is optionally quaternized and the nitrogen and sulfur heteroatoms are optionally oxidized (i.e., NO and S(O)ₚ, wherein p is 1 or 2). The 5- to 10-membered heterocycloalkyl includes monocyclic and bicyclic systems, wherein the bicyclic system includes spiro-, fused- and bridged-rings. In addition, with respect to the “5- to 10-membered heterocycloalkyl”, the heteroatom may be present at the position of attachment of the heterocycloalkyl group to the remainder of a molecule. For example, the 5- to 10-membered heterocycloalkyl group includes, but is not limited to, 5- to 6-membered, 7-, 5-membered and 4-membered fused- or spiro-heterocycloalkyl, 5-membered and 4-membered bridged heterocycloalkyl, etc. Examples of 5- to 10-membered heterocycloalkyl include, but are not limited to, azetidinyl, oxetanyl, thietanyl, pyrrolidinyl, pyrazolidinyl, imidazolidinyl, tetrahydrothienyl (including tetrahydrothien-2-yl and tetrahydrothien-3-yl, etc.), tetrahydrofuranyl (including tetrahydrofuran-2-yl, etc.), tetrahydropyranyl, piperidinyl (including 1-piperidinyl, 2-piperidinyl and 3-piperidinyl, etc.), piperazinyl (including 1-piperazinyl and 2-piperazinyl, etc.), morpholinyl (including 3-morpholinyl and 4-morpholinyl, etc.), dioxanyl, dithianyl, isoxazolidinyl, isothiazolidinyl, 1,2-oxazinyl, 1,2-thiazinyl, hexahydropyridazinyl, homopiperazinyl, homopiperidinyl or dioxacycloheptyl, etc.

Unless otherwise specified, Cₙ₋ₙ₊ₘ or Cₙ—Cₙ₊ₘ includes any specific case of n to n+m carbons, for example, C₁₋₁₂ includes C₁, C₂, C₃, C₄, C₅, C₆, C₇, C₈, C₉, C₁₀, C₁1 and C₁₂, also includes any range from n to n+m, for example, C₁₋₁₂ includes C₁₋₃, C₁₋₆, C₁₋₉, C₃₋₆, C₃₋₉, C₃₋₁₂, C₆₋₉, C₆₋₁₂ and C₉₋₁₂, etc.; similarly, n membered to n+m membered indicates that the number of atoms on a ring is n to n+m, for example, 3-12 membered ring includes 3 membered ring, 4 membered ring, 5 membered ring, 6 membered ring, 7 membered ring, 8 membered ring, 9 membered ring, 10 membered ring, 11 membered ring, and 12 membered ring, also includes any range from n to n+m, for example, 3-12 membered ring includes 3-6 membered ring, 3-9 membered ring, 5-6 membered ring, 5-7 membered ring, 6-7 membered ring, 6-8 membered ring, and 6-10 membered ring, etc.

The term “leaving group” refers to a functional group or atom which can be replaced by another functional group or atom through a substitution reaction (such as nucleophilic substitution reaction). For example, representative leaving groups include triflate; chlorine, bromine and iodine; sulfonate group, such as mesylate, tosylate, p-bromobenzenesulfonate, p-toluenesulfonate and the like; acyloxy, such as acetoxy, trifluoroacetoxy and the like.

The term “protecting group” includes, but is not limited to, “amino protecting group”, “hydroxy protecting group” or “thio protecting group”. The term “amino protecting group” refers to a protecting group suitable for blocking the side reaction on the nitrogen of an amino.

Representative amino protecting groups include, but are not limited to: formyl; acyl, such as alkanoyl (e.g., acetyl, trichloroacetyl or trifluoroacetyl); alkoxycarbonyl, such as tert-butoxycarbonyl (Boc); arylmethoxycarbonyl such as benzyloxycarbonyl (Cbz) and 9-fluorenylmethoxycarbonyl (Fmoc); arylmethyl such as benzyl (Bn), trityl (Tr), 1,1-bis-(4′-methoxyphenyl)methyl; silyl such as trimethylsilyl (TMS) and tert-butyldimethylsilyl (TBS), etc. The term “hydroxy protecting group” refers to a protecting group suitable for blocking the side reaction on hydroxy. Representative hydroxy protecting groups include, but are not limited to: alkyl such as methyl, ethyl and tert-butyl; acyl such as alkanoyl (e.g., acetyl); arylmethyl such as benzyl (Bn), p-methoxybenzyl (PMB), 9-fluorenylmethyl (Fm), and diphenylmethyl (benzhydryl, DPM); silyl such as trimethylsilyl (TMS) and tert-butyl dimethyl silyl (TBS), etc.

Compounds disclosed herein can be prepared by a variety of synthetic methods well known to those skilled in the art, including the following enumerated embodiment, the embodiment formed by the following enumerated embodiment in combination with other chemical synthesis methods, and equivalent replacement well known to those skilled in the art. Alternative embodiments include, but are not limited to the embodiment disclosed herein.

The structures of compounds disclosed herein can be confirmed by conventional methods well known to those skilled in the art. If the present disclosure relates to an absolute configuration of a compound, the absolute configuration can be confirmed by conventional techniques in the art, such as single crystal X-Ray diffraction (SXRD). In the single crystal X-Ray diffraction (SXRD), the diffraction intensity data of the cultivated single crystal is collected using a Bruker D8 venture diffractometer with a light source of CuKα radiation in a scanning mode of φ/ω scan; after collecting the relevant data, the crystal structure is further analyzed by the direct method (Shelxs97) to confirm the absolute configuration.

Solvents used in the present disclosure are commercially available. The following abbreviations are used in the present disclosure: aq represents aqueous; CDCl₃ represents deuterated chloroform; KF represents potassium fluoride; and psi represents pounds per square inch, a unit of pressure.

### DETAILED DESCRIPTION OF THE INVENTION

The present disclosure is described in detail below by means of examples. However, it is not intended that these examples have any disadvantageous limitations to the present disclosure. The present disclosure has been described in detail herein, and embodiments are also disclosed herein. It will be apparent to those skilled in the art that various changes and modifications may be made to the embodiments disclosed herein without departing from the spirit and scope disclosed herein.

### Intermediate 1

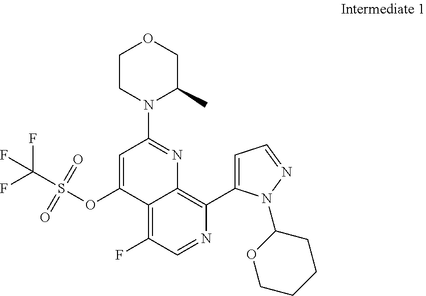 [SMILES: C[C@@H]1COCCN1c1cc(OS(=O)(=O)C(F)(F)F)c2c(F)cnc(-c3ccnn3C3CCCCO3)c2n1]

Synthetic route:

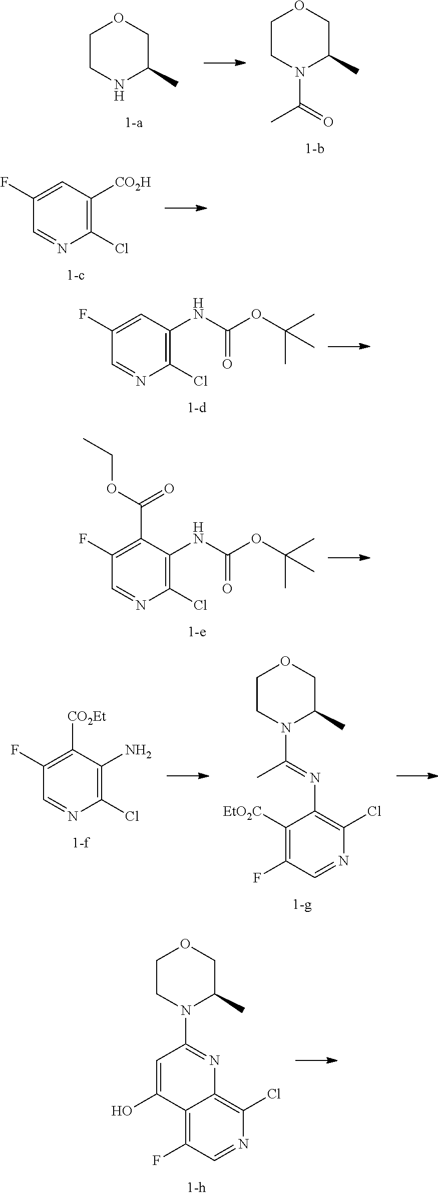

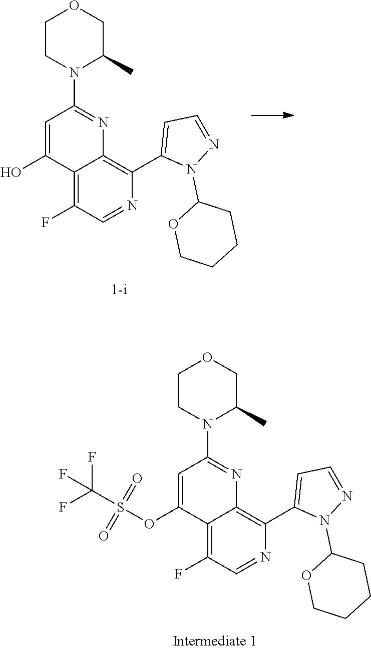 [SMILES: C[C@@H]1COCCN1c1cc(O)c2c(F)cnc(-c3ccnn3C3CCCCO3)c2n1 ; C[C@@H]1COCCN1c1cc(OS(=O)(=O)C(F)(F)F)c2c(F)cnc(-c3ccnn3C3CCCCO3)c2n1]

Step 1: Synthesis of Compound 1-b

Compound 1-a (15 g, 148.30 mmol) was dissolved in dichloromethane (100 mL), and triethylamine (18.01 g, 177.96 mmol, 24.77 mL) was added. The air in the reaction system was replaced with nitrogen, and the mixture was cooled down to 0-5° C. Acetyl chloride (12.81 g, 163.13 mmol, 11.64 mL) was added dropwise at controlled temperature. After the dropwise addition was completed, the mixture was returned to room temperature and stirred for 0.5 hours. The reaction mixture was quenched with water (70 mL) at 0-5° C., and extracted with dichloromethane (50 mL×3). The organic phase was dried over anhydrous sodium sulfate, and filtered. The filtrate was concentrated under reduced pressure to afford a crude product. The crude product was purified by column chromatography (eluent: ethyl acetate) to afford compound 1-b.

Step 2: Synthesis of Compound 1-d

Compound 1-c (5 g, 28.48 mmol) was dissolved in toluene (50 mL), and triethylamine (5.76 g, 56.97 mmol, 7.93 mL), tert-butanol (6.33 g, 85.45 mmol, 8.17 mL) and diphenyl azidophosphate (9.41 g, 34.18 mmol, 7.41 mL) were added sequentially. The mixture was heated to 110° C. and reacted for 0.5 h. The mixture was cooled down to room temperature, and 200 mL of water was added. The mixture was extracted with ethyl acetate (150 mL), and the layers were separated. The organic phase was dried over anhydrous sodium sulfate, and filtered.

The filtrate was rotary-evaporated to dryness. The crude product was purified by column chromatography (eluent: ethyl acetate:petroleum ether=1:8) to afford compound 1-d.

¹H NMR (400 MHz, CDCl₃) δ 8.40 (dd, J=2.8 Hz, J=10.0 Hz, 1H), 7.92 (d, J=2.8 Hz, 1H), 7.05 (s, 1H), 1.55 (s, 9H)

Step 3: Synthesis of Compound 1-e

Compound 1-d (5 g, 20.27 mmol) was dissolved in tetrahydrofuran (50 mL), and the solution was cooled down to −70° C. under nitrogen. Lithium diisopropylamide (2 M, 23.31 mL) was added dropwise, and the mixture was stirred at −70° C. for 1 h. Ethyl chloroformate (2.99 g, 27.55 mmol, 2.62 mL) was added dropwise, and the mixture was reacted at −70° C. for 1 h.

Saturated aqueous sodium carbonate (70 mL) was slowly added dropwise to the reaction solution, and the mixture was extracted with ethyl acetate (50 mL×2). The organic phase was washed with saturated saline (100 mL), dried over anhydrous sodium sulfate, and filtered. The filtrate was rotary-evaporated to dryness. The crude product was purified by column chromatography (eluent: petroleum ether:ethyl acetate=20:1) to afford compound 1-e.

Step 4: Synthesis of Compound 1-f

Compound 1-e (9.2 g, 28.86 mmol) was dissolved in dichloromethane (10 mL) and hydrochloric acid/ethyl acetate (4 M, 100.00 mL) was added dropwise. The mixture was stirred at room temperature overnight. The reaction solution was concentrated under reduced pressure to remove the solvent, and dichloromethane (100 mL) was added. The mixture was adjusted to a pH of 8 with saturated aqueous sodium bicarbonate, and the aqueous phase was further extracted with dichloromethane (50 mL). The organic phase was washed with saturated saline (100 mL), dried over anhydrous sodium sulfate, and filtered. The filtrate was rotary-evaporated to dryness. The crude product was purified by column chromatography (eluent: petroleum ether:ethyl acetate=20:1) to afford compound 1-f

Step 5: Synthesis of Compound 1-g

Compound 1-b (3.96 g, 27.67 mmol) was dissolved in 1,2-dichloroethane (100 mL), and the mixture was cooled down to 0° C. under nitrogen. Phosphorus oxychloride (11.57 g, 75.48 mmol, 7.01 mL) was added dropwise, and the mixture was stirred at room temperature for 3 h. Compound 1-f (5.5 g, 25.16 mmol) was added, and the mixture was heated to 80° C. and stirred overnight. The reaction solution was cooled down to room temperature, and saturated aqueous sodium carbonate solution (300 mL) was slowly added dropwise. The mixture was extracted with dichloromethane (100 mL×3). The organic phase was washed with saturated saline (100 mL), dried over anhydrous sodium sulfate, and filtered. The filtrate was rotary-evaporated to dryness. The crude product was purified by column chromatography (eluent: petroleum ether:ethyl acetate=20:1˜1:1) to afford compound 1-g.

Step 6: Synthesis of Compound 1-h

Compound 1-g (6.8 g, 19.78 mmol) was dissolved in tetrahydrofuran (100 mL), and the air in the reaction system was replaced 3 times with nitrogen. The mixture was cooled to 0° C., and lithium hexamethyldisilazane (1 M, 59.34 mL) was added dropwise at 0 to 10° C.

The mixture was slowly warmed to room temperature and stirred for 1 h, and then heated to 70° C. and stirred overnight. The reaction solution was cooled down to room temperature, and water (100 mL) was added. The mixture was adjusted to a pH of 6˜7 with 1N dilute hydrochloric acid, and extracted with dichloromethane (100 mL×2). The organic phase was washed with saturated saline (100 mL), dried over anhydrous sodium sulfate, and filtered. The filtrate was rotary-evaporated to dryness. The crude product was purified by column chromatography (eluent: petroleum ether:ethyl acetate=1:1) to afford compound 1-h.

Step 7: Synthesis of Compound 1-i

Compound 1-h (3 g, 10.08 mmol), 1-(2-tetrahydropyranyl)-1H-pyrazole-5-boronic acid pinacol ester (5.61 g, 20.15 mmol) and potassium carbonate (5.57 g, 40.31 mmol) were dissolved in a solvent mixture of 1,4-dioxane (50 mL) and water (5 mL), and the air in the reaction system was replaced 3 times with nitrogen. [1,1′-bis(diphenylphosphino)ferrocene]palladium dichloride (737.33 mg, 1.01 mmol) was added under nitrogen and the mixture was heated to 100° C. and stirred for 30 h. The reaction solution was cooled down to room temperature, and concentrated under reduced pressure to remove most of the organic solvent. Water (100 mL) was added, and the mixture was extracted with dichloromethane (100 mL×3). The organic phase was washed with saturated saline (100 mL), dried over anhydrous sodium sulfate, and filtered. The filtrate was rotary-evaporated to dryness. The crude product was purified by column chromatography (eluent: petroleum ether:ethyl acetate=1:1˜1:2) to afford compound 1-i.

Step 8: Synthesis of Intermediate 1

Compound 1-i (0.2 g, 483.74 μmol) and N,N-diisopropylethylamine (156.30 mg, 1.21 mmol, 210.65 μL) were dissolved in dichloromethane (5 mL), and the mixture was cooled down to 0° C. under nitrogen. A solution of N-Phenyl-bis(trifluoromethanesulfonimide) (259.22 mg, 725.61 μmol) in dichloromethane (2 mL) was added dropwise, and the mixture was stirred for 1 h while maintaining the temperature. The mixture was slowly warmed to room temperature and stirred overnight. Saturated aqueous ammonium chloride solution (10 mL) was added and the mixture was extracted with dichloromethane (5 mL). The organic phase was dried over anhydrous sodium sulfate, and filtered. The filtrate was rotary-evaporated to dryness.

The crude product was purified by preparative thin layer chromatography (eluent: petroleum ether:ethyl acetate=1:2) to afford intermediate 1.

¹H NMR (400 MHz, CDCl₃) δ 8.35 (t, J=2.0 Hz, 1H), 7.69 (d, J=1.2 Hz, 1H), 7.03 (d, J=3.6 Hz, 1H), 6.89˜6.83 (m, 1H), 5.95˜5.92 (m, 1H), 4.43˜4.36 (m, 1H), 4.27˜4.23 (m, 1H), 4.09˜4.03 (m, 1H), 3.99˜3.93 (m, 1H), 3.86˜3.81 (m, 1H), 3.76˜3.69 (m, 1H), 3.62˜3.53 (m, 1H), 3.49˜3.28 (m, 2H), 2.57˜2.49 (m, 1H), 2.10˜2.08 (m, 2H), 1.76˜1.67 (m, 2H), 1.53˜1.50 (m, 1H), 1.35 (d, J=6.8 Hz, 3H)

### Example 1

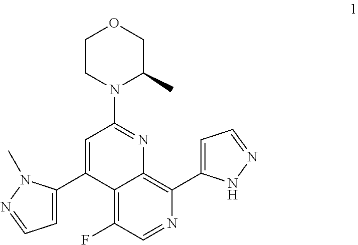 [SMILES: C[C@@H]1COCCN1c1cc(-c2ccnn2C)c2c(F)cnc(-c3ccn[nH]3)c2n1]

Synthetic route:

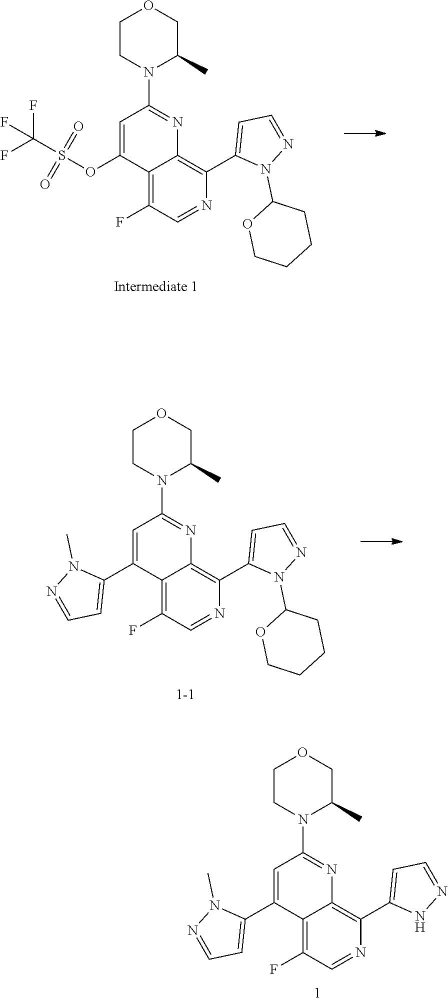 [SMILES: C[C@@H]1COCCN1c1cc(OS(=O)(=O)C(F)(F)F)c2c(F)cnc(-c3ccnn3C3CCCCO3)c2n1 ; C[C@@H]1COCCN1c1cc(-c2ccnn2C)c2c(F)cnc(-c3ccnn3C3CCCCO3)c2n1 ; C[C@@H]1COCCN1c1cc(-c2ccnn2C)c2c(F)cnc(-c3ccn[nH]3)c2n1]

Step 1: Synthesis of Compound 1-1

Intermediate 1 (0.1 g, 183.32 μmol), 1-methyl-1H-pyrazole-5-boronic acid pinacol ester (57.21 mg, 274.97 μmol), 4A molecular sieve (0.1 g) and cesium carbonate (119.46 mg, 366.63 μmol) were dissolved in toluene (2 mL), and the air in the reaction system was replaced 3 times with nitrogen. [2′-(amino)[1,1′-biphenyl]-2-yl][dicyclohexyl[3,6-dimethoxy-2′,4′,6′-tris(1-methylethyl) [1,1′-biphenyl]-2-yl]phosphine](methanesulfonato)palladium (16.62 mg, 18.33 μmol) was added under nitrogen, and the mixture was heated to 110° C. and stirred overnight. The reaction solution was cooled down to room temperature, and ethyl acetate (10 mL) was added. The mixture was filtered. The filter cake was rinsed with ethyl acetate (5 mL×2). The filtrate was washed respectively with 1N aqueous sodium hydroxide (5 mL×2) and saturated saline (5 mL), dried over anhydrous sodium sulfate, and filtered. The filtrate was rotary-evaporated to dryness. The crude product was purified by preparative thin layer chromatography (eluent: petroleum ether:ethyl acetate=1:2) to afford compound 1-1.

Step 2: Synthesis of Compound 1

Compound 1-1 (27 mg, 56.54 μmol) was dissolved in trifluoroacetic acid (1 mL) and the mixture was stirred at room temperature for 0.5 h. The mixture was concentrated under reduced pressure to remove the solvent. The crude product was purified by reversed-phase column (trifluoroacetic acid), and the fraction was concentrated under reduced pressure to remove most of the acetonitrile. The aqueous phase was adjusted to a pH of 7˜8 with saturated aqueous sodium bicarbonate solution. The mixture was extracted with dichloromethane (10 mL×3). The organic phase was washed with saturated saline (10 mL), dried over anhydrous sodium sulfate, and filtered. The filtrate was rotary-evaporated to dryness to afford compound 1.

¹H NMR (400 MHz, CDCl₃) δ 8.24 (d, J=2.0 Hz, 1H), 7.72 (d, J=1.6 Hz, 1H), 7.60 (d, J=2.0 Hz, 1H), 7.28 (d, J=1.6 Hz, 1H), 7.14 (s, 1H), 6.38 (d, J=2.0 Hz, 1H), 4.50˜4.40 (m, 1H), 4.20˜4.16 (m, 1H), 4.07˜4.04 (m, 1H), 3.95˜3.83 (m, 2H), 3.74˜3.69 (m, 4H), 3.61˜3.54 (m, 1H), 1.48 (d, J=6.8 Hz, 3H)

### Example 2

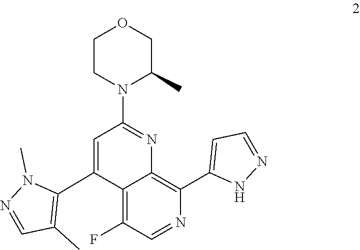 [SMILES: Cc1cnn(C)c1-c1cc(N2CCOC[C@H]2C)nc2c(-c3ccn[nH]3)ncc(F)c12]

Synthetic route:

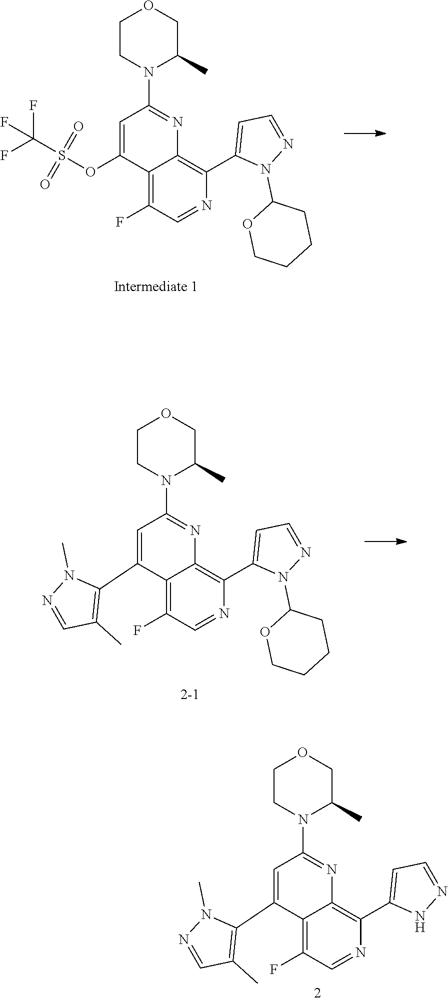 [SMILES: C[C@@H]1COCCN1c1cc(OS(=O)(=O)C(F)(F)F)c2c(F)cnc(-c3ccnn3C3CCCCO3)c2n1 ; Cc1cnn(C)c1-c1cc(N2CCOC[C@H]2C)nc2c(-c3ccnn3C3CCCCO3)ncc(F)c12 ; Cc1cnn(C)c1-c1cc(N2CCOC[C@H]2C)nc2c(-c3ccn[nH]3)ncc(F)c12]

Step 1: Synthesis of Compound 2-1

Intermediate 1 (0.1 g, 183.32 μmol), 1,4-dimethylpyrazole-5-boronic acid pinacol ester (61.07 mg, 274.97 μmol), 4A molecular sieve (0.1 g) and cesium carbonate (119.46 mg, 366.63 μmol) were dissolved in toluene (2 mL), and the air in the reaction system was replaced 3 times with nitrogen. BrettPhos-Pd-G3 (16.62 mg, 18.33 μmol) was added under nitrogen and the mixture was heated to 110° C. and stirred overnight. The reaction solution was cooled down to room temperature, and ethyl acetate (10 mL) was added. The mixture was filtered. The filter cake was rinsed with ethyl acetate (5 mL×2). The filtrate was washed respectively with 1N aqueous sodium hydroxide (5 mL×2) and saturated saline (5 mL), dried over anhydrous sodium sulfate, and filtered. The filtrate was rotary-evaporated to dryness. The crude product was purified by preparative thin layer chromatography (eluent: petroleum ether:ethyl acetate=1:2) to afford compound 2-1.

Step 2: Synthesis of Compound 2

Compound 2-1 (50 mg, 101.72 μmol) and anisole (109.99 mg, 1.02 mmol, 110.55 μL) were dissolved in trifluoroacetic acid (770.00 mg, 6.75 mmol, 500.00 μL) and the mixture was stirred at room temperature for 1 h. The reaction solution was concentrated under reduced pressure to remove the solvent. The crude product was purified by reversed-phase column (trifluoroacetic acid), and the fraction was concentrated under reduced pressure to remove most of the acetonitrile. The aqueous phase was adjusted to a pH of 7-8 with saturated aqueous sodium bicarbonate solution. The mixture was extracted with dichloromethane (10 mL×3). The organic phase was washed with saturated saline (10 mL×3), dried over anhydrous sodium sulfate, and filtered. The filtrate was rotary-evaporated to dryness to afford compound 2.

¹H NMR (400 MHz, CDCl₃) δ 8.24 (s, 1H), 7.73 (s, 1H), 7.45 (s, 1H), 7.29 (s, 1H), 7.08 (d, J=3.2 Hz, 1H), 4.48˜4.37 (m, 1H), 4.21˜4.18 (m, 1H), 4.10˜4.07 (m, 1H), 3.96˜3.86 (m, 2H), 3.76˜3.70 (m, 1H), 3.65˜3.55 (m, 4H), 1.97 (s, 3H), 1.48 (d, J=6.8 Hz, 3H)

### Example 3

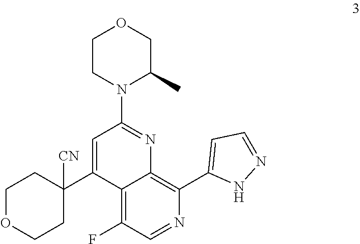 [SMILES: C[C@@H]1COCCN1c1cc(C2(C#N)CCOCC2)c2c(F)cnc(-c3ccn[nH]3)c2n1]

Synthetic route:

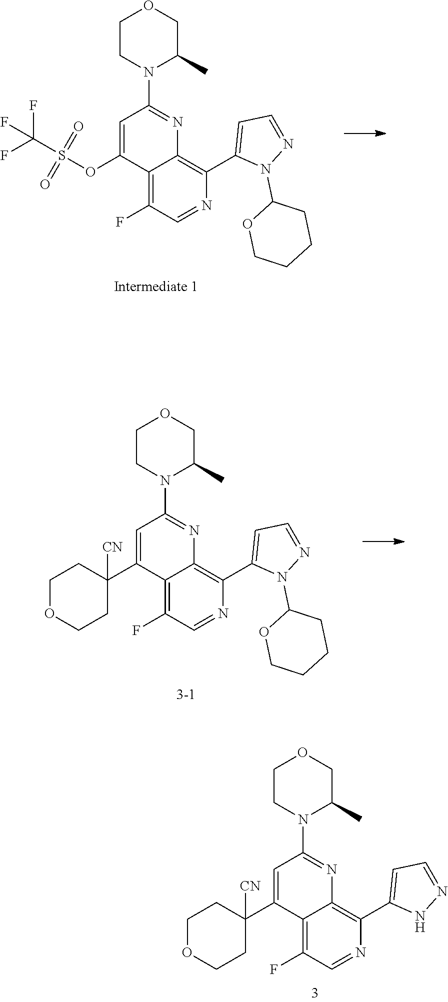 [SMILES: C[C@@H]1COCCN1c1cc(OS(=O)(=O)C(F)(F)F)c2c(F)cnc(-c3ccnn3C3CCCCO3)c2n1 ; C[C@@H]1COCCN1c1cc(C2(C#N)CCOCC2)c2c(F)cnc(-c3ccnn3C3CCCCO3)c2n1 ; C[C@@H]1COCCN1c1cc(C2(C#N)CCOCC2)c2c(F)cnc(-c3ccn[nH]3)c2n1]

Step 1: Synthesis of Compound 3-1

4-cyanotetrahydropyran (122.24 mg, 1.10 mmol) was dissolved in tetrahydrofuran (2 mL), and the mixture was cooled down to −60° C. under nitrogen. Lithium diisopropylamide (2 M, 604.94 μL) was added dropwise, and the mixture was stirred for 1 h while maintaining the temperature. A solution of intermediate 1 (0.3 g, 549.95 μmol) in tetrahydrofuran (2 mL) was added dropwise, and the mixture was stirred for 1 h while maintaining the temperature. Saturated aqueous ammonium chloride (5 mL) was added slowly dropwise to the reaction solution and the mixture was extracted with ethyl acetate (5 mL×3). The organic phase was washed with 1N aqueous sodium hydroxide (10 mL×2) to remove by-products of hydrolysis, then washed with saturated saline (10 mL), dried over anhydrous sodium sulfate, and filtered. The filtrate was rotary-evaporated to dryness. The crude product was purified by preparative thin layer chromatography (eluent: petroleum ether:ethyl acetate=1:3) to afford compound 3-1.

Step 2: Synthesis of Compound 3

Compound 3-1 (43 mg, 84.88 μmol) was dissolved in dichloromethane (1 mL) and trifluoroacetic acid (1.54 g, 13.51 mmol, 1 mL) was added. The mixture was stirred at room temperature for 3 h. The mixture was concentrated under reduced pressure to remove the solvent. The crude product was purified by reversed-phase column (trifluoroacetic acid), and the fraction was concentrated under reduced pressure to remove most of the acetonitrile. The aqueous phase was adjusted to a pH of 7˜8 with saturated aqueous sodium bicarbonate solution. The mixture was extracted with dichloromethane (10 mL×2). The organic phase was washed with saturated saline (10 mL), dried over anhydrous sodium sulfate, and filtered. The filtrate was rotary-evaporated to dryness to afford compound 3.

¹H NMR (400 MHz, CDCl₃) δ 8.36 (d, J=3.6 Hz, 1H), 7.72 (s, 1H), 7.29˜7.27 (m, 2H), 4.50˜4.41 (m, 1H), 4.22˜4.16 (m, 3H), 4.11˜4.02 (m, 3H), 3.97˜3.94 (m, 1H), 3.88˜3.84 (m, 1H), 3.75˜3.68 (m, 1H), 3.62˜3.55 (m, 1H), 2.47˜2.44 (m, 2H), 2.22˜2.12 (m, 2H), 1.48 (d, J=6.8 Hz, 3H)

### Example 4

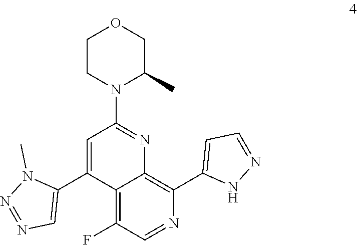 [SMILES: C[C@@H]1COCCN1c1cc(-c2cnnn2C)c2c(F)cnc(-c3ccn[nH]3)c2n1]

Synthetic route:

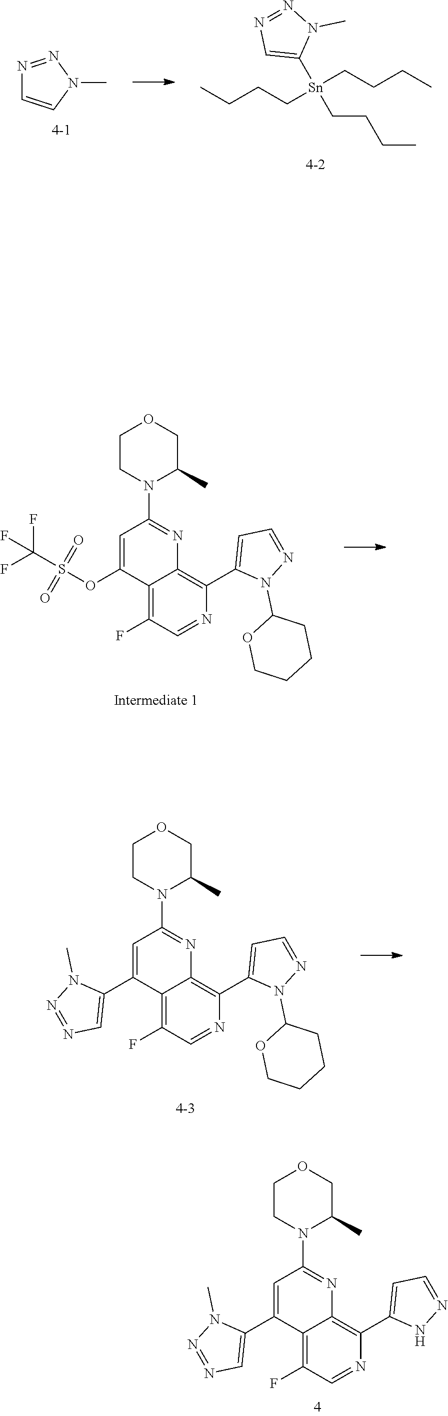

Step 1: Synthesis of Compound 4-2

Compound 4-1 (0.10 g, 1.20 mmol) was dissolved in tetrahydrofiran (1 mL), and the mixture was cooled to −78° C. under nitrogen. n-Butyllithium (2.5 M, 543.98 μL) was added dropwise, and the mixture was stirred for 1 h while maintaining the temperature. A solution of tri-n-butyltin chloride (550.0 mg, 1.69 mmol, 454.55 μL) in tetrahydrofuran (1 mL) was added dropwise at −78° C., and the mixture was stirred for 1 h while maintaining the temperature. The mixture was then slowly warmed to 25° C. and stirred for 2 hours. The reaction solution was cooled down to 0° C. and poured into aqueous ammonium chloride solution (10 mL). The mixture was extracted with ethyl acetate (10 mL×3). The organic phase was washed with saturated saline, dried over anhydrous sodium sulfate, and filtered. The filtrate was rotary-evaporated to dryness. The crude product was purified by column chromatography (eluent: petroleum ether:ethyl acetate=6:1) to afford compound 4-2.

Step 2: Synthesis of Compound 4-3

Intermediate 1 (140.0 mg, 256.64 μmol), compound 4-2 (191.01 mg, 513.28 μmol), cuprous iodide (7.33 mg, 38.50 μmol) and triethylamine (77.91 mg, 769.93 μmol, 107.16 μL) were dissolved in N,N-dimethylformamide (2 mL), and the air in the reaction system was replaced with nitrogen. Under nitrogen protection, tetrakis(triphenylphosphine)palladium (29.66 mg, 25.66 μmol) was added, and the mixture was heated to 100° C. and stirred overnight.

The mixture was cooled down to room temperature. The reaction mixture was quenched by adding 10% KF aqueous solution (10 mL), and 25% ammonia water (2 mL) was added. The mixture was extracted with ethyl acetate (10 mL×3). The organic phase was washed with saturated saline (10 mL×3), dried over anhydrous sodium sulfate, and filtered. The filtrate was rotary-evaporated to dryness. The crude product was purified by preparative thin layer chromatography (eluent: ethyl acetate) to afford compound 4-3.

Step 3: Synthesis of Compound 4

Compound 4-3 (60.0 mg, 125.39 μmol) was dissolved in dichloromethane (1 mL), and trifluoroacetic acid (1.54 g, 13.51 mmol, 1 mL) was added at room temperature. The mixture was stirred overnight. The reaction solution was concentrated to remove the solvent to afford a crude product. The crude product was purified by reversed-phase column (trifluoroacetic acid), and the fraction was concentrated under reduced pressure to remove most of the acetonitrile.

The aqueous phase was adjusted to a pH of 8 with saturated aqueous sodium bicarbonate solution. The mixture was extracted with dichloromethane (30 mL×2). The organic phase was washed with saturated saline (30 mL), dried over anhydrous sodium sulfate, and filtered. The filtrate was rotary-evaporated to dryness to afford compound 4.

¹H NMR (400 MHz, CDCl₃) δ 8.27 (s, 1H), 7.80 (s, 1H), 7.74 (s, 1H), 7.32 (s, 1H), 7.14 (s, 1H), 4.47˜4.46 (m, 1H), 4.20˜4.19 (m, 1H), 4.08˜4.05 (m, 1H), 3.96-3.84 (m, 5H), 3.74-3.69 (m, 1H), 3.61-3.56 (m, 1H), 1.49 (d, J=6.8 Hz, 3H)

### Example 5

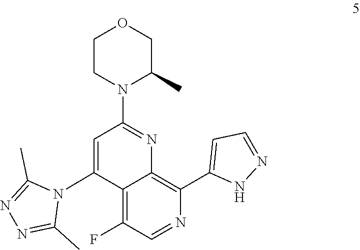 [SMILES: Cc1nnc(C)n1-c1cc(N2CCOC[C@H]2C)nc2c(-c3ccn[nH]3)ncc(F)c12]

Synthetic route:

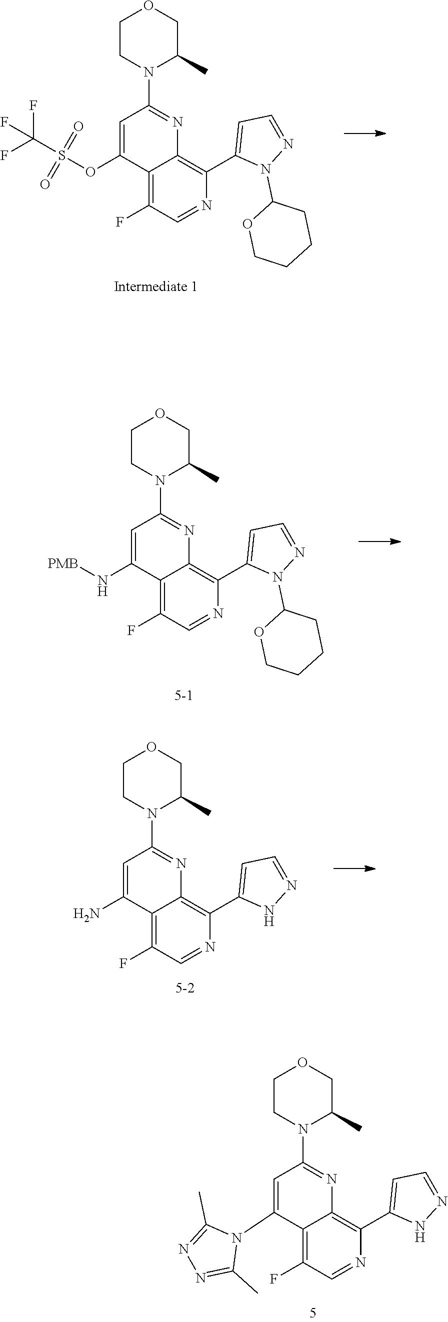

Step 1: Synthesis of Compound 5-1

Intermediate 1(1.02 g, 1.87 mmol) and p-methoxybenzylamine (2.57 g, 18.70 mmol, 2.42 mL) were dissolved in acetonitrile (10 mL), and the mixture was heated to 90° C. and stirred overnight. The mixture was concentrated under reduced pressure to afford a crude product. The crude product was purified by column chromatography (eluent: petroleum ether:ethyl acetate=1:1) to afford compound 5-1.

Step 2: Synthesis of Compound 5-2

Compound 5-1 (430 mg, 807.35 μmol) was dissolved in trifluoroacetic acid (4.62 g, 40.52 mmol, 3 mL), and the mixture was heated to 60° C. and stirred overnight. The reaction solution was concentrated under reduced pressure. The residue was dissolved in dichloromethane (50 mL), and the mixture was adjusted to a pH of 8-9 with saturated aqueous sodium bicarbonate solution. The mixture was extracted with dichloromethane (50 mL×3). The organic phase was washed with saturated saline (50 mL), dried over anhydrous sodium sulfate, and filtered. The filtrate was rotary-evaporated to dryness to afford the crude compound 5-2.

Step 3: Synthesis of Compound 5

1,1-Dimethoxy-N,N-dimethylethylamine (1.40 g, 10.51 mmol, 1.54 mL) and compound 5-2 (300 mg, 913.68 μmol) were dissolved in N,N-dimethylformamide (6 mL), and the mixture was heated to 50° C. and stirred overnight. The mixture was cooled down to room temperature, and water (20 mL) was added. The mixture was extracted with ethyl acetate (20 mL×3). The organic phase was washed with saturated brine (20 mL), dried over anhydrous sodium sulfate, and filtered. The filtrate was rotary-evaporated to dryness to afford a crude product. The crude product was dissolved in acetic acid (10 mL), and the mixture was heated to 75° C. and stirred overnight. The reaction solution was concentrated to remove the solvent, and dichloromethane (20 mL) was added. The mixture was adjusted to a pH of 7˜8 with saturated aqueous sodium bicarbonate solution, and extracted with dichloromethane (20 mL×3). The organic phase was washed with saturated saline, dried over anhydrous sodium sulfate, and filtered. The filtrate was rotary-evaporated to dryness. The crude product was purified by reversed-phase column (trifluoroacetic acid), and the fraction was concentrated under reduced pressure to remove most of the acetonitrile. The aqueous phase was adjusted to a pH of 8 with saturated aqueous sodium bicarbonate solution, and extracted with dichloromethane (30 mL×2). The organic phase was washed with saturated saline (30 mL), dried over anhydrous sodium sulfate, and filtered. The filtrate was rotary-evaporated to dryness. The crude product was stirred with methyl tert-butyl ether (5 mL) and filtered. The filter cake was washed with methyl tert-butyl ether (1 mL×3) and collected to afford compound 5.

^{H} NMR (400 MHz, MeOD) δ 8.28 (d, J=2.0 Hz, 1H), 7.75 (s, 1H), 7.72 (s, 1H), 7.41 (s, 1H), 4.66˜4.65 (m, 1H), 4.27˜4.23 (m, 1H), 4.13˜4.11 (m, 1H), 3.93˜3.83 (m, 2H), 3.74˜3.67 (m, 1H), 3.57˜3.50 (m, 1H), 2.30 (s, 6H), 1.45 (d, J=6.8 Hz, 3H)

### Example 6

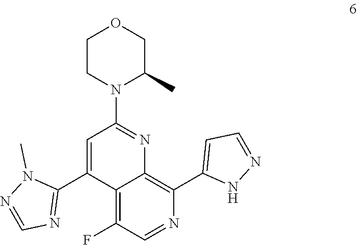 [SMILES: C[C@@H]1COCCN1c1cc(-c2ncnn2C)c2c(F)cnc(-c3ccn[nH]3)c2n1]

Synthetic route:

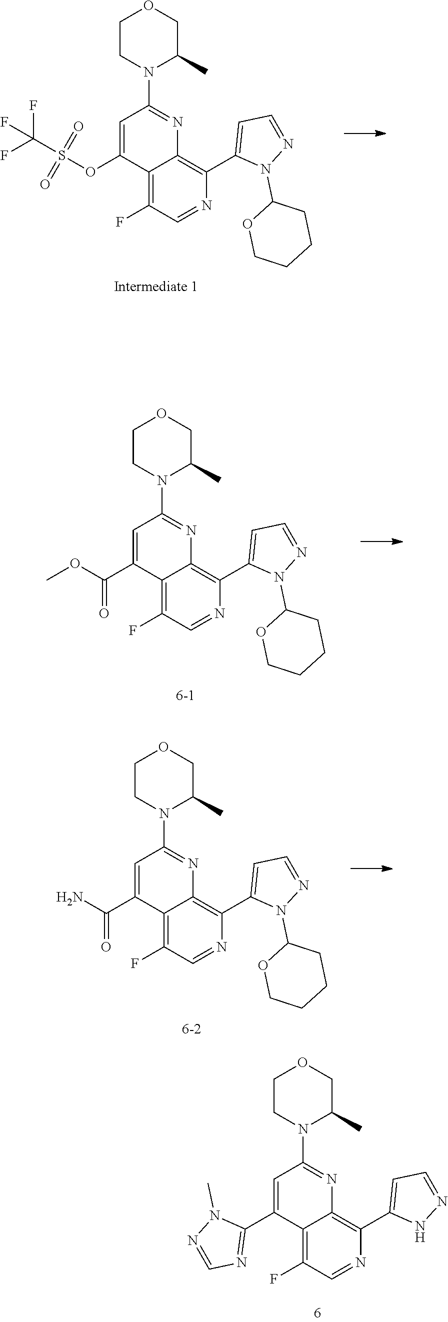 [SMILES: C[C@@H]1COCCN1c1cc(OS(=O)(=O)C(F)(F)F)c2c(F)cnc(-c3ccnn3C3CCCCO3)c2n1 ; COC(=O)c1cc(N2CCOC[C@H]2C)nc2c(-c3ccnn3C3CCCCO3)ncc(F)c12 ; C[C@@H]1COCCN1c1cc(C(N)=O)c2c(F)cnc(-c3ccnn3C3CCCCO3)c2n1 ; C[C@@H]1COCCN1c1cc(-c2ncnn2C)c2c(F)cnc(-c3ccn[nH]3)c2n1]

Step 1: Synthesis of Compound 6-1

Intermediate 1 (1 g, 1.83 mmol) was dissolved in a mixed solvent of N,N-dimethylformamide (13 mL) and methanol (7 mL), and triethylamine (370.99 mg, 3.67 mmol, 510.30 μL), palladium acetate (41.16 mg, 183.32 μmol) and 1,3-bis(diphenylphosphino)propane (75.61 mg, 183.32 μmol) were added. The air in the reaction system was replaced 3 times with carbon monoxide, and the mixture was heated to 80° C. and stirred at 50 psi overnight. The reaction solution was directly concentrated to remove the solvent. The crude product was purified by column chromatography (eluent: petroleum ether:ethyl acetate=1:1) to afford compound 6-1.

Step 2: Synthesis of Compound 6-2

Compound 6-1 (0.5 g, 1.10 mmol) was dissolved in ammonia methanol solution (7 M, 15 mL), and the mixture was heated to 80° C. and stirred overnight. The mixture was concentrated under reduced pressure to remove the solvent. The crude product was purified by preparative thin layer chromatography (eluent: ethyl acetate) to afford compound 6-2.

Step 3: Synthesis of Compound 6

Compound 6-2 (180 mg, 408.65 μmol) was dissolved in N,N-dimethylformamide (2 mL), and N,N-dimethylformamide dimethyl acetal (486.96 mg, 4.09 mmol, 542.88 μL) was added. The mixture was heated to 95° C. and stirred for 0.5 hours. The reaction solution was concentrated to remove the solvent to afford a crude product. The crude product and methylhydrazine (1.24 g, 10.77 mmol, 1.42 mL, 40% purity) were dissolved in acetic acid (10 mL), and the mixture was heated to 90° C. and stirred overnight. The mixture was cooled down to room temperature, and concentrated to remove the solvent. Dichloromethane (20 mL) was added, and the mixture was adjusted to a pH of 7˜8 with saturated aqueous sodium bicarbonate solution. The mixture was extracted with dichloromethane (20 mL×3). The organic phase was washed with saturated saline, dried over anhydrous sodium sulfate, and filtered. The filtrate was rotary-evaporated to dryness. The crude product was purified by reversed-phase column (trifluoroacetic acid), and the fraction was concentrated under reduced pressure to remove most of the acetonitrile. The aqueous phase was adjusted to a pH of 8 with saturated aqueous sodium bicarbonate solution, and extracted with dichloromethane (30 mL×2). The organic phase was washed with saturated saline (30 mL), dried over anhydrous sodium sulfate, and filtered. The filtrate was rotary-evaporated to dryness. The crude product was stirred with methyl tert-butyl ether (10 mL), and filtered. The filter cake was rinsed with methyl tert-butyl ether (1 mL×3), collected and rotary-evaporated to dryness to afford compound 6.

¹H NMR (400 MHz, CDCl₃) δ 8.28 (d, J=1.6 Hz, 1H), 8.05 (s, 1H), 7.73 (d, J=1.6 Hz, 1H), 7.32 (s, 1H), 7.30 (d, J=1.6 Hz, 1H), 4.49˜4.48 (m, 1H), 4.20˜4.16 (m, 1H), 4.08˜4.05 (m, 1H), 3.95˜3.92 (m, 1H), 3.85˜3.82 (m, 1H), 3.75 (s, 3H), 3.73˜3.67 (m, 1H), 3.62˜3.55 (m, 1H), 1.49 (d, J=6.8 Hz, 3H)

### Example 7

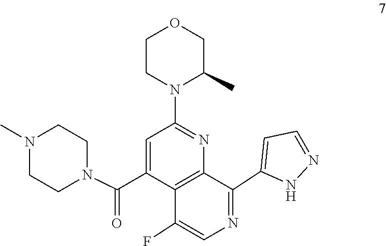 [SMILES: C[C@@H]1COCCN1c1cc(C(=O)N2CCN(C)CC2)c2c(F)cnc(-c3ccn[nH]3)c2n1]

Synthetic route:

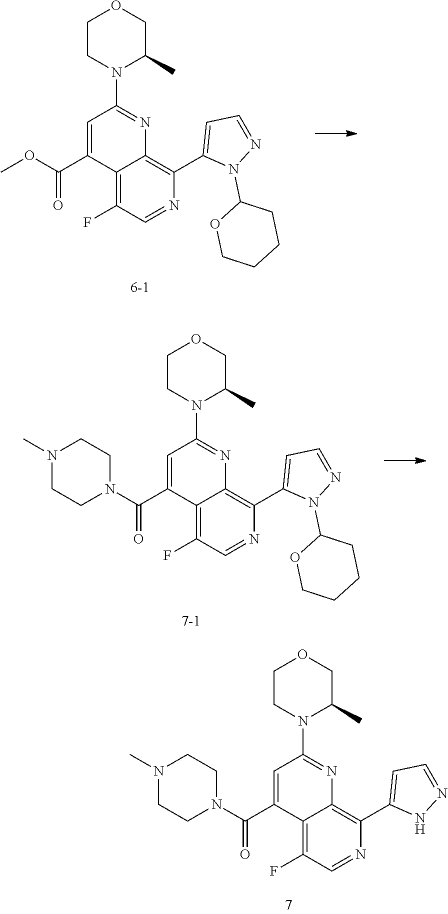 [SMILES: COC(=O)c1cc(N2CCOC[C@H]2C)nc2c(-c3ccnn3C3CCCCO3)ncc(F)c12 ; C[C@@H]1COCCN1c1cc(C(=O)N2CCN(C)CC2)c2c(F)cnc(-c3ccnn3C3CCCCO3)c2n1 ; C[C@@H]1COCCN1c1cc(C(=O)N2CCN(C)CC2)c2c(F)cnc(-c3ccn[nH]3)c2n1]

Step 1: Synthesis of Compound 7˜1

Compound 6-1 (0.1 g, 219.55 μmol) and N-methylpiperazine (43.98 mg, 439.10 μmol, 48.71 μL) were dissolved in toluene (2 mL), and trimethylaluminum (2 M, 329.32 μL) was added. The mixture was stirred at 90° C. under nitrogen for 5 hours. The reaction solution was cooled to room temperature, and quenched by slowly adding water (10 mL). The mixture was filtered. The filter cake was washed with ethyl acetate (5 mL×2), and the aqueous phase was extracted with ethyl acetate (10 mL). The organic phase was washed with saturated brine (10 mL), dried over anhydrous sodium sulfate, and filtered. The filtrate was rotary-evaporated to dryness. The crude product was purified by preparative thin layer chromatography (eluent: ethyl acetate:methanol=3:1) to afford compound 7˜1.

Step 2: Synthesis of Compound 7

Compound 7˜1 (80 mg, 152.79 μmol) was dissolved in trifluoroacetic acid (2 mL), and the mixture was stirred at room temperature for 2 hours. The reaction solution was concentrated under reduced pressure to remove the solvent. The crude product was purified by reversed-phase column (trifluoroacetic acid), and the fraction was concentrated under reduced pressure to remove most of the acetonitrile. The aqueous phase was adjusted to a pH of 7˜8 with saturated aqueous sodium bicarbonate solution. The mixture was extracted with dichloromethane (15 mL×3). The organic phase was washed with saturated saline (20 mL), dried over anhydrous sodium sulfate, and filtered. The filtrate was rotary-evaporated to dryness to afford compound 7.

¹H NMR (400 MHz, CDCl₃) δ 8.30 (s, 1H), 7.71 (d, J=2.0 Hz, 1H), 7.26 (s, 1H), 7.10 (d, J=3.2 Hz, 1H), 4.45˜4.43 (m, 1H), 4.18˜4.16 (m, 2H), 3.95˜3.92 (m, 1H), 3.83˜3.81 (m, 2H), 3.81˜3.80 (m, 1H), 3.67˜3.56 (m, 1H), 3.41˜3.32 (m, 2H), 2.75˜2.45 (m, 8H), 1.47˜1.45 (m, 3H)

### Example 8

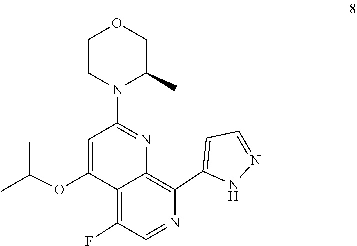 [SMILES: CC(C)Oc1cc(N2CCOC[C@H]2C)nc2c(-c3ccn[nH]3)ncc(F)c12]

Synthetic route:

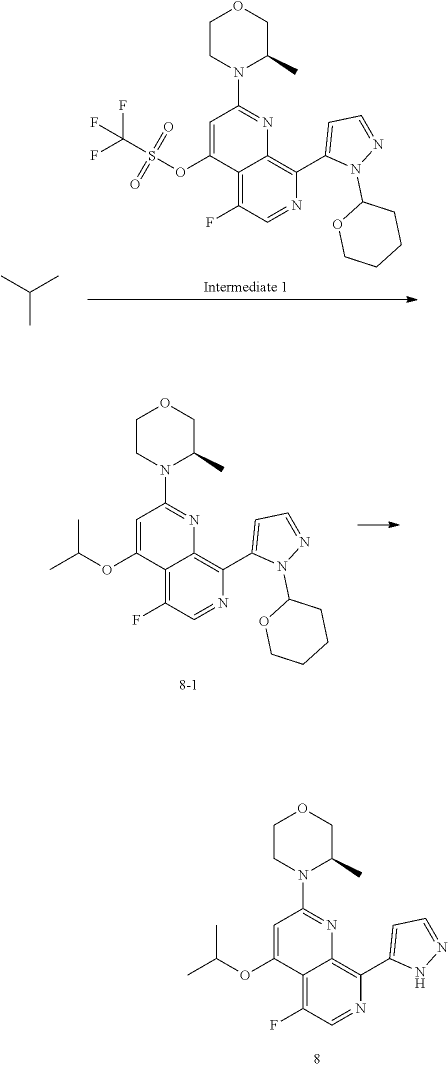

Step 1: Synthesis of Compound 8-1

Intermediate 1 (150 mg, 362.81 μmol) was dissolved in acetonitrile (6 mL), and potassium carbonate (100.28 mg, 725.61 μmol) was added. The mixture was stirred for 30 min, and then 2-iodopropane (123.35 mg, 725.61 μmol) was added. The reaction solution was stirred at 60° C. for 12 hours, and then concentrated under reduced pressure to afford a crude product. The crude product was purified by column chromatography (eluent: ethyl acetate:petroleum ether=40˜50%) to afford compound 8-1.

MS m/z: 456.1 [M+H]⁺

Step 2: Synthesis of Compound 8

Compound 8-1 (130.6 mg, 286.70 μmol) was dissolved in 4 M hydrochloric acid in dioxane (5 mL), and the mixture was stirred at 25° C. for 1 hour. The reaction solution was concentrated under reduced pressure to afford a crude product, and the crude product was adjusted to a pH of 7 with saturated sodium bicarbonate solution. The mixture was extracted with dichloromethane (20 mL×3). The organic phase was dried over anhydrous sodium sulfate, and filtered. The filtrate was concentrated under reduced pressure to afford compound 8.

MS m/z: 372.0[M+H]⁺

¹H NMR (400 MHz, DMSO-d₆) δ 13.29 (br s, 1H), 8.18 (d, J=2.5 Hz, 1H), 7.61 (br s, 1H), 7.27 (s, 1H), 6.82 (s, 1H), 5.07 (td, J=5.9, 11.8 Hz, 1H), 4.60 (br d, J=4.5 Hz, 1H), 4.17 (br d, J=12.3 Hz, 1H), 4.07˜4.01 (m, 1H), 3.87˜3.78 (m, 1H), 3.70 (dd, J=2.6, 11.4 Hz, 1H), 3.56 (dt, J=2.8, 11.8 Hz, 1H), 3.31-3.26 (m, 1H), 1.39 (d, J=5.8 Hz, 6H), 1.27 (d, J=6.8 Hz, 3H)

### Example 9| N

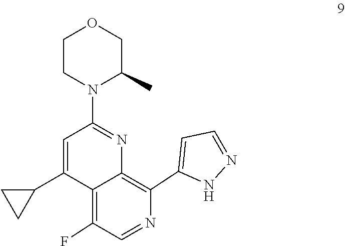 [SMILES: C[C@@H]1COCCN1c1cc(C2CC2)c2c(F)cnc(-c3ccn[nH]3)c2n1]

Synthetic route:

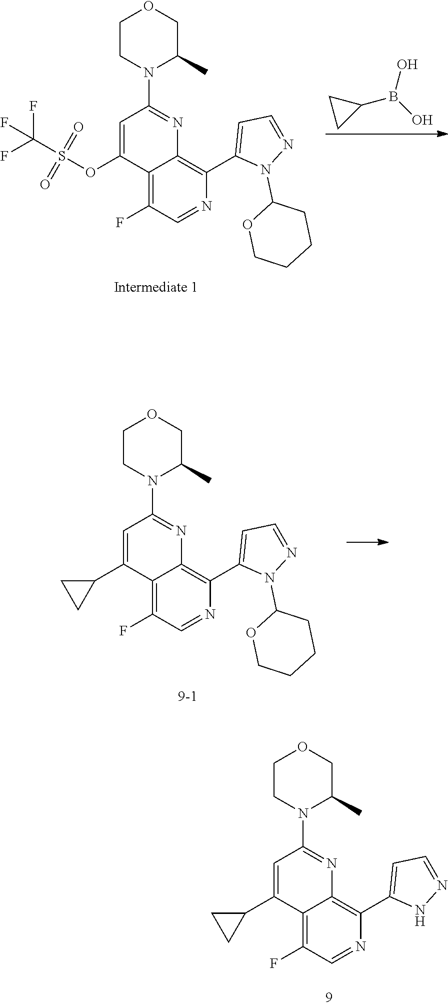 [SMILES: C[C@@H]1COCCN1c1cc(OS(=O)(=O)C(F)(F)F)c2c(F)cnc(-c3ccnn3C3CCCCO3)c2n1 ; OB(O)C1CC1 ; C[C@@H]1COCCN1c1cc(C2CC2)c2c(F)cnc(-c3ccnn3C3CCCCO3)c2n1 ; C[C@@H]1COCCN1c1cc(C2CC2)c2c(F)cnc(-c3ccn[nH]3)c2n1]

Step 1: Synthesis of Compound 9-1

Intermediate 1 (100 mg, 183.32 μmol), cyclopropylboronic acid (23.62 mg, 274.97 μmol), 1,1-bis[(diphenylphosphino)ferrocene]palladium dichloride (13.41 mg, 18.33 μmol), and sodium carbonate (38.86 mg, 366.63 μmol) were mixed with dioxane (2 mL) and water (0.2 mL). Nitrogen gas was bubbled into the system for 15 seconds, and the mixture was stirred at 100° C. in microwave for 30 minutes. The reaction solution was filtered through diatomaceous earth, and rotary-evaporated under reduced pressure to afford a crude product.

The crude product was purified by column chromatography (eluent: tetrahydrofuran:petroleum ether=0˜50%) to afford compound 9-1.

MS m/z: 438.1 [M+H]⁺

Step 2: Synthesis of Compound 9

Compound 9-1 was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was adjusted to a pH of 7 with saturated sodium bicarbonate solution. The mixture was extracted with dichloromethane (100 mL), washed with saturated brine (100 mL), dried over anhydrous sodium sulfate, and filtered.

The filtrate was rotary-evaporated under reduced pressure, then stirred with petroleum ether and ethyl acetate (petroleum ether:ethyl acetate=1:1, 3 mL), and filtered. The filter cake was dried by suction under reduced pressure to afford compound 9.

MS m/z: 354.1 [M+H]⁺

¹H NMR (400 MHz, DMSO-d₆) δ 8.30 (d, J=2.5 Hz, 1H), 7.61 (br s, 1H), 7.26 (br s, 1H), 7.08 (s, 1H), 4.62 (br d, J=5.5 Hz, 1H), 4.18 (br d, J=13.6 Hz, 1H), 4.02 (br d, J=14.1 Hz, 1H), 3.80 (br d, J=11.5 Hz, 1H), 3.68 (br d, J=9.5 Hz, 1H), 3.53 (br t, J=10.8 Hz, 1H), 2.56 (br d, J=12.0 Hz, 1H), 2.22 (m, 1H), 1.25 (d, J=6.5 Hz, 3H), 1.08-0.94 (m, 4H)

### Examples 10, 11 and 12

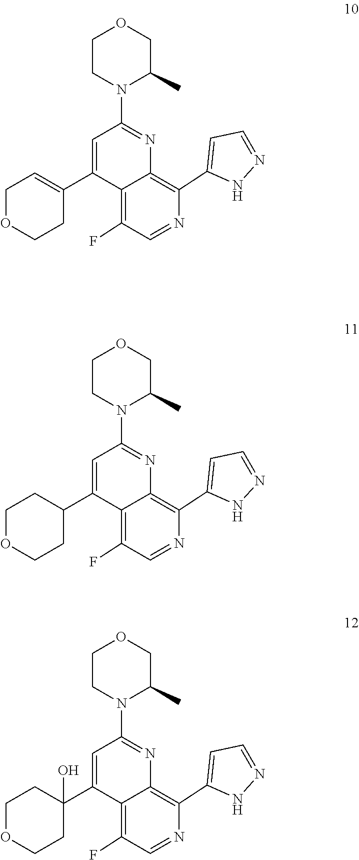 [SMILES: C[C@@H]1COCCN1c1cc(C2=CCOCC2)c2c(F)cnc(-c3ccn[nH]3)c2n1 ; C[C@@H]1COCCN1c1cc(C2CCOCC2)c2c(F)cnc(-c3ccn[nH]3)c2n1 ; C[C@@H]1COCCN1c1cc(C2(O)CCOCC2)c2c(F)cnc(-c3ccn[nH]3)c2n1]

Synthetic route:

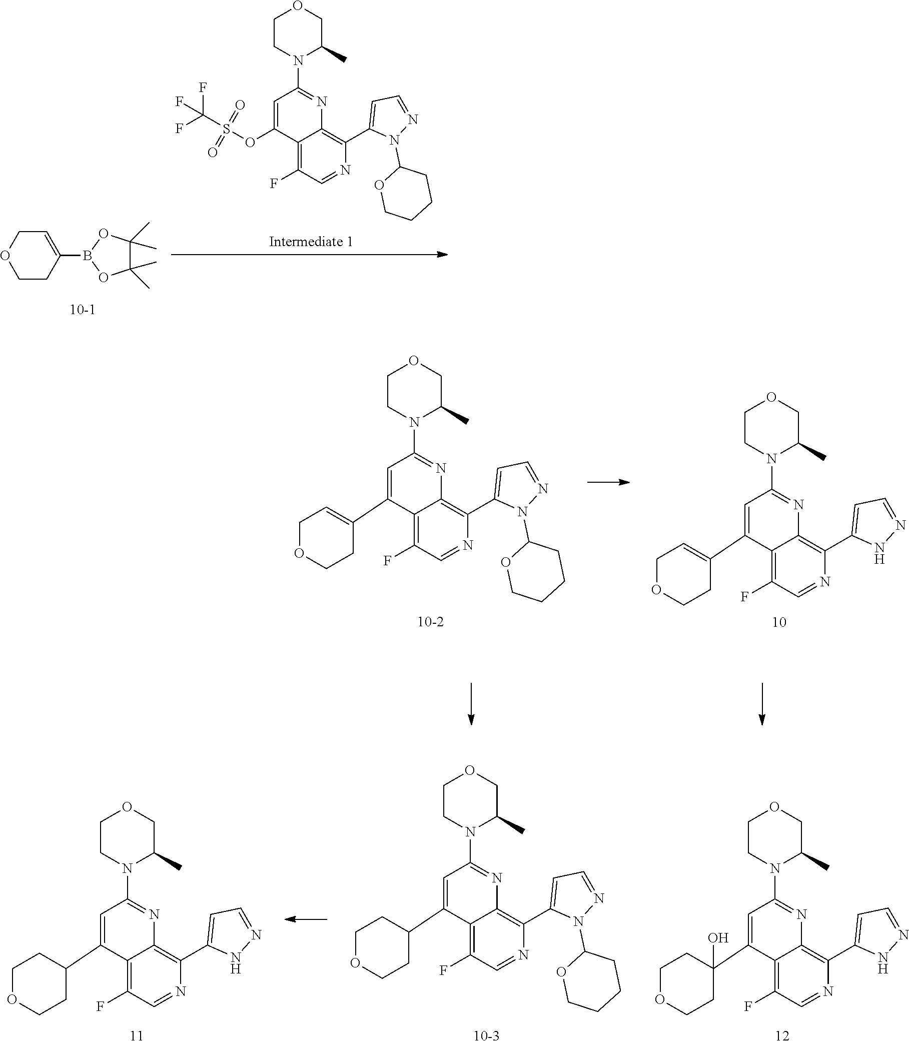

Step 1: Synthesis of Compound 10-2

Intermediate 1 (300 mg, 549.95 μmol), compound 10-1 (138.64 mg, 659.94 μmol), 1,1-bis[(diphenylphosphino)ferrocene]palladium dichloride (66 mg, 90.20 μmol), and sodium carbonate (116.58 mg, 1.10 mmol) was mixed in dioxane (5 mL) and water (0.5 mL). Nitrogen gas was bubbled into the system for 15 seconds, and the mixture was stirred at 100° C. in microwave for 1 hour. The reaction solution was filtered through diatomaceous earth, and the filtrate was concentrated under reduced pressure to afford a crude product. The crude product was purified by column chromatography (eluent: tetrahydrofuran:petroleum ether=0˜40%) to afford compound 10-2.

MS m/z: 480.0 [M+H]⁺

Step 2: Synthesis of Compound 10

Compound 10-2 (230 mg, 479.62 μmol) was dissolved in 4M hydrochloric acid in dioxane (10 mL) and methanol (2 mL). The reaction solution was stirred at 25° C. for 1 h, and then concentrated under reduced pressure to afford a crude product. The crude product was adjusted to a pH of 7 with saturated sodium bicarbonate solution. The mixture was extracted with dichloromethane (20 mL×3). The organic phase was dried over anhydrous sodium sulfate, and filtered. The filtrate was concentrated under reduced pressure to afford compound 10.

MS m/z: 396.0 [M+H]⁺

¹H NMR (400 MHz, DMSO-d₆) δ 13.32 (br s, 1H), 8.28 (s, 1H), 7.61 (br s, 1H), 7.34-7.22 (m, 2H), 5.84 (br s, 1H), 4.62 (br s, 1H), 4.29-4.15 (m, 3H), 4.03 (br d, J=7.3 Hz, 1H), 3.87 (br t, J=5.0 Hz, 2H), 3.81 (br d, J=11.3 Hz, 1H), 3.69 (br d, J=10.3 Hz, 1H), 3.60-3.49 (m, 1H), 3.40-3.35 (m, 1H), 2.34 (br s, 2H), 1.29 (br d, J=6.5 Hz, 3H)

Step 3: Synthesis of Compound 10-3

Compound 10-2 (100 mg, 208.53 mmol) was dissolved in tetrahydrofuran (10 mL), and Pd/C (10 mg, 10% purity) was added. The air in the reaction system was replaced 3 times with hydrogen, and the mixture was stirred at 25° C. for 12 hours. The reaction solution was filtered through diatomaceous earth, and the filtrate was concentrated under reduced pressure to afford a crude product. The crude product was purified by column chromatography (eluent: tetrahydrofuran:dichloromethane=0˜10%) to afford compound 10-3.

MS m/z: 481.9[M+H]⁺

Step 4: Synthesis of Compound 11

Compound 10-3 (60 mg, 124.59 μmol) was dissolved in 4M hydrochloric acid in dioxane (10 mL). The reaction solution was stirred at 40° C. for 2 h, and then concentrated under reduced pressure to afford a crude product. The crude product was purified by neutral preparative high performance liquid chromatography to afford compound 11.

MS m/z: 397.9 [M+H]⁺

¹H NMR (400 MHz, DMSO-d₆) δ 13.38-13.24 (m, 1H), 8.36-8.22 (m, 1H), 7.60 (br s, 1H), 7.38-7.19 (m, 2H), 4.65 (br s, 1H), 4.29-4.16 (m, 2H), 4.03 (br d, J=9.5 Hz, 3H), 3.91-3.79 (m, 2H), 3.76-3.47 (m, 6H), 3.43-3.36 (m, 2H), 1.31-1.26 (m, 3H)

Step 5: Synthesis of Compound 12

Compound 10 (130 mg, 328.76 μmol) was dissolved in isopropanol (9 mL) and dichloromethane (1 mL), and the mixture was cooled down to 0° C. tris(2,2,6,6-tetramethyl-3,5-heptanedionato)manganese (19.88 mg, 32.88 μmol) and phenylsilane (71.15 mg, 657.51 μmol) were added. The air in the reaction system was replaced three times with oxygen, and the mixture was stirred at 25° C. for 3 hours. The reaction solution was filtered through diatomaceous earth, and the filtrate was concentrated under reduced pressure to afford a crude product. The crude product was purified by neutral preparative high performance liquid chromatography to afford compound 12.

MS m/z: 414.0 [M+H]⁺

¹H NMR (400 MHz, CDCl₃) δ 8.35 (d, J=4.5 Hz, 1H), 7.69 (d, J=1.8 Hz, 1H), 7.57 (s, 1H), 7.22 (d, J=1.8 Hz, 1H), 4.46 (br d, J=4.8 Hz, 1H), 4.22-4.14 (m, 1H), 4.10-3.90 (m, 6H), 3.82 (dd, J=2.9, 11.4 Hz, 1H), 3.68 (dt, J=2.6, 11.7 Hz, 1H), 3.62-3.51 (m, 1H), 3.13 (br s, 1H), 2.47-2.34 (m, 2H), 1.90 (br d, J=14.6 Hz, 2H), 1.46 (d, J=6.8 Hz, 3H)

### Example 13

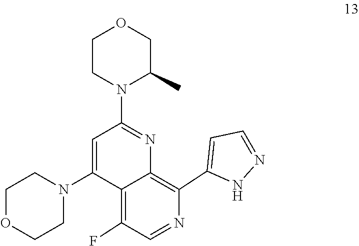 [SMILES: C[C@@H]1COCCN1c1cc(N2CCOCC2)c2c(F)cnc(-c3ccn[nH]3)c2n1]

Synthetic route:

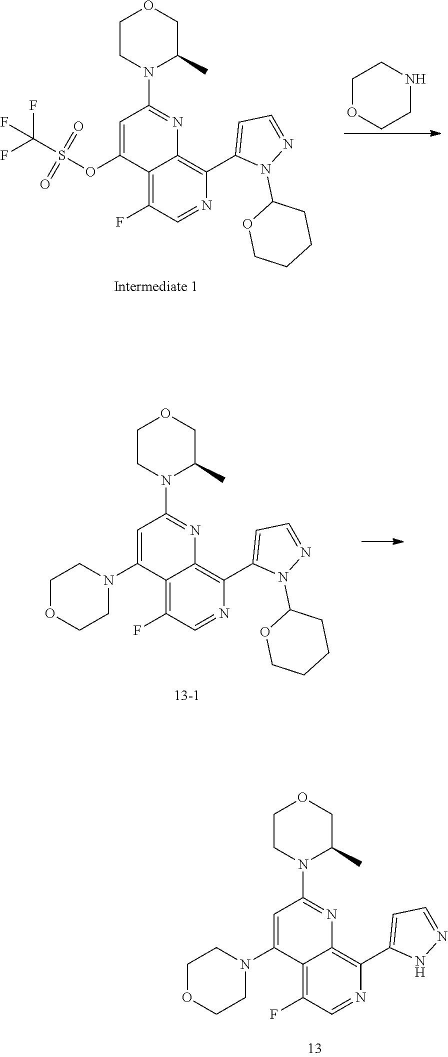 [SMILES: C[C@@H]1COCCN1c1cc(OS(=O)(=O)C(F)(F)F)c2c(F)cnc(-c3ccnn3C3CCCCO3)c2n1 ; C1COCCN1 ; C[C@@H]1COCCN1c1cc(N2CCOCC2)c2c(F)cnc(-c3ccnn3C3CCCCO3)c2n1 ; C[C@@H]1COCCN1c1cc(N2CCOCC2)c2c(F)cnc(-c3ccn[nH]3)c2n1]

Step 1: Synthesis of Compound 13-1

Morpholine (95.82 mg, 1.10 mmol), intermediate 1 (100 mg, 183.32 μmol), bis(dibenzylideneacetone) palladium (10.54 mg, 18.33 μmol), 2-(di-tert-butylphosphino)biphenyl (10.94 mg, 36.66 μmol) and potassium phosphate (116.74 mg, 549.95 μmol) were mixed in ethylene glycol dimethyl ether (5 mL). Nitrogen gas was bubbled into the system for 20 seconds, and then the mixture was stirred at 90° C. for 12 hours.

The reaction solution was filtered through diatomaceous earth, and the filtrate was rotary-evaporated under reduced pressure to afford a crude product. The crude product was purified by column chromatography (eluent: tetrahydrofuran:petroleum ether=0˜25%˜50%) to afford compound 13-1.

MS m/z: 483.2 [M+H]⁺

Step 2: Synthesis of Compound 13

Compound 13-1 (20 mg, 41.45 μmol) was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was purified by neutral preparative high performance liquid chromatography to afford compound 13.

MS m/z: 399.1 [M+H]⁺

¹H NMR (400 MHz, CHLOROFORM-d) δ 8.22 (d, J=3.0 Hz, 1H), 7.71 (d, J=2.0 Hz, 1H), 7.20 (d, J=1.5 Hz, 1H), 6.47 (s, 1H), 4.41 (br d, J=4.0 Hz, 1H), 4.23-4.17 (m, 1H), 3.98 (t, J=4.5 Hz, 5H), 3.94 (br s, 1H), 3.89-3.83 (m, 1H), 3.71 (dt, J=3.0, 11.8 Hz, 1H), 3.61-3.51 (m, 1H), 3.20 (br s, 4H), 1.47 (d, J=7.0 Hz, 3H)

### Example 14

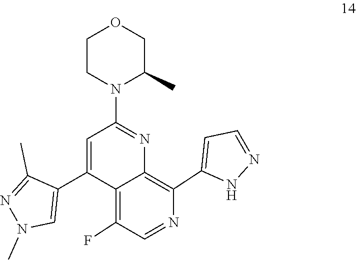 [SMILES: Cc1nn(C)cc1-c1cc(N2CCOC[C@H]2C)nc2c(-c3ccn[nH]3)ncc(F)c12]

Synthetic route:

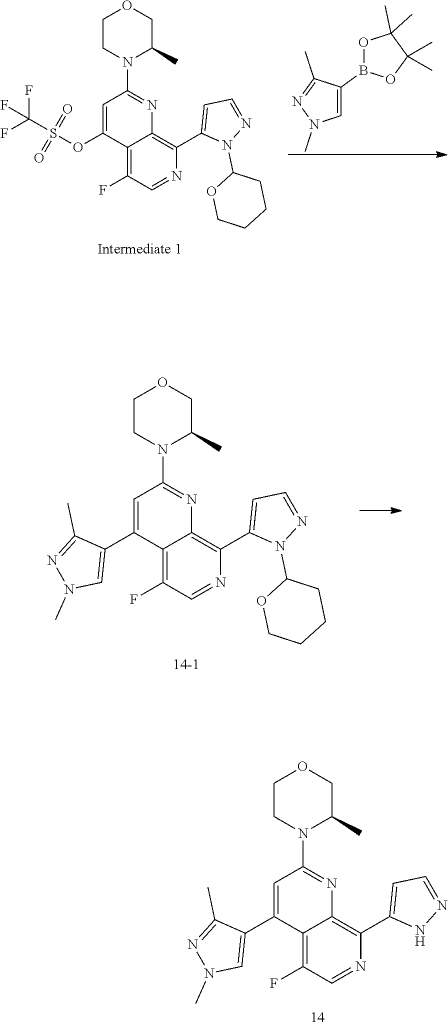 [SMILES: C[C@@H]1COCCN1c1cc(OS(=O)(=O)C(F)(F)F)c2c(F)cnc(-c3ccnn3C3CCCCO3)c2n1 ; Cc1nn(C)cc1B1OC(C)(C)C(C)(C)O1 ; Cc1nn(C)cc1-c1cc(N2CCOC[C@H]2C)nc2c(-c3ccnn3C3CCCCO3)ncc(F)c12 ; Cc1nn(C)cc1-c1cc(N2CCOC[C@H]2C)nc2c(-c3ccn[nH]3)ncc(F)c12]

Step 1: Synthesis of Compound 14-1

1.3-Dimethyl-1H-pyrazole-4-boronic acid pinacol ester (52.93 mg, 238.31 μmol), intermediate 1 (100 mg, 183.32 μmol), 1,1-bis[(diphenylphosphino)ferrocene]palladium dichloride (13.41 mg, 18.33 μmol), sodium carbonate (48.57 mg, 458.29 μmol), dioxane (2 mL) and water (0.2 mL) were mixed. Nitrogen gas was bubbled into the system for 15 seconds, and then the mixture was stirred at 100° C. in microwave for 30 minutes. The reaction solution was filtered through diatomaceous earth, and the filtrate was rotary-evaporated under reduced pressure to afford a crude product. The crude product was purified by column chromatography (eluent: ethyl acetate:petroleum ether=0˜25%˜50%) to afford compound 14-1.

MS m/z: 492.2 [M+H]⁺

Step 2: Synthesis of Compound 14

Compound 14-1 (70 mg, 142.40 μmol) was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was adjusted to a pH of 7 with saturated sodium bicarbonate solution. The mixture was extracted with dichloromethane (100 mL), washed with saturated brine (50 mL), dried over anhydrous sodium sulfate, and filtered. The filtrate was rotary evaporated under reduced pressure to afford a crude product. The crude product was stirred with petroleum ether and ethyl acetate (petroleum ether:ethyl acetate=1:1, 3 mL), and filtered. The filter cake was dried by suction under reduced pressure to afford compound 14.

MS m/z: 408.1 [M+H]⁺

¹H NMR (400 MHz, DMSO-d₆) δ 8.23 (d, J=2.0 Hz, 1H), 7.85 (s, 1H), 7.63 (s, 1H), 7.32-7.25 (m, 2H), 4.60 (br d, J=5.0 Hz, 1H), 4.20 (br d, J=13.1 Hz, 1H), 4.03 (br d, J=7.5 Hz, 1H), 3.84 (s, 3H), 3.80 (br d, J=11.5 Hz, 1H), 3.69 (br d, J=9.5 Hz, 1H), 3.59-3.51 (m, 1H), 3.31 (br s, 1H), 2.06 (s, 3H), 1.28 (d, J=7.0 Hz, 3H)

### Example 15

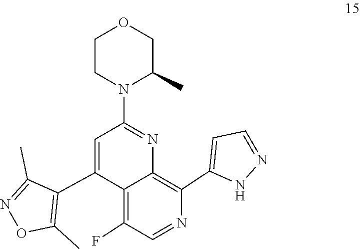 [SMILES: Cc1noc(C)c1-c1cc(N2CCOC[C@H]2C)nc2c(-c3ccn[nH]3)ncc(F)c12]

Synthetic route:

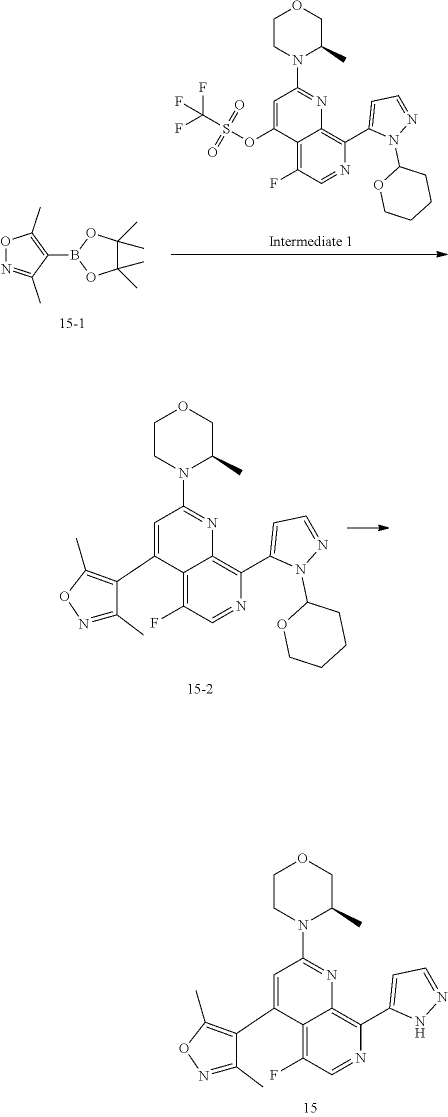 [SMILES: Cc1noc(C)c1B1OC(C)(C)C(C)(C)O1 ; C[C@@H]1COCCN1c1cc(OS(=O)(=O)C(F)(F)F)c2c(F)cnc(-c3ccnn3C3CCCCO3)c2n1 ; Cc1noc(C)c1-c1cc(N2CCOC[C@H]2C)nc2c(-c3ccnn3C3CCCCO3)ncc(F)c12 ; Cc1noc(C)c1-c1cc(N2CCOC[C@H]2C)nc2c(-c3ccn[nH]3)ncc(F)c12]

Step 1: Synthesis of Compound 15-2

Intermediate 1 (50 mg, 91.66 μmol), 15-1 (26.58 mg, 119.16 μmol), 1,1-bis[(diphenylphosphino)ferrocene]palladium dichloride (11 mg, 15.03 μmol), and sodium carbonate (23 mg, 217.00 μmol) were mixed in dioxane (2 mL) and water (0.2 mL). Nitrogen gas was bubbled into the system for 15 seconds, and then the mixture was stirred at 100° C. in microwave for 1 h. The reaction solution was filtered through diatomaceous earth, and the filtrate was concentrated under reduced pressure to afford a crude product. The crude product was purified by column chromatography (eluent: tetrahydrofuran:petroleum ether=30˜40%) to afford compound 15-2.

MS m/z: 493.2 [M+H]⁺

Step 2: Synthesis of Compound 15

Compound 15-2 (40 mg, 81.21 μmol) was dissolved in 4M hydrochloric acid in dioxane (5 mL) and methanol (1 mL). The reaction solution was stirred at 25° C. for 1 h, and then concentrated under reduced pressure to afford a crude product. The crude product was adjusted to a pH of 7 with saturated sodium bicarbonate solution. The mixture was extracted with dichloromethane (10 mL×3). The organic phase was dried over anhydrous sodium sulfate, and filtered. The filtrate was concentrated under reduced pressure to afford a crude product. Ethyl acetate (50 μL) and petroleum ether (50 μL) were added to the crude product, and the mixture was stirred for 10 minutes. The mixture was filtered. The filter cake was collected, and dried to afford compound 15.

MS m/z: 409.0 [M+H]⁺

¹H NMR (400 MHz, DMSO-d₆) δ 13.38 (br s, 1H), 8.28 (s, 1H), 7.63 (br s, 1H), 7.49 (s, 1H), 7.35 (s, 1H), 4.63 (br s, 1H), 4.25 (br d, J=12.8 Hz, 1H), 4.04 (brt, J=7.0 Hz, 1H), 3.82 (br d, J=11.0 Hz, 1H), 3.71 (br d, J=12.0 Hz, 1H), 3.56 (br d, J=11.8 Hz, 2H), 2.34 (s, 3H), 2.08 (s, 3H), 1.30 (br d, J=6.5 Hz, 3H)

### Example 16

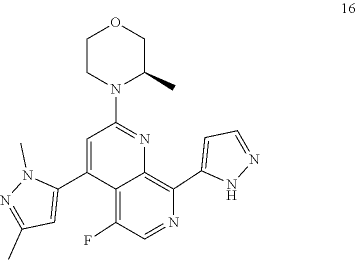 [SMILES: Cc1cc(-c2cc(N3CCOC[C@H]3C)nc3c(-c4ccn[nH]4)ncc(F)c23)n(C)n1]

Synthetic route:

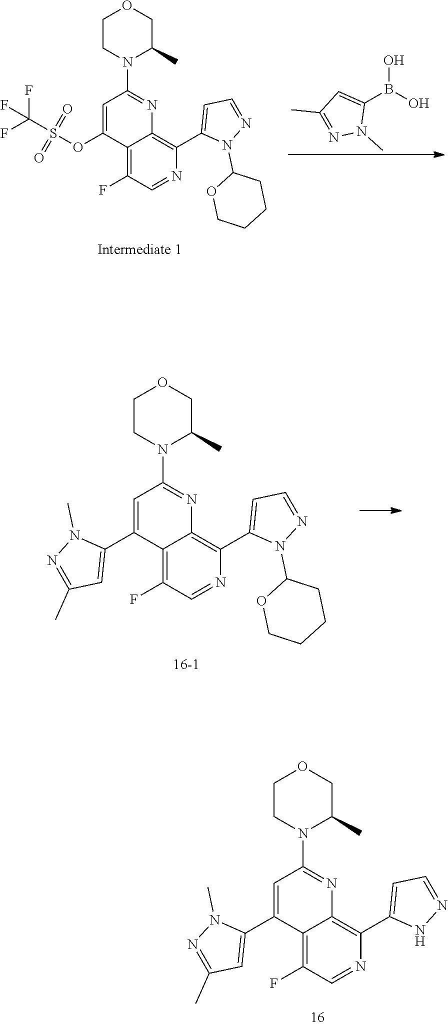

Step 1: Synthesis of Compound 16-1

(1,3-Dimethyl-1H-pyrazol-5-yl)-boronic acid (33.35 mg, 238.32 μmol), intermediate 1 (100 mg, 183.32 μmol), 1,1-bis[(diphenylphosphino)ferrocene]palladium dichloride (13.41 mg, 18.33 μmol), and sodium carbonate (3.89 mg, 36.66 μmol) were mixed in dioxane (2 mL) and water (0.2 mL). Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product. The crude product was purified by column chromatography (eluent: tetrahydrofuran:petroleum ether=0˜50%) to afford compound 16-1.

MS m/z: 492.1 [M+H]⁺

Step 2: Synthesis of Compound 16

Compound 16-1 (59 mg, 120.03 μmol) was dissolved in 4M hydrochloric acid in dioxane (5 mL) and ethanol (5 mL). The reaction solution was stirred at 15° C. for 30 min, and then rotary-evaporated under reduced pressure to afford a crude product. The crude product was adjusted to a pH of 7 with saturated sodium bicarbonate solution. The mixture was extracted with dichloromethane (100 mL), washed with saturated brine (50 mL), dried over anhydrous sodium sulfate, and filtered. The filtrate was rotary evaporated under reduced pressure, and then purified by neutral preparative high performance liquid chromatography to afford compound 16.

MS m/z: 408.1 [M+H]⁺

¹H NMR (400 MHz, CHLOROFORM-d) δ 8.17 (d, J=1.5 Hz, 1H), 7.64 (s, 1H), 7.19 (s, 1H), 7.05 (s, 1H), 6.09 (s, 1H), 4.36 (br d, J=5.5 Hz, 1H), 4.10 (dd, J=3.5, 11.5 Hz, 1H), 3.98 (br d, J=12.5 Hz, 1H), 3.88-3.83 (m, 1H), 3.80-3.73 (m, 1H), 3.63 (dt, J=3.0, 11.8 Hz, 1H), 3.53 (s, 3H), 3.51-3.44 (m, 1H), 2.28 (s, 3H), 1.40 (d, J=6.5 Hz, 3H)

### Example 17

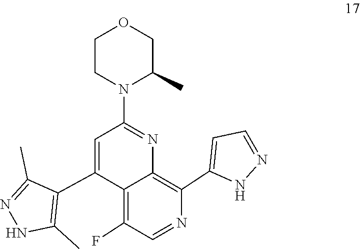 [SMILES: Cc1n[nH]c(C)c1-c1cc(N2CCOC[C@H]2C)nc2c(-c3ccn[nH]3)ncc(F)c12]

Synthetic route:

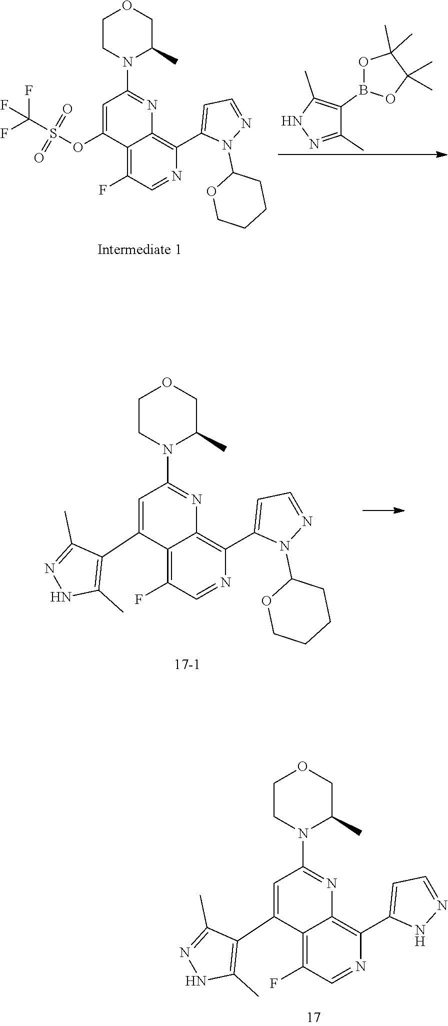 [SMILES: C[C@@H]1COCCN1c1cc(OS(=O)(=O)C(F)(F)F)c2c(F)cnc(-c3ccnn3C3CCCCO3)c2n1 ; Cc1n[nH]c(C)c1B1OC(C)(C)C(C)(C)O1 ; Cc1n[nH]c(C)c1-c1cc(N2CCOC[C@H]2C)nc2c(-c3ccnn3C3CCCCO3)ncc(F)c12 ; Cc1n[nH]c(C)c1-c1cc(N2CCOC[C@H]2C)nc2c(-c3ccn[nH]3)ncc(F)c12]

Step 1: Synthesis of Compound 17-1

Intermediate 1 (100 mg, 183.32 μmol), 3,5-dimethylpyrazole-4-boronic acid pinacol ester (62.07 mg, 219.98 μmol), 1,1-bis[(diphenylphosphino)ferrocene]palladium dichloride (181.07 mg, 247.47 μmol), and sodium carbonate (38.86 mg, 366.63 μmol) were mixed in dioxane (2 mL) and water (0.2 mL). Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product. The crude product was purified by column chromatography (eluent: tetrahydrofuran:petroleum ether=0˜50%) to afford compound 17-1.

MS m/z: 492.1 [M+H]⁺

Step 2: Synthesis of Compound 17

Compound 17-1 (70 mg, 142.40 μmol) was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was adjusted to a pH of 8 with saturated sodium bicarbonate solution. The mixture was extracted with dichloromethane (100 mL), washed with saturated brine (100 mL), dried over anhydrous sodium sulfate, and filtered. The filtrate was rotary evaporated under reduced pressure, then stirred with petroleum ether and ethyl acetate (petroleum ether:ethyl acetate=1:1, 3 mL), and filtered. The solid was dried by suction under reduced pressure to afford compound 17.

MS m/z: 408.1 [M+H]⁺

¹H NMR (400 MHz, DMSO-d₆) δ 8.28 (s, 1H), 7.63 (br s, 2H), 7.49 (s, 1H), 7.35 (s, 1H), 4.63 (br s, 1H), 4.25 (br d, J=12.8 Hz, 1H), 4.04 (br t, J=7.0 Hz, 1H), 3.82 (br d, J=11.0 Hz, 1H), 3.71 (br d, J=12.0 Hz, 1H), 3.56 (br d, J=11.8 Hz, 2H), 2.08 (br d, J=3.0 Hz, 6H), 1.29 (br d, J=6.5 Hz, 3H)

### Example 18

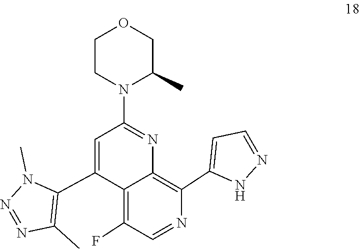 [SMILES: Cc1nnn(C)c1-c1cc(N2CCOC[C@H]2C)nc2c(-c3ccn[nH]3)ncc(F)c12]

Synthetic route:

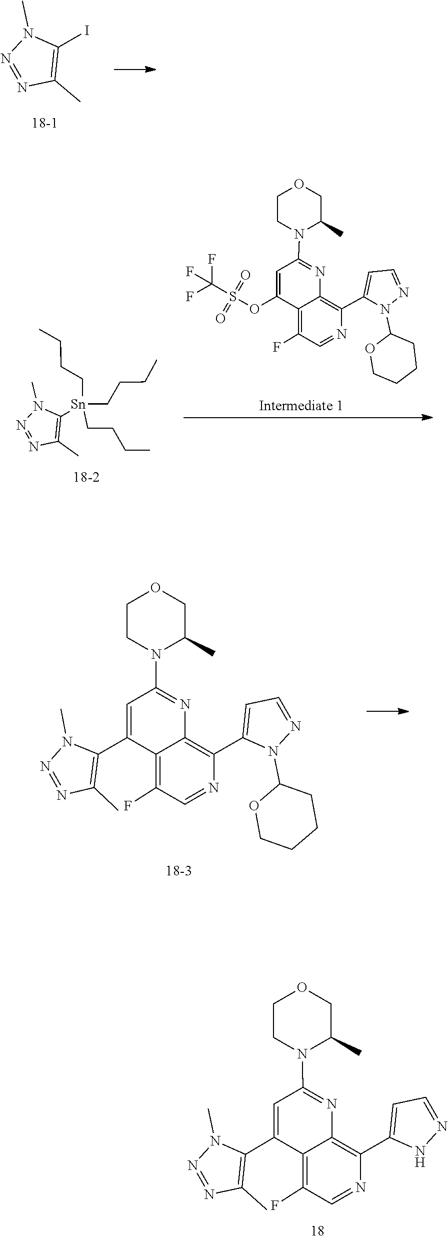

Step 1: Synthesis of Compound 18-2

Compound 18-1 (500 mg, 2.24 mmol) and bis[di-tert-butyl-(4-dimethylaminophenyl)phosphine]palladium dichloride (158.75 mg, 224.20 μmol) were added to dioxane (10 mL), and the air in the reaction system was replaced 3 times with nitrogen. Hexabutyldistannane (1.79 g, 3.09 mmol, 1.54 mL) was added. The reaction solution was stirred at 110° C. for 3 h. The reaction solution was filtered through diatomaceous earth containing potassium fluoride solid, and the filtrate was concentrated under reduced pressure to afford a crude product. The crude product was purified by column chromatography (eluent: ethyl acetate:petroleum ether=0˜50%) to afford compound 18-2.

MS m/z: 387.9 [M+H]±

Step 2: Synthesis of Compound 18-3

Intermediate 1 (50 mg, 91.66 μmol) were mixed in dioxane (10 mL), and 18-2 (70.79 mg, 183.32 μmol), tetrakis(triphenylphosphine)palladium (10.59 mg, 9.17 μmol) and lithium chloride (11.66 mg, 274.97 μmol) were added. The air in the reaction system was replaced 3 times with nitrogen, and the mixture was stirred at 100° C. for 72 hours. The reaction solution was concentrated under reduced pressure to afford a crude product. The crude product was purified by column chromatography (eluent: tetrahydrofuran:petroleum ether=0˜0.50%) to afford compound 18-3.

MS m/z: 493.2[M+H]⁺

Step 3: Synthesis of Compound 18

Compound 18-3 (45 mg, 91.36 μmol) was dissolved in dichloromethane (10 mL), and trifluoroacetic acid (5 mL) was added. The reaction solution was stirred at 25° C. for 12 hours, and then adjusted to a pH of 8 with saturated sodium carbonate solution. The mixture was extracted with dichloromethane (20 mL×4), dried over anhydrous sodium sulfate, and filtered.

The organic phase was concentrated under reduced pressure to afford a crude product. The crude product was purified by acidic preparative high performance liquid chromatography. The separated solution was concentrated under reduced pressure, and dichloromethane (10 mL) and water (5 mL) were added. The mixture was adjusted to a pH of 8 with saturated sodium carbonate solution, and the layers were separated. The aqueous phase was extracted with dichloromethane (10 mL×3). The organic phase was dried over anhydrous sodium sulfate, and filtered. The filtrate was concentrated under reduced pressure to afford compound 18.

MS m/z: 409.0 [M+H]⁺

¹H NMR (400 MHz, DMSO-d₆) δ 13.41 (br s, 1H), 8.30 (s, 1H), 7.63 (br s, 2H), 7.36 (br s, 1H), 4.63 (br s, 1H), 4.27 (br d, J=12.8 Hz, 1H), 4.06 (br d, J=9.5 Hz, 1H), 3.82 (d, J 0.8 Hz, 4H), 3.72 (br d, J=10.5 Hz, 1H), 3.57 (br t, J=12.0 Hz, 1H), 3.40 (br s, 1H), 2.15 (s, 3H), 1.31 (br d, J=6.3 Hz, 3H)

### Example 19

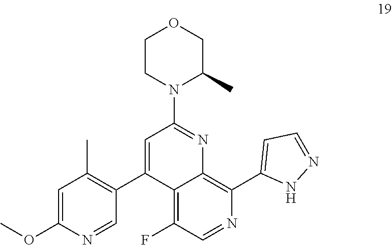 [SMILES: COc1cc(C)c(-c2cc(N3CCOC[C@H]3C)nc3c(-c4ccn[nH]4)ncc(F)c23)cn1]

Synthetic route:

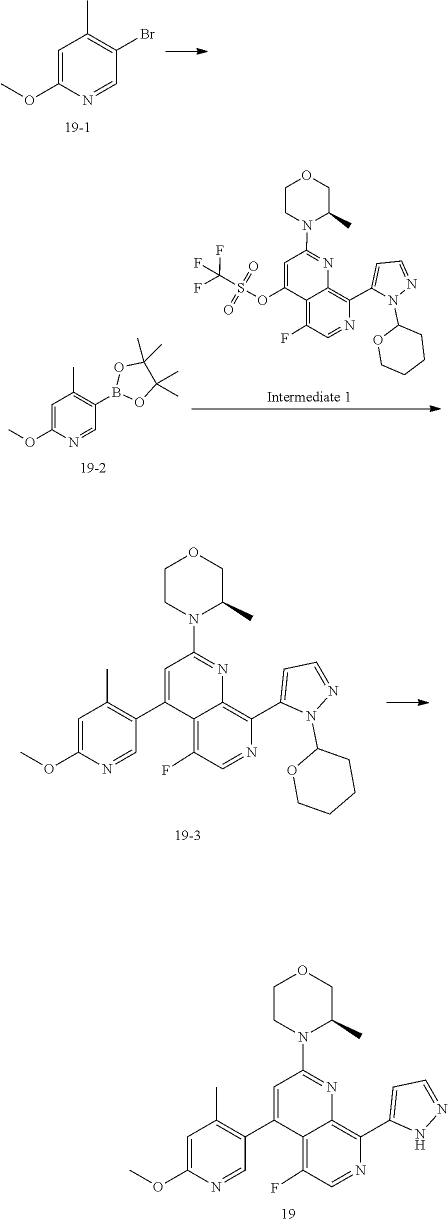

Step 1: Synthesis of Compound 19-2

Compound 19-1 (500 mg, 2.47 mmol), bis(pinacolato)diboron (1.89 g, 7.42 mmol), potassium acetate (728.61 mg, 7.42 mmol), and 1,1-bis[(diphenylphosphino)ferrocene]palladium dichloride (181.07 mg, 247.47 μmol) were mixed in dioxane (20 mL). The air in the reaction system was replaced three times with nitrogen, and the mixture was stirred at 90° C. for 3 h. The reaction solution was filtered through diatomaceous earth, and the filtrate was rotary-evaporated under reduced pressure to afford a crude product. The crude product was purified by column chromatography (eluent: ethyl acetate:petroleum ether=0˜25%) to afford compound 19-2.

MS m/z: 250.0 [M+H]⁺

Step 2: Synthesis of Compound 19-3

Intermediate 1 (50 mg, 91.66 μmol), 19-2 (27.40 mg, 109.99 μmol), 1,1-bis[(diphenylphosphino)ferrocene]palladium dichloride (6.71 mg, 9.17 μmol), and sodium carbonate (14.57 mg, 137.49 μmol) were mixed in dioxane (2 mL) and water (0.2 mL). Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product. The crude product was purified by column chromatography (eluent: ethyl acetate:petroleum ether=0˜25%) to afford compound 19-3.

MS m/z: 519.1 [M+H]⁺

Step 3: Synthesis of Compound 19

Compound 19-3 (30 mg, 57.85 μmol) was dissolved in 4M hydrochloric acid in dioxane (3 mL) and ethanol (1 mL). The reaction solution was stirred at 15° C. for 1 h, and then rotary-evaporated under reduced pressure to afford a crude product. The crude product was adjusted to a pH of 7 with saturated sodium bicarbonate solution. The mixture was extracted with dichloromethane (100 mL), washed with saturated brine (50 mL), dried over anhydrous sodium sulfate, and filtered to remove the desiccant. The filtrate was rotary evaporated under reduced pressure to afford a crude product. The crude product was purified by neutral preparative high performance liquid chromatography to afford compound 19.

MS m/z: 435.1 [M+H]⁺

¹H NMR (400 MHz, DMSO-d₆) δ 8.22 (d, J=2.0 Hz, 1H), 8.06 (s, 1H), 7.65 (br s, 1H), 7.39 (d, J=3.5 Hz, 1H), 7.34 (br s, 1H), 6.83 (s, 1H), 4.64 (br s, 1H), 4.25 (br d, J=12.5 Hz, 1H), 4.13-4.07 (m, 1H), 4.03 (d, J=7.0 Hz, 1H), 3.90 (s, 3H), 3.84-3.79 (m, 1H), 3.63-3.58 (m, 2H), 2.68, (s, 3H), 1.24 (br s, 3H)

### Example 20

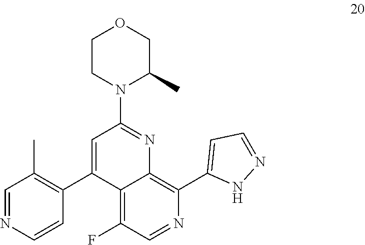 [SMILES: Cc1cnccc1-c1cc(N2CCOC[C@H]2C)nc2c(-c3ccn[nH]3)ncc(F)c12]

Synthetic route:

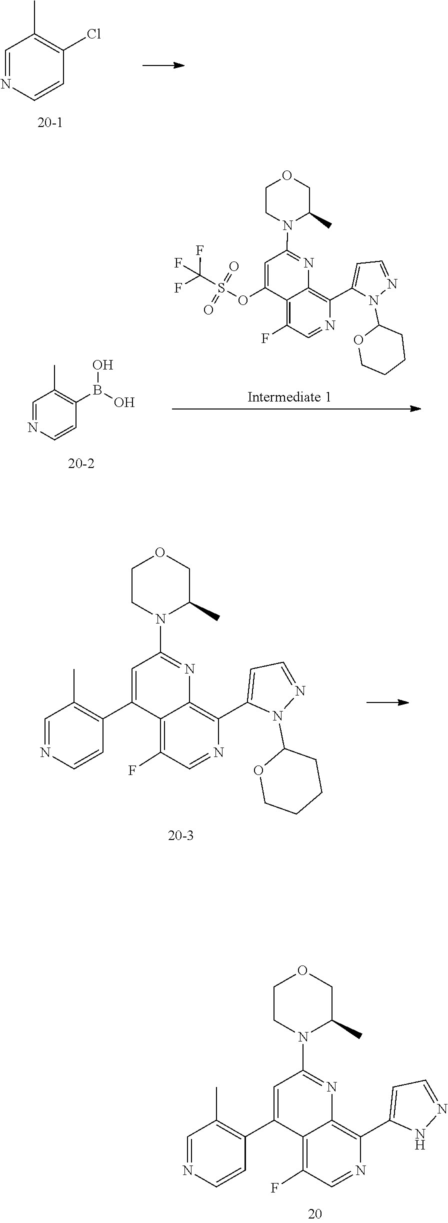

Step 1: Synthesis of Compound 20-2

Compound 20-1 (500 mg, 3.92 mmol), bis(pinacolato)diboron (1.99 g, 7.84 mmol), 1,1-bis[(diphenylphosphino)ferrocene]palladium dichloride (286.78 mg, 391.94 μmol), 2-dicyclohexylphosphino-2,4,6-triisopropylbiphenyl (373.69 mg, 783.88 μmol) and potassium acetate (1.15 g, 11.76 mmol) were mixed in dioxane (20 mL), and air in the reaction system was replaced 3 times with nitrogen. The mixture was stirred at 80° C. for 12 hours. The reaction solution was filtered through diatomaceous earth, and the filtrate was concentrated under reduced pressure to afford crude compound 20-2.

MS m/z: 137.8 [M+H]⁺

Step 2: Synthesis of Compound 20-3

Compound 20-2 (120.49 mg, 879.85 μmol), intermediate 1 (100 mg, 183.32 μmol), 1,1-bis[(diphenylphosphino)ferrocene]palladium dichloride (13.41 mg, 18.33 μmol), and sodium carbonate (58.29 mg, 549.95 μmol) were mixed in dioxane (2 mL) and water (0.2 mL).

Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product. The crude product was purified by column chromatography (eluent: tetrahydrofuran:petroleum ether=0˜25%˜50%) to afford compound 20-3.

MS m/z: 489.1[M+H]⁺

Step 3: Synthesis of Compound 20

Compound 20-3 (50 mg, 102.34 μmol) was dissolved in dichloromethane (3 mL) and trifluoroacetic acid (6 mL). The reaction solution was stirred at 15° C. for 12 h, and then adjusted to a pH of 7 with saturated sodium bicarbonate solution. The mixture was extracted with dichloromethane (100 mL), washed with saturated brine (100 mL), dried over anhydrous sodium sulfate, and filtered to remove the desiccant. The filtrate was rotary evaporated under reduced pressure to afford a crude product. The crude product was purified by neutral preparative high performance liquid chromatography to afford compound 20.

MS m/z: 405.1 [M+H]⁺

¹H NMR (400 MHz, CHLOROFORM-d) δ 8.60 (d, J=7.5 Hz, 1H), 8.21 (d, J=2.0 Hz, 1H), 7.75 (d, J=1.5 Hz, 1H), 7.77-7.72 (m, 1H), 7.30 (d, J=1.5 Hz, 1H), 7.21 (d, J=5.0 Hz, 1H), 6.98 (s, 1H), 4.45 (br d, J=7.0 Hz, 1H), 4.20 (dd, J=3.8, 11.3 Hz, 1H), 4.07 (br d, J 12.5 Hz, 1H), 3.98-3.93 (m, 1H), 3.91-3.85 (m, 1H), 3.78-3.69 (m, 1H), 3.63-3.55 (m, 1H), 2.14 (s, 3H), 1.50 (dd, J=7.0, 9.5 Hz, 3H)

### Example 21

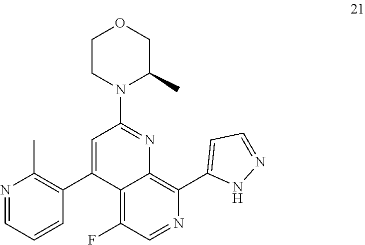 [SMILES: Cc1ncccc1-c1cc(N2CCOC[C@H]2C)nc2c(-c3ccn[nH]3)ncc(F)c12]

Synthetic route:

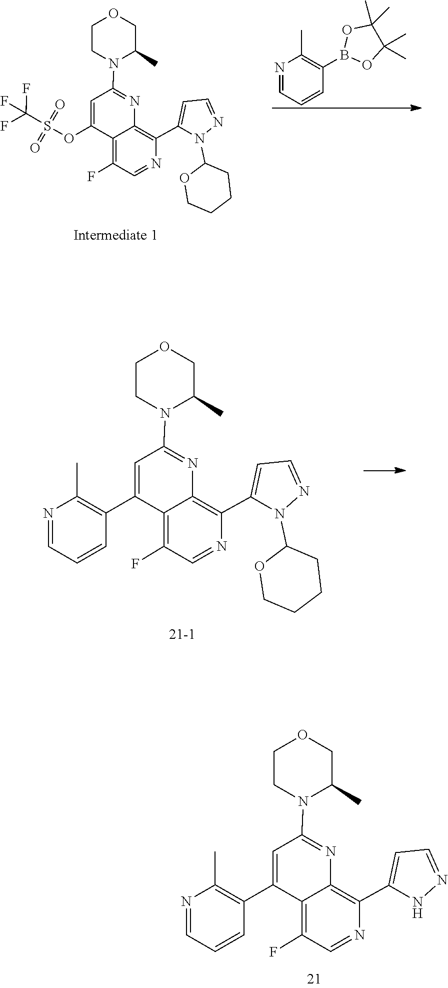 [SMILES: C[C@@H]1COCCN1c1cc(OS(=O)(=O)C(F)(F)F)c2c(F)cnc(-c3ccnn3C3CCCCO3)c2n1 ; Cc1ncccc1B1OC(C)(C)C(C)(C)O1 ; Cc1ncccc1-c1cc(N2CCOC[C@H]2C)nc2c(-c3ccnn3C3CCCCO3)ncc(F)c12 ; Cc1ncccc1-c1cc(N2CCOC[C@H]2C)nc2c(-c3ccn[nH]3)ncc(F)c12]

Step 1: Synthesis of Compound 21-1

2-Methylpyridine-3-boronic acid pinacol ester (40.16 mg, 183.32 μmol), intermediate 1 (100 mg, 183.32 μmol), 1,1-bis[(diphenylphosphino)ferrocene]palladium dichloride (13.41 mg, 18.33 μmol), and sodium carbonate (58.29 mg, 549.95 μmol) were mixed in dioxane (2 mL) and water (0.2 mL). Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product. The crude product was purified by column chromatography (eluent: tetrahydrofuran:petroleum ether=0˜25%˜50%) to afford compound 21-1.

MS m/z: 489.1 [M+H]⁺

Step 2: Synthesis of Compound 21

Compound 21-1 (70 mg, 143.28 μmol) was dissolved in 4M hydrochloric acid in dioxane (10 mL) and ethanol (2 mL). The reaction solution was stirred at 15° C. for 30 min, and then rotary-evaporated under reduced pressure to afford a crude product. The crude product was adjusted to a pH of 7 with saturated sodium bicarbonate solution. The mixture was extracted with dichloromethane (100 mL), washed with saturated brine (100 mL), dried over anhydrous sodium sulfate, and filtered. The filtrate was rotary-evaporated under reduced pressure, and then purified by column chromatography (eluent: methanol:dichloromethane=0˜10%) to afford compound 21.

MS m/z: 405.1 [M+H]⁺

¹H NMR (400 MHz, CHLOROFORM-d) δ 8.55 (dd, J=1.5, 5.0 Hz, 1H), 8.11 (d, J 2.0 Hz, 1H), 7.64 (d, J=1.5 Hz, 1H), 7.46 (td, J=2.0, 7.5 Hz, 1H), 7.22-7.18 (m, 2H), 6.93 (s, 1H), 4.36 (br d, J=6.5 Hz, 1H), 4.10 (dd, J=3.3, 11.3 Hz, 1H), 3.98 (br d, J=12.5 Hz, 1H), 3.88-3.81 (m, 1H), 3.81-3.75 (m, 1H), 3.69-3.59 (m, 1H), 3.49 (dt, J=2.3, 12.4 Hz, 1H), 2.28 (s, 3H), 1.40 (dd, J=7.0, 8.5 Hz, 3H)

### Example 22

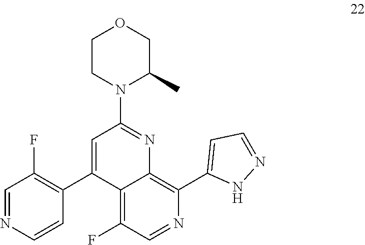 [SMILES: C[C@@H]1COCCN1c1cc(-c2ccncc2F)c2c(F)cnc(-c3ccn[nH]3)c2n1]

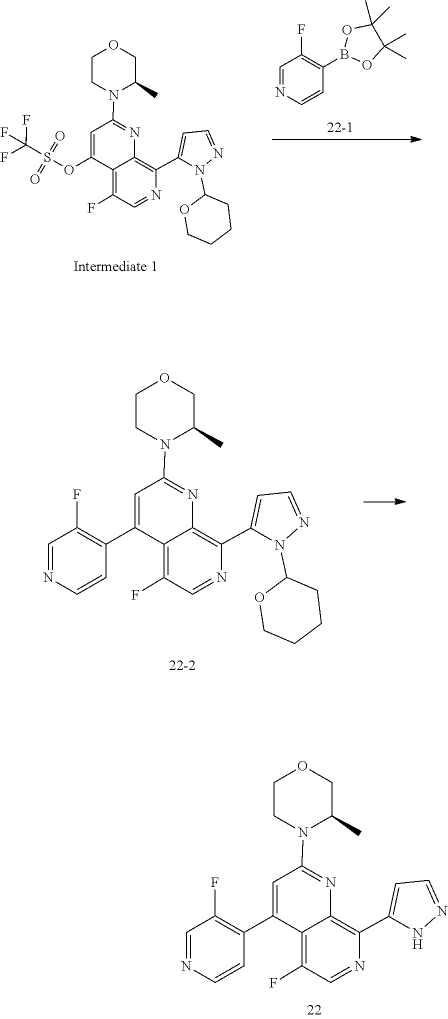 [SMILES: C[C@@H]1COCCN1c1cc(OS(=O)(=O)C(F)(F)F)c2c(F)cnc(-c3ccnn3C3CCCCO3)c2n1 ; CC1(C)OB(c2ccncc2F)OC1(C)C ; C[C@@H]1COCCN1c1cc(-c2ccncc2F)c2c(F)cnc(-c3ccnn3C3CCCCO3)c2n1 ; C[C@@H]1COCCN1c1cc(-c2ccncc2F)c2c(F)cnc(-c3ccn[nH]3)c2n1]

Step 1: Synthesis of Compound 22-2

Compound 22-1 (53.16 mg, 238.31 μmol), intermediate 1 (100 mg, 183.32 μmol), 1,1-bis[(diphenylphosphino)ferrocene]palladium dichloride (13.41 mg, 18.33 μmol), and sodium carbonate (38.86 mg, 366.63 μmol) were mixed in dioxane (2 mL) and water (0.2 mL).

Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product.

The crude product was purified by column chromatography (eluent: tetrahydrofuran:petroleum ether=0˜25%˜50%) to afford compound 22-2.

MS m/z: 493.1 [M+H]⁺

Step 2: Synthesis of Compound 22

Compound 22-2 (70 mg, 142.13 μmol) was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was adjusted to a pH of 7 with saturated sodium bicarbonate solution. The mixture was extracted with dichloromethane (100 mL), washed with saturated brine (50 mL), dried over anhydrous sodium sulfate, and filtered. The filtrate was rotary-evaporated under reduced pressure, and then purified by column chromatography (eluent: dichloromethane:methanol=20:1) to afford compound 22.

MS m/z: 409.1 [M+H]⁺

¹H NMR (400 MHz, DMSO-d₆) δ 13.40 (br s, 1H), 8.73 (s, 1H), 8.61 (d, J=5.0 Hz, 1H), 8.28 (d, J=1.5 Hz, 1H), 7.70 (br t, J=4.5 Hz, 1H), 7.64 (br s, 1H), 7.59 (br s, 1H), 7.35 (br s, 1H), 4.25 (br d, J=14.1 Hz, 1H), 4.07˜4.02 (m, 1H), 3.85-3.79 (m, 1H), 3.70 (br d, J 12.0 Hz, 1H), 3.61-3.49 (m, 2H), 3.44-3.37 (m, 1H), 1.32 (br dd, J=7.0, 11.5 Hz, 3H)

### Example 23

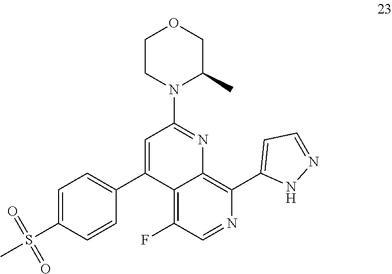 [SMILES: C[C@@H]1COCCN1c1cc(-c2ccc(S(C)(=O)=O)cc2)c2c(F)cnc(-c3ccn[nH]3)c2n1]

Synthetic route:

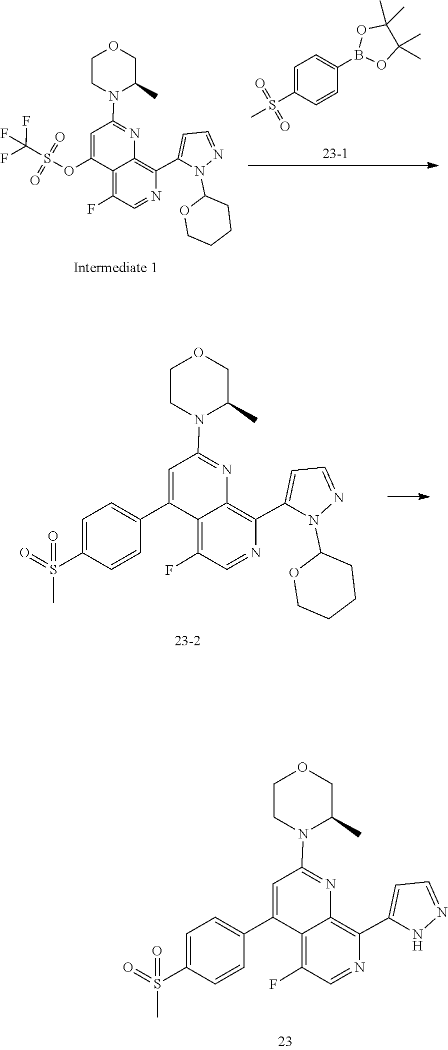

Step 1: Synthesis of Compound 23-2

Intermediate 1 (100 mg, 183.32 mmol), compound 23-1 (62.07 mg, 219.98 μmol), 1,1-bis[(diphenylphosphino)ferrocene]palladium dichloride (181.07 mg, 247.47 μmol), and sodium carbonate (38.86 mg, 366.63 μmol) were mixed in dioxane (2 mL) and water (0.2 mL).

Nitrogen gas was bubbled into the system for 15 seconds, and then the mixture was stirred at 100° C. in microwave for 3 h. The reaction solution was filtered through diatomaceous earth, and the filtrate was rotary-evaporated under reduced pressure to afford a crude product. The crude product was purified by column chromatography (eluent: tetrahydrofuran:petroleum ether=0˜50%) to afford compound 23-2.

MS m/z: 552.1[M+H]⁺

Step 2: Synthesis of Compound 23

Compound 23-2 (80 mg, 145.02 μmol) was dissolved in 4M hydrochloric acid in dioxane (10 mL) and ethanol (2 mL). The reaction solution was stirred at 20° C. for 1 h, and then rotary-evaporated under reduced pressure to afford a crude product. The crude product was adjusted to a pH of 8 with saturated sodium bicarbonate solution. The mixture was extracted with dichloromethane (100 mL), washed with saturated brine (50 mL), dried over anhydrous sodium sulfate, and filtered. The filtrate was rotary evaporated under reduced pressure, then stirred with petroleum ether and ethyl acetate (petroleum ether:ethyl acetate=1:1, 3 mL), and filtered. The filter cake was dried by suction under reduced pressure to afford compound 23.

MS m/z: 468.0 [M+H]⁺

¹H NMR (400 MHz, DMSO-d₆) δ 13.39 (br s, 1H), 8.26 (s, 1H), 8.05 (d, J=8.0 Hz, 2H), 7.83-7.78 (m, 2H), 7.64 (br s, 1H), 7.43 (s, 1H), 7.36 (br s, 1H), 4.66 (br s, 1H), 4.26 (br d, J=13.1 Hz, 1H), 4.04 (br d, J=7.5 Hz, 1H), 3.82 (br d, J=11.0 Hz, 1H), 3.70 (br d, J=9.0 Hz, 1H), 3.60-3.50 (m, 1H), 3.40 (br s, 1H), 3.32 (br s, 3H), 1.31 (br d, J=6.5 Hz, 3H)

### Example 24

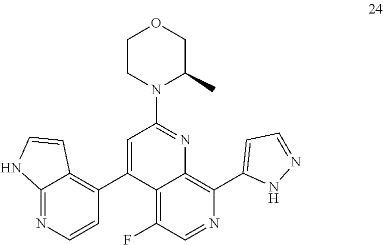 [SMILES: C[C@@H]1COCCN1c1cc(-c2ccnc3[nH]ccc23)c2c(F)cnc(-c3ccn[nH]3)c2n1]

Synthetic route:

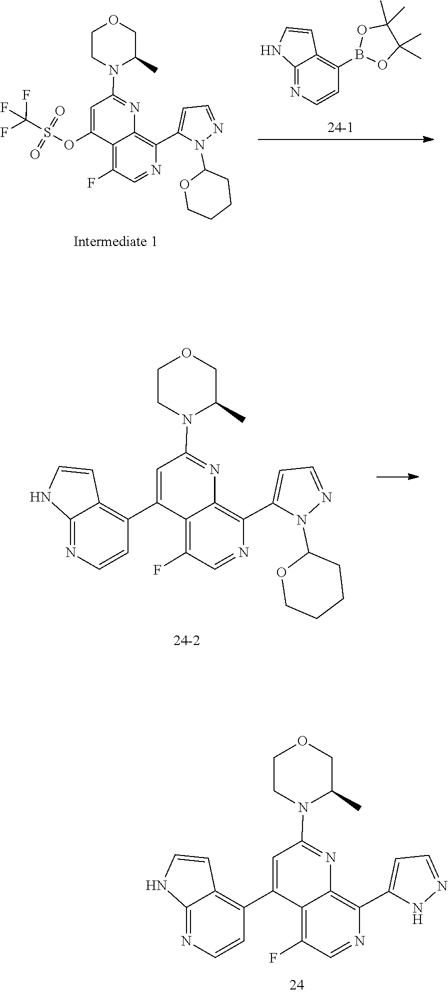 [SMILES: C[C@@H]1COCCN1c1cc(OS(=O)(=O)C(F)(F)F)c2c(F)cnc(-c3ccnn3C3CCCCO3)c2n1 ; CC1(C)OB(c2ccnc3[nH]ccc23)OC1(C)C ; C[C@@H]1COCCN1c1cc(-c2ccnc3[nH]ccc23)c2c(F)cnc(-c3ccnn3C3CCCCO3)c2n1 ; C[C@@H]1COCCN1c1cc(-c2ccnc3[nH]ccc23)c2c(F)cnc(-c3ccn[nH]3)c2n1]

Step 1: Synthesis of Compound 24-2

Compound 24-1 (53.70 mg, 219.98 μmol), intermediate 1 (120 mg, 219.98 μmol), 1,1-bis[(diphenylphosphino)ferrocene]palladium dichloride (13.41 mg, 18.33 μmol), and sodium carbonate (38.86 mg, 366.63 μmol) were mixed in dioxane (2 mL) and water (0.2 mL).

Nitrogen gas was bubbled into the system for 15 seconds, and then the mixture was stirred at 100° C. in microwave for 30 min. The reaction solution was filtered through diatomaceous earth, and the filtrate was rotary-evaporated under reduced pressure to afford a crude product.

The crude product was purified by column chromatography (eluent: tetrahydrofuran:petroleum ether=0˜25%˜50%) to afford compound 24-2.

MS m/z: 514.1 [M+H]⁺

Step 2: Synthesis of Compound 24

Compound 24-2 (70 mg, 136.30 μmol) was dissolved in 4M hydrochloric acid in dioxane (10 mL) and ethanol (2 mL). The reaction solution was stirred at 15° C. for 1 h, and then rotary-evaporated under reduced pressure to afford a crude product. The crude product was adjusted to a pH of 8 with saturated sodium bicarbonate solution. The mixture was extracted with dichloromethane (100 mL), washed with saturated brine (100 mL), dried over anhydrous sodium sulfate, and filtered to remove the desiccant. The filtrate was rotary evaporated under reduced pressure, then stirred with petroleum ether and ethyl acetate (petroleum ether:ethyl acetate=1:1, 15 mL), and filtered. The filter cake was dried by suction under reduced pressure to afford compound 24.

MS m/z: 430.1 [M+H]⁺

¹H NMR (400 MHz, DMSO-d₆) δ 11.86 (br s, 1H), 8.33 (d, J=5.0 Hz, 1H), 8.20 (d, J 2.0 Hz, 1H), 7.67 (d, J=1.5 Hz, 1H), 7.52-7.45 (m, 2H), 7.39-7.35 (m, 1H), 7.17 (d, J 5.0 Hz, 1H), 6.14 (dd, J=1.8, 3.3 Hz, 1H), 4.63 (br d, J=5.0 Hz, 1H), 4.24 (br d, J=12.5 Hz, 1H), 4.07-3.99 (m, 1H), 3.83-3.77 (m, 1H), 3.74-3.67 (m, 1H), 3.61-3.49 (m, 2H), 1.31 (br t, J=7.8 Hz, 3H)

### Example 25

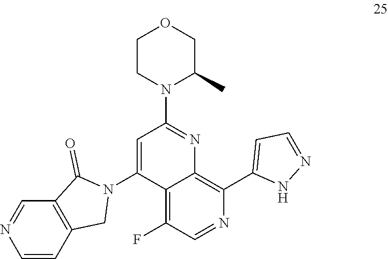 [SMILES: C[C@@H]1COCCN1c1cc(N2Cc3ccncc3C2=O)c2c(F)cnc(-c3ccn[nH]3)c2n1]

Synthetic route:

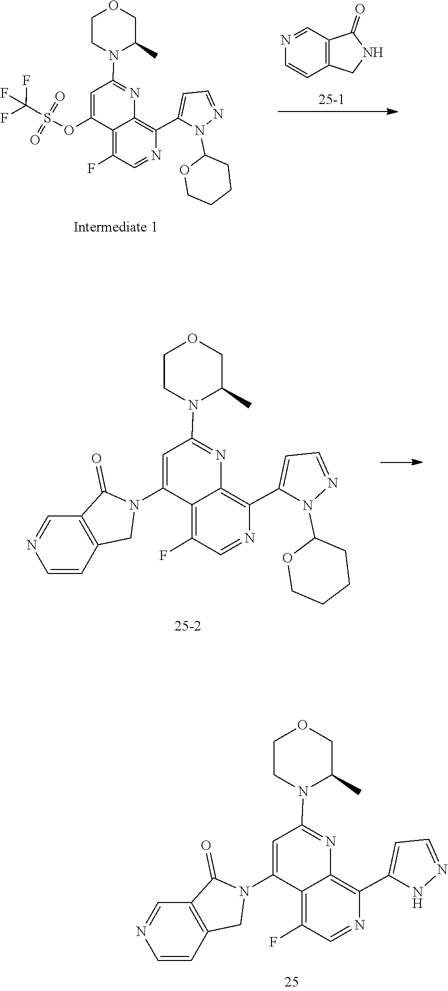 [SMILES: C[C@@H]1COCCN1c1cc(OS(=O)(=O)C(F)(F)F)c2c(F)cnc(-c3ccnn3C3CCCCO3)c2n1 ; O=C1NCc2ccncc21 ; C[C@@H]1COCCN1c1cc(N2Cc3ccncc3C2=O)c2c(F)cnc(-c3ccnn3C3CCCCO3)c2n1 ; C[C@@H]1COCCN1c1cc(N2Cc3ccncc3C2=O)c2c(F)cnc(-c3ccn[nH]3)c2n1]

Step 1: Synthesis of Compound 25-2

Compound 25-1 (20.19 mg, 150.50 μmol), intermediate 1 (63.15 mg, 115.77 μmol), methanesulfonato(2-dicyclohexylphosphino-3,6-dimethoxy-2,4,6-tri-i-propyl-1,1-biphenyl)(2-amino-1,1-biphenyl-2-yl)palladium(II) (10.49 mg), 2-(di-tert-butylphosphino)biphenyl (34.55 mg, 115.77 μmol), and cesium carbonate (113.16 mg, 347.31 μmol) were mixed in dioxane (2.5 mL). Nitrogen gas was bubbled into the system for 15 seconds, and then the mixture was stirred at 110° C. for 75 min. The reaction solution was filtered through diatomaceous earth, and the filtrate was rotary-evaporated under reduced pressure to afford a crude product. The crude product was purified by column chromatography (eluent: tetrahydrofuran:petroleum ether=0˜10%˜25%˜50%) to afford compound 25-2.

MS m/z: 530.1 [M+H]⁺

Step 2: Synthesis of Compound 25

Compound 25-2 (50 mg, 94.42 μmol) was dissolved in dichloromethane (2 mL) and trifluoroacetic acid (6 mL). The reaction solution was stirred at 15° C. for 12 h, and then adjusted to a pH of 8 with saturated sodium bicarbonate solution. The mixture was extracted with dichloromethane and methanol (dichloromethane:methanol=20:1, 100 mL), washed with saturated brine (50 mL), dried over anhydrous sodium sulfate, and filtered to remove the desiccant. The filtrate was rotary evaporated under reduced pressure to afford a crude product.

The crude product was purified by neutral preparative high performance liquid chromatography to afford compound 25.

MS m/z: 446.0 [M+H]⁺

¹H NMR (400 MHz, CHLOROFORM-d) δ 9.27 (s, 1H), 8.92 (d, J=5.5 Hz, 1H), 8.26 (d, J=2.5 Hz, 1H), 7.75 (d, J=1.5 Hz, 1H), 7.58 (d, J=5.0 Hz, 1H), 7.27 (s, 1H), 4.93 (s, 2H), 4.45 (br s, 1H), 4.25-4.14 (m, 1H), 4.05 (br d, J=11.5 Hz, 1H), 3.98-3.92 (m, 1H), 3.86 (dd, J 3.0, 11.5 Hz, 1H), 3.78-3.67 (m, 2H), 3.60 (dt, J=3.8, 12.4 Hz, 1H), 1.52 (d, J=7.0 Hz, 3H)

### Example 26

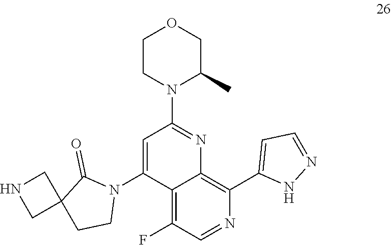 [SMILES: C[C@@H]1COCCN1c1cc(N2CCC3(CNC3)C2=O)c2c(F)cnc(-c3ccn[nH]3)c2n1]

Synthetic route:

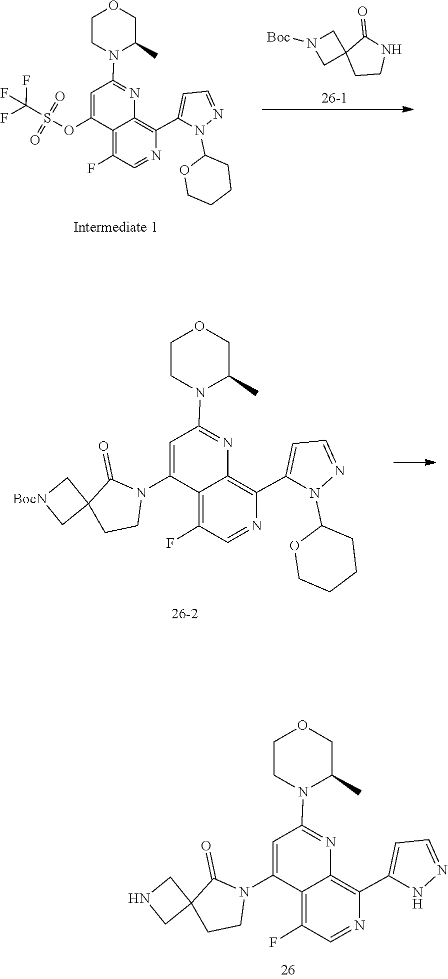 [SMILES: C[C@@H]1COCCN1c1cc(OS(=O)(=O)C(F)(F)F)c2c(F)cnc(-c3ccnn3C3CCCCO3)c2n1 ; CC(C)(C)OC(=O)N1CC2(CCNC2=O)C1 ; C[C@@H]1COCCN1c1cc(N2CCC3(CN(C(=O)OC(C)(C)C)C3)C2=O)c2c(F)cnc(-c3ccnn3C3CCCCO3)c2n1 ; C[C@@H]1COCCN1c1cc(N2CCC3(CNC3)C2=O)c2c(F)cnc(-c3ccn[nH]3)c2n1]

Step 1: Synthesis of Compound 26-2

Compound 26-1 (22.81 mg, 100.83 μmol), intermediate 1 (50 mg, 91.66 μmol), methanesulfonato(2-dicyclohexylphosphino-2,6-di-i-propoxy-1,1-biphenyl)(2-amino-1,1-bip henyl-2-yl)palladium(II) (7.67 mg, 9.17 μmol), 2-dicyclohexylphosphino-2,6-diisopropoxy-1,1-biphenyl (8.55 mg, 18.33 μmol), and cesium carbonate (89.59 mg, 274.98 μmol) were mixed in dioxane (2 mL). Nitrogen gas was bubbled into the system for 15 seconds, and then the mixture was stirred at 110° C. for 1 h. The reaction solution was filtered through diatomaceous earth, and the filtrate was rotary-evaporated under reduced pressure to afford a crude product. The crude product was purified by column chromatography (eluent: tetrahydrofuran:petroleum ether=0-50%) to afford compound 26-2.

MS m/z: 622.1 [M+H]⁺

Step 2: Synthesis of Compound 26

Compound 26-2 (80 mg, 128.68 μmol) was dissolved in dichloromethane (10 mL) and trifluoroacetic acid (1 mL). The reaction solution was stirred at 15° C. for 1 h, and then concentrated under reduced pressure to afford a crude product. The crude product was purified by acidic preparative high performance liquid chromatography to afford compound 26.

MS m/z: 438.1 [M+H]⁺

¹H NMR (400 MHz, ACETONITRILE-d₃) δ 8.77 (br s, 1H), 8.44 (br s, 1H), 7.96-7.84 (m, 2H), 7.42 (s, 1H), 7.24 (br s, 1H), 4.44-4.35 (m, 3H), 4.24-4.15 (m, 3H), 4.06-4.01 (m, 1H), 3.95-3.85 (m, 4H), 3.73-3.68 (m, 2H), 2.73 (t, J=6.8 Hz, 2H), 1.51 (d, J=6.5 Hz, 3H)

### Example 27

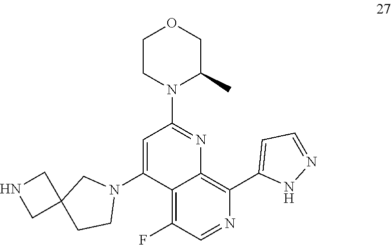 [SMILES: C[C@@H]1COCCN1c1cc(N2CCC3(CNC3)C2)c2c(F)cnc(-c3ccn[nH]3)c2n1]

Synthetic route:

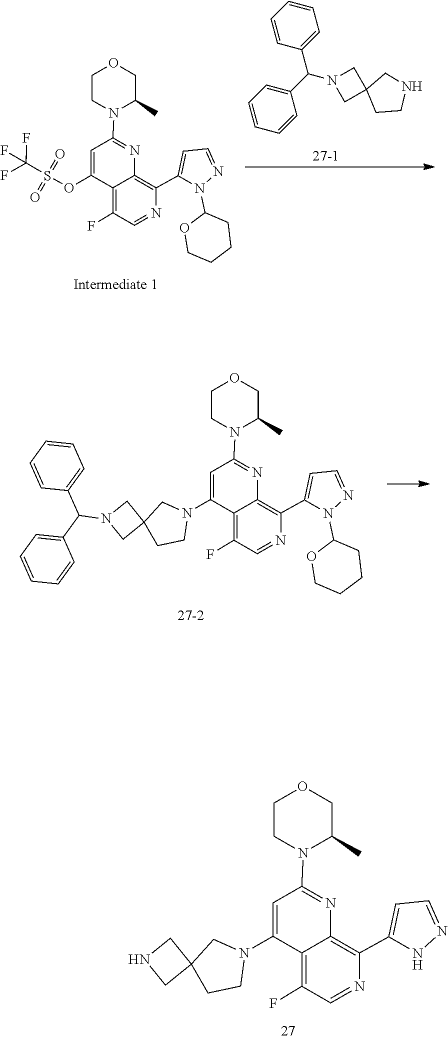 [SMILES: C[C@@H]1COCCN1c1cc(OS(=O)(=O)C(F)(F)F)c2c(F)cnc(-c3ccnn3C3CCCCO3)c2n1 ; c1ccc(C(c2ccccc2)N2CC3(CCNC3)C2)cc1 ; C[C@@H]1COCCN1c1cc(N2CCC3(C2)CN(C(c2ccccc2)c2ccccc2)C3)c2c(F)cnc(-c3ccnn3C3CCCCO3)c2n1 ; C[C@@H]1COCCN1c1cc(N2CCC3(CNC3)C2)c2c(F)cnc(-c3ccn[nH]3)c2n1]

Step 1: Synthesis of Compound 27˜2

Compound 27˜1 (30.62 mg, 109.99 μmol), intermediate 1 (50 mg, 91.66 μmol), methanesulfonato(2-dicyclohexylphosphino-2,6-di-i-propoxy-1,1-biphenyl)(2-amino-1,1-bip henyl-2-yl)palladium(II) (7.67 mg, 9.17 μmol), 2-dicyclohexylphosphino-2,6-diisopropoxy-1,1-biphenyl (8.55 mg, 18.33 μmol), and cesium carbonate (89.59 mg, 274.98 μmol) were mixed in dioxane (8 mL). Nitrogen gas was bubbled into the system for 15 seconds, and then the mixture was stirred at 110° C. for 1 h. The reaction solution was filtered through diatomaceous earth, and the filtrate was rotary-evaporated under reduced pressure to afford a crude product. The crude product was purified by column chromatography (eluent: tetrahydrofuran:petroleum ether=0˜25%˜50%) to afford compound 27-2.

MS m/z: 674.1 [M+H]⁺

Step 2: Synthesis of Compound 27

Compound 27-2 (120 mg, 178.09 μmol) was mixed in methanol (30 mL) and hydrochloric acid (0.4 mL, 12M). Palladium hydroxide (0.15 g) was added under nitrogen, and the mixture was stirred at 30° C. under hydrogen (50 Psi) atmosphere for 15 hours. The reaction solution was filtered through diatomaceous earth, and the filtrate was concentrated under reduced pressure to afford a crude product. The crude product was purified by preparative high performance liquid chromatography (hydrochloric acid) to afford compound 27.

MS m/z: 424.1 [M+H]⁺

¹H NMR (400 MHz, METHANOL-d4) δ 8.51 (d, J=3.0 Hz, 1H), 7.92 (d, J=2.5 Hz, 1H), 7.19 (d, J=2.5 Hz, 1H), 6.23 (br s, 1H), 4.40 (br s, 1H), 4.23 (br s, 2H), 4.15 (br d, J=6.5 Hz, 3H), 4.03 (br s, 2H), 3.96 (br d, J=12.5 Hz, 2H), 3.92-3.87 (m, 1H), 3.86-3.81 (m, 1H), 3.80 (br s, 2H), 3.70 (br d, J=12.5 Hz, 1H), 2.44 (br s, 2H), 1.57-1.51 (m, 3H)

### Example 28

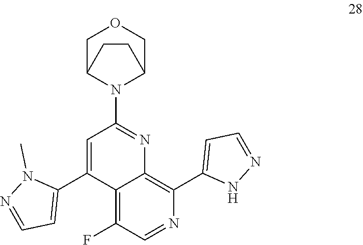 [SMILES: Cn1nccc1-c1cc(N2C3CCC2COC3)nc2c(-c3ccn[nH]3)ncc(F)c12]

Synthetic route:

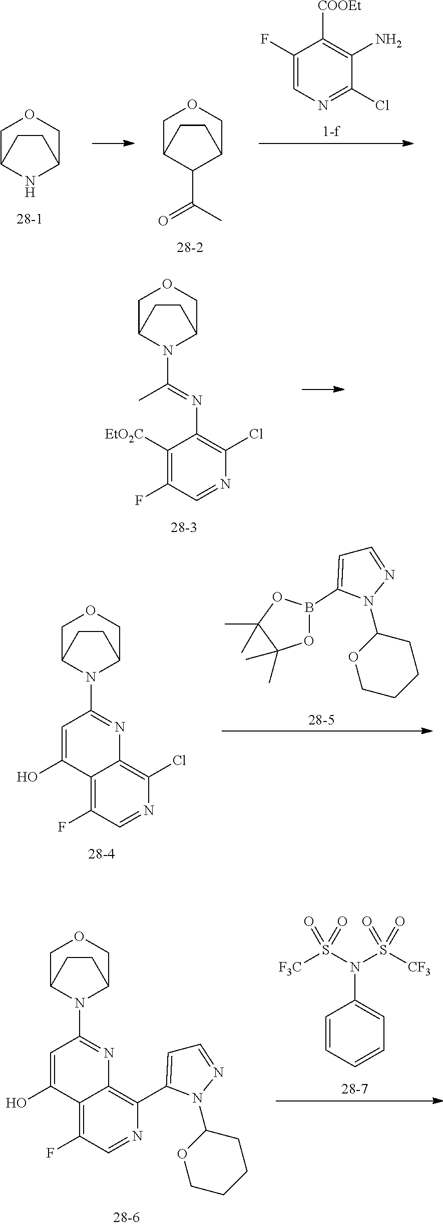

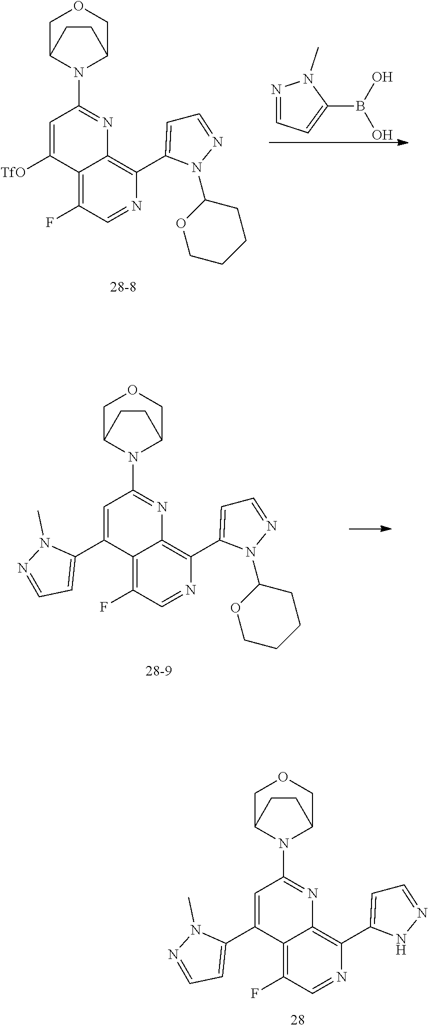

Step 1: Synthesis of Compound 28-2

Compound 28-1 (3 g, 20.05 mmol) was dissolved in dichloromethane (45 mL), and potassium carbonate (8.31 g, 60.15 mmol) was added. The mixture was stirred at 25° C. for 0.5 hours, and acetylchloride (3.15 g, 40.10 mmol, 2.86 mL) was added dropwise to the mixture. The mixture was stirred at 25° C. for another 10 hours. The reaction solution was filtered through diatomaceous earth, and the filtrate was concentrated under reduced pressure to afford 28-2.

MS m/z: 155.8 [M+H]⁺

Step 2: Synthesis of Compound 28-3

Compound 28-2 (3.55 g, 22.87 mmol) was dissolved in 1,2-dichloroethane (20 mL), and the mixture was cooled down to 0° C. Phosphorus oxychloride (7.89 g, 51.46 mmol, 4.78 mL) was slowly added dropwise. The mixture was stirred at 25° C. for 30 minutes, and then a solution of compound 1-f (2.5 g, 11.44 mmol) in 1,2-dichloroethane (10 mL) was added to the mixture. After the addition was completed, the mixture was heated to 80° C., and stirred for another 11 hours. The reaction solution was cooled down to room temperature, and then slowly added to water (70 mL) while stirring. The mixture was adjusted to a pH of 8 with 2 M sodium hydroxide solution, and extracted with dichloromethane (40 mL×3). The organic phase was dried over anhydrous sodium sulfate, and filtered. The filtrate was concentrated under reduced pressure to afford a crude product. The crude product was purified by column chromatography (eluent: ethyl acetate:petroleum ether=0˜100%) to afford compound 28-3.

MS m/z: 356.0 [M+H]⁺

Step 3: Synthesis of Compound 28-4

Compound 28-3 (800 mg, 2.25 mmol) was dissolved in N,N-dimethylformamide (15 mL), and the air in the reaction system was replaced 3 times with nitrogen. The mixture was cooled down to 0° C., and lithium bis(trimethylsilyl)amide (1 M, 10.68 mL) was added dropwise. The mixture was stirred at 0° C. for 2 hours, and saturated ammonium chloride solution (20 mL) was added to the reaction solution. The mixture was adjusted to a pH of 5-6 with 1 M hydrochloric acid solution, and extracted with dichloromethane (30 mL×3). The organic phase was washed with saturated brine, dried over anhydrous sodium sulfate, and filtered. The filtrate was concentrated under reduced pressure to afford compound 28-4.

MS m/z: 309.9 [M+H]⁺

Step 4: Synthesis of Compound 28-6

Compound 28-4 (650 mg, 2.10 mmol), 28-5 (875.62 mg, 3.15 mmol), and 1,1-bis[(diphenylphosphino)ferrocene]palladium dichloride (153.56 mg, 209.87 μmol) were dissolved in N,N-dimethylformamide (8 mL), and sodium carbonate solution (2 M, 2.10 mL) was added dropwise. Nitrogen gas was bubbled into the system for 15 seconds, and then the mixture was stirred at 110° C. in microwave for 1 h. Water (20 mL) was added to the reaction solution. The mixture was extracted with dichloromethane (10 mL×5). The organic phase was concentrated under reduced pressure to afford a crude product. The crude product was purified by column chromatography (eluent: tetrahydrofuran:petroleum ether=50˜70%) to afford compound 28-6.

MS m/z: 426.1 [M+H]⁺

Step 5: Synthesis of Compound 28-8

Compound 28-6 (240 mg, 564.10 μmol) was dissolved in dichloromethane (4 mL), and N,N-diisopropylethylamine (145.81 mg, 1.13 mmol, 196.51 μL) was added. The mixture was stirred for 30 minutes, and 28-7 (302.29 mg, 846.15 μmol) was added. The reaction solution was stirred at 25° C. for 1.5 h, and then concentrated under reduced pressure to afford a crude product. The crude product was purified by column chromatography (eluent: tetrahydrofuran:petroleum ether=20˜23%) to afford compound 28-8.

MS m/z: 558.0 [M+H]⁺

Step 6: Synthesis of Compound 28-9

Compound 28-8 (200 mg, 358.73 μmol), 1-methyl-TH-pyrazole-5-boronic acid (67.76 mg, 538.10 μmol), 1,1-bis[(diphenylphosphino)ferrocene]palladium dichloride (26.25 mg, 35.87 μmol), and sodium carbonate (76.04 mg, 717.47 μmol) were mixed in dioxane (2 mL) and water (0.2 mL). Nitrogen gas was bubbled into the system for 15 seconds, and then the mixture was stirred at 100° C. in microwave for 1 h. The reaction solution was filtered through diatomaceous earth, and the filtrate was concentrated under reduced pressure to afford a crude product. The crude product was purified by column chromatography (eluent: tetrahydrofuran:petroleum ether=35˜40%) to afford compound 28-9.

MS m/z: 490.1 [M+H]⁺

Step 7: Synthesis of Compound 28

Compound 28-9 (150 mg, 306.41 μmol) was dissolved in methanol (3 mL), and hydrochloric acid/methanol (3 mL) was added dropwise. The reaction solution was stirred at 25° C. for 2 hours, then concentrated under reduced pressure, and adjusted to a pH of 8 with saturated sodium carbonate solution. The mixture was extracted with dichloromethane (10 mL×3). The organic phase was concentrated under reduced pressure to afford a crude product. The crude product was purified by thin layer chromatography (eluent: methanol:dichloromethane=1:10) to afford compound 28.

MS m/z: 406.0 [M+H]⁺

¹H NMR (400 MHz, DMSO-d₆) δ 13.39 (br s, 1H), 8.27 (d, J=2.0 Hz, 1H), 7.63 (br s, 1H), 7.55 (d, J=2.0 Hz, 1H), 7.49 (s, 1H), 7.29 (br s, 1H), 6.48 (d, J=2.0 Hz, 1H), 4.83 (br s, 2H), 3.83-3.65 (m, 4H), 3.64 (s, 3H), 2.12-1.98 (m, 4H)

### Example 29

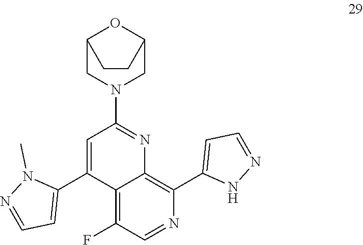 [SMILES: Cn1nccc1-c1cc(N2CC3CCC(C2)O3)nc2c(-c3ccn[nH]3)ncc(F)c12]

Synthetic route:

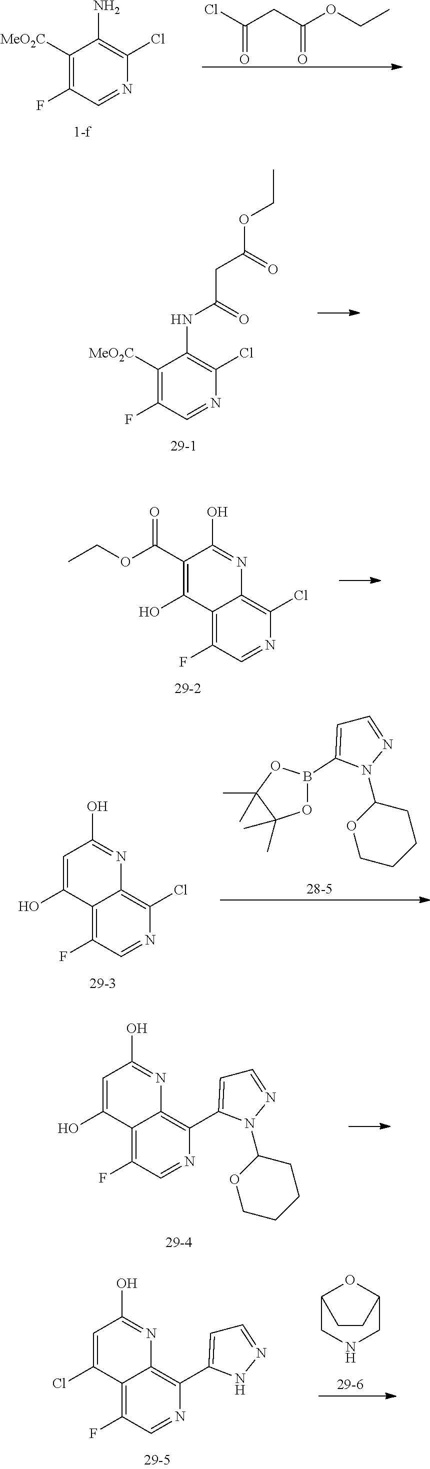

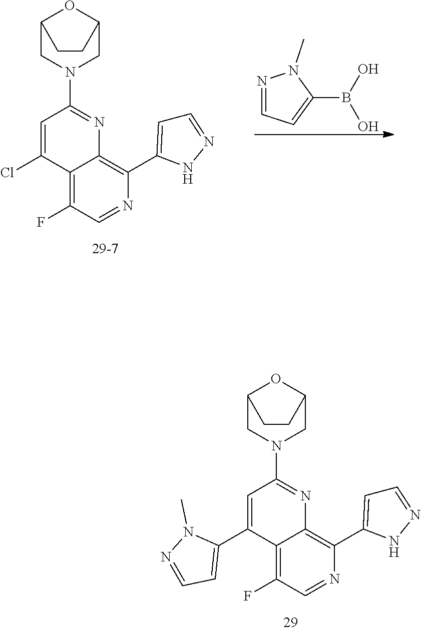

Step 1: Synthesis of Compound 29-1

Compound 1-f (5 g, 24.44 mmol) was dissolved in dichloromethane (50 mL), and the mixture was cooled down to 0° C. 4-Dimethylaminopyridine (298.58 mg, 2.44 mmol) and ethyl malonyl chloride (4.42 g, 29.33 mmol, 3.58 mL) were added, and the mixture was stirred at 25° C. for 16 hours. Water (20 mL) was added to the mixture, and the mixture was adjusted to a pH of 8 with saturated sodium carbonate solution. The mixture was extracted with dichloromethane (20 mL×4). The organic phase was concentrated under reduced pressure to afford a crude product. The crude product was purified by column chromatography (eluent: ethyl acetate:petroleum ether=0˜80%) to afford compound 29-1.

MS m/z: 318.9[M+H]⁺

Step 2: Synthesis of Compound 29-2

Sodium methoxide (2.71 g, 50.21 mmol) was dissolved in methanol (40 mL), and 29-1 (4 g, 12.55 mmol) was slowly added. The mixture was stirred at 25° C. for 1 hour, and the pH was adjusted to 3 with 1 M hydrochloric acid solution. Solids were precipitated. The mixture was filtered, and the filter cake was washed with water (30 mL×3). The solids were collected, and concentrated under reduced pressure to afford compound 29-2.

MS m/z: 286.9 [M+H]⁺

Step 3: Synthesis of Compound 29-3

Compound 29-2 (8.5 g, 29.65 mmol) was dissolved in hydrochloric acid (100 mL, 37% purity). The reaction solution was stirred at 72° C. for 16 hours, then cooled down to room temperature, and adjusted to a pH of 7 with 2M sodium hydroxide solution. The mixture was concentrated under reduced pressure to afford compound 29-3.

MS m/z: 214.7 [M+H]⁺

Step 4: Synthesis of Compound 29-4

Compound 29-3 (5 g, 23.30 mmol), 28-5 (9.72 g, 34.95 mmol), 1,1-bis[(diphenylphosphino)ferrocene]palladium dichloride dichloromethane (1.90 g, 2.33 mmol) and cesium carbonate (15.18 g, 46.60 mmol) were dissolved in dioxane (100 mL) and water (10 mL), and the air in the reaction system was replaced 3 times with nitrogen. The mixture was stirred at 90° C. for 12 hours. The reaction solution was cooled down to room temperature, and filtered through diatomaceous earth. The filtrate was concentrated under reduced pressure to afford a crude product of compound 29-4.

MS m/z: 330.8 [M+H]⁺

Step 5: Synthesis of Compound 29-5

Compound 29-4 (2.5 g, 10.15 mmol) was dissolved in phosphorus oxychloride (25 mL). The reaction solution was stirred at 90° C. for 2 hours, and then cooled down to room temperature. The reaction solution was slowly added to water (150 mL) while stirring, and the mixture was adjusted to a pH of 7 with 1M sodium hydroxide solution. The mixture was extracted with dichloromethane (200 mL×3). The organic phase was dried over anhydrous sodium sulfate, and filtered. The organic phase was concentrated under reduced pressure to afford a crude product. The crude product was purified by column chromatography (eluent: tetrahydrofuran:petroleum ether=20˜30%) to afford compound 29-5.

MS m/z: 282.8 [M+H]⁺

Step 6: Synthesis of Compound 29-7

Compound 29-5 (700 mg, 2.47 mmol) was dissolved in dioxane (20 mL), and 29-6 (480.95 mg, 3.21 mmol) and potassium carbonate (1.71 g, 12.36 mmol) were added. The mixture was stirred at 85° C. for 16 hours. The reaction solution was cooled down to room temperature, and filtered through diatomaceous earth. The filter cake was washed with dichloromethane (10 mL×3), then washed with acetonitrile (10 mL×3) and methanol (10 mL×3), and concentrated under reduced pressure to afford a crude product. The crude product was purified by acidic preparative high performance liquid chromatography to afford compound 29-7.

MS m/z: 359.8 [M+H]⁺

Step 7: Synthesis of Compound 29

Compound 29-7 (120 mg, 333.53 μmol) was dissolved in dioxane (2 mL) and water (0.2 mL), and 1-methyl-1H-pyrazole-5-boronic acid (126.00 mg, 1.0 mmol), bis(dibenzylideneacetone)palladium (19.18 mg, 33.35 μmol), 2-di-tert-butylphosphino-2′,4′,6′-triisopropylbiphenyl (31.80 mg, 66.71 μmol) and sodium carbonate (106.06 mg, 1.0 mmol) were added. Nitrogen gas was bubbled into the system for 15 seconds, and then the mixture was stirred at 120° C. in microwave for 1 h. The reaction solution was filtered through diatomaceous earth, and the filtrate was concentrated under reduced pressure to afford a crude product. The crude product was purified by acidic preparative high performance liquid chromatography to afford compound 29.

MS m/z: 406.0 [M+H]⁺

¹H NMR (400 MHz, DMSO-d₆) δ 8.30 (d, J=2.3 Hz, 1H), 7.69 (d, J=1.8 Hz, 1H), 7.55 (d, J=1.9 Hz, 1H), 7.48 (s, 1H), 7.35 (d, J=1.8 Hz, 1H), 6.47 (d, J=1.9 Hz, 1H), 4.55 (br s, 2H), 4.17 (br d, J=12.8 Hz, 2H), 3.62 (s, 3H), 3.33-3.04 (m, 2H), 1.91-1.78 (m, 4H)

### Assay Example 1: Assay of Cell Activity In Vitro

In this assay, the effect of the compounds on inhibiting cell proliferation was studied by detecting the effect of the compounds on the cell viability in vitro in the tumor cell line LoVo.

Cell viability detection using CellTiter-Glo luminescence assay

The following steps were performed according to the instructions of the Promega CellTiter-Glo Luminescent Cell Viability Detection Kit (Promega-G7573).

(1). A CellTiter-Glo buffer was thawed and allowed to stand to reach room temperature.

(2). A CellTiter-Glo substrate was allowed to stand to reach room temperature.

(3). The CellTiter-Glo buffer was added to the CellTiter-Glo substrate in a bottle to dissolve the substrate to formulate a CellTiter-Glo working solution.

(4). The working solution was vortexed slowly for fully dissolution.

(5). A cell culture plate was taken out and allowed to stand for 30 minutes to equilibrate to room temperature.

(6). 50 μL (equal to half the volume of cell culture solution in each well) of the CellTiter-Glo working solution was added into each well. The cell plate was wrapped with aluminum foil to protect the cell plate from light.

(7). The culture plate was shaken on an orbital shaker for 2 minutes to induce cell lysis.

(8). The culture plate was left at room temperature for 10 minutes to stabilize luminescent signals.

(9). The luminescent signals were detected on a plate reader (SpectraMax i3x of MolecμLar Devices).

Data Analysis

The inhibition rate (IR) of the assay compounds was calculated by the following formula:

IR (%)=(1−(RLU of compound−RLU of blank control)/(RLU of vehicle control−RLU of blank control))*100%.

The inhibition rates of different concentrations of compounds were calculated in Excel, and then GraphPad Prism software was used to draw inhibition curves and calculate relevant parameters, including the minimum inhibition rate, maximum inhibition rate, and IC₅₀.

The assay results are shown in Table 1:

TABLE 1
Results of in vitro LoVo cell proliferation inhibition assay

| Compound No. | IC₅₀ (nM) |
|---|---|
| Example 1 | 64 |
| Example 2 | 78 |
| Example 3 | 61 |
| Example 4 | 141 |
| Example 6 | 395 |
| Example 8 | 181 |
| Example 9 | 89 |
| Example 10 | 82 |
| Example 11 | 59 |
| Example 12 | 129 |
| Example 13 | 66 |
| Example 14 | 103 |
| Example 15 | 182 |
| Example 16 | 31 |
| Example 17 | 99 |
| Example 18 | 146 |
| Example 20 | 41 |
| Example 21 | 77 |
| Example 22 | 80 |
| Example 23 | 84 |
| Example 24 | 59 |
| Example 27 | 374 |
| Example 28 | 253 |
| Example 29 | 475 |

Assay conclusion: the compounds of the present disclosure have a good inhibitory effect on LoVo tumor cells with ATR signaling pathway mutation.

### Assay Example 2: Assay of CHK1(p-Ser345) In Vitro

The assay process was as follows:
1) When cells HT29 grew to about 80% confluence, the cells were digested and plated on a 96-well plate (80,000 cells/well), and 90 ul of cell suspension was added to each well; the cell plate was placed in a 5% carbon dioxide, 37° C. incubator overnight;
2) The next day, the supernatant was removed from the cell plate, and 90 ul of compounds with different concentrations were added; the plate was incubated at 37° C. for 1 hour, and then 10 ul of 7 uM 4NQO medium was added; the plate was incubated at 37° C. for 1 hour;
3) After the incubation was completed, the supernatant was removed, and 50 ul/well cell lysate was added; the plate was shaken and incubated at room temperature for 50 minutes;
4) After the incubation was completed, 8 ul/well cell lysate was transferred to a 384-well detection plate, and 5 ul of Acceptor was added; the plate was incubated at room temperature for 2 hours;
5) Then 2 ul of Donor was added, and the plate was incubated at room temperature overnight;
6) After the incubation was completed, alpha screen reading was performed.

The assay results are shown in Table 2:

TABLE 2
Assay results of inhibition of CHKI phosphorylation
at different concentrations

| | % Inhibition | |
|---|---|---|
| | 25 nM | 0.2 nM |
| Example 1 | 98.03 | 10.78 |
| Example 3 | 93.16 | 4.52 |
| Example 8 | 75.90 | 3.79 |
| Example 24 | 88.05 | 9.72 |
| Example 16 | 97.46 | 12.28 |
| Example 20 | 95.46 | 6.34 |
| Example 27 | 78.28 | 8.45 |
| Example 29 | 64.45 | 5.34 |

Assay conclusion: the compounds of the present disclosure have a good inhibitory effect on the phosphorylation of CHK11 protein, downstream of ATR signaling pathway.

### Assay Example 3: Assay of Pharmacokinetics In Vivo in Mice

The purpose of this assay is to study the pharmacokinetics of the compound of the present disclosure in the plasma of female Balb/c Nude mice after a single intravenous administration or a single oral administration.

In the intravenous group, plasma samples were collected at 9 time points of 5 minutes, 15 minutes, 30 minutes, 1 hour, 2 hours, 4 hours, 6 hours, 8 hours and 24 hours after administration; in the oral group, plasma samples were collected at 8 time points of 15 minutes, 30 minutes, 1 hour, 2 hours, 4 hours, 6 hours, 8 hours and 24 hours after administration; the samples were analyzed by LC-MS/MS for the plasma concentration data of the compound of the present disclosure, and the pharmacokinetic parameters, such as peak concentration, time to peak, clearance rate, half-life, maximum plasma drug concentration, area under the drug-time curve were calculated.

The assay results are shown in Table 3:

TABLE 3
Results of pharmacokinetic assay

| | Intravenous | | | |
|---|---|---|---|---|
| | injection (1 mg/kg) | | Oral (10 mg/kg) | |
| | Clearance | | Maximum | Area under the |
| | rate | | plasma drug | drug-time curve |
| | (mL/ | Half-life | concentration | AUC_{0-24 h} |
| | min/kg) | T_{1/2} (h) | Cₘₐₓ (nM) | (nM · hr) |
| Example 1 | 13.7 | 0.528 | 23641 | 41218 |

Conclusion: The compound of the present disclosure can significantly improve multiple indicators of pharmacokinetics in mice, in which the in vivo clearance rate and half-life of intravenous injection and the maximum blood drug concentration and area under the drug-time curve of oral administration have significant advantages.

### Assay Example 4: Assay of In Vivo Pharmacodynamics of the Compound on Subcutaneous Xenograft Tumor of Human Colorectal Cancer LoVo Cells in a BALB/c Nude Mouse Model

In this assay, the inhibitory effect of the compound of the present disclosure on the growth of subcutaneous xenograft tumor of human colorectal cancer LoVo cells was studied at an oral dosage of 40 mg/kg, twice a day, with 4 consecutive days of administration and 3 days of rest per week.

Assay method: The selected assay animals (provided by Shanghai Sippe-Bk Lab Animal Co., Ltd.) were BALB/c nude mice, 6-8 weeks old, weighing 18-22 grams.

Human colon cancer LoVo cells were cultured in a single layer in vitro with a culture condition of Ham's F-12 medium plus 10% fetal bovine serum, 100 U/mL penicillin, 100 μg/mL streptomycin and 2 mM glutamine, 37° C., 5% CO₂. Cells were routinely digested and passaged with trypsin-EDTA twice a week. When the cell saturation was 80%-90%, the cells were collected, counted and inoculated. 0.1 mL (10×10⁶ cells) of LoVo cells were subcutaneously inoculated on the right back of each nude mouse, and the mice were grouped and administered when the average tumor volume reached 146 mm³. Tumor diameters were measured twice a week with vernier calipers. The calculation formula of tumor volume was: V=0.5 a×b², wherein a and b represent the long and short diameters of tumor, respectively.

The antitumor efficacy of the compound was evaluated by TGI (%) or relative tumor proliferation rate T/C (%). TGI (%) reflected the inhibition rate of tumor growth. TGI (%) was calculated as follows: TGI (%)=[(1−(average tumor volume at the end of administration of a treatment group−average tumor volume at the beginning of administration of the treatment group))/(average tumor volume at the end of treatment of a solvent control group−average tumor volume at the beginning of treatment of the solvent control group)]×100%.

The assay result of the final 21 days of administration is shown in Table 4:

TABLE 4
Result of in vivo efficacy on mouse tumor

| Compound | TGI (%) |
|---|---|
| Example 1 | 92.4 |

Conclusion: the compound of the present disclosure can significantly improve the inhibitory effect on tumor growth in mice.

1. A compound of formula (II) or a pharmaceutically acceptable salt thereof, 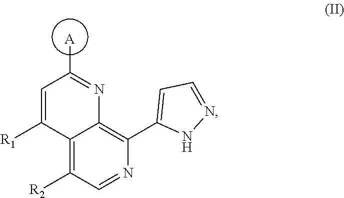
wherein
ring A is selected from 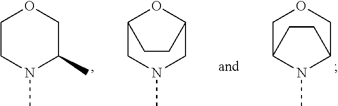
R₁ is H, D, F, Cl, Br, I, CN, C₁₋₃ alkyl, C₁₋₃ alkoxy, C₃₋₆ cycloalkyl, 5- to 10-membered heterocycloalkyl, 5- to 10-membered heteroaryl, phenyl, 5- to 6-membered heterocycloalkenyl, —C(═O)R₃, —C(═O)OR₃, —C(═O)NR₄R₅ or —NR₆C(═O)R₇, wherein the C₁₋₃ alkyl, C₁₋₃ alkoxy, C₃₋₆ cycloalkyl, 5- to 10-membered heterocycloalkyl, 5- to 10-membered heteroaryl, phenyl, and 5- to 6-membered heterocycloalkenyl are optionally substituted with 1, 2 or 3 Rₐ;
R₂ is F, Cl, Br and I;
R₃ is independently selected from H, D, C₁₋₃ alkyl, C₁₋₃ alkoxy and C₃₋₆ cycloalkyl;
R₄ and R₅ are independently selected from H, D, C₁₋₃ alkyl, C₁₋₃ alkoxy, and C₃₋₆ cycloalkyl, or
R₄ and R₅ are taken together with the N atom to which they are attached to form 5- to 6-membered heterocycloalkyl, wherein the 5- to 6-membered heterocycloalkyl is optionally substituted with 1, 2 or 3 Rₐ;
R₆ and R₇ are independently selected from H, D, C₁₋₃ alkyl, C₁₋₃ alkoxy, and C₃₋₆ cycloalkyl, or
R₆ and R₇ are taken together with the —N(C═O)— to which they are attached to form 5- to 10-membered heterocycloalkyl, wherein the 5- to 10-membered heterocycloalkyl is optionally substituted with 1, 2 or 3 Rₐ; and
Rₐ is H, D, F, Cl, Br, I, OH, NH₂, CN, COOH, —SO₂C₁₋₃ alkyl, C₁₋₃ alkyl or C₁₋₃ alkoxy.

2. The compound according to claim 1 or a pharmaceutically acceptable salt thereof, wherein Rₐ is independently selected from H, D, F, OH, CN, —OCH₃, —CH₃ and —SO₂CH₃.

3. The compound according to claim 1 or a pharmaceutically acceptable salt thereof, wherein R₂ is F.

4. The compound according to claim 1 or a pharmaceutically acceptable salt thereof, wherein R₁ is selected from 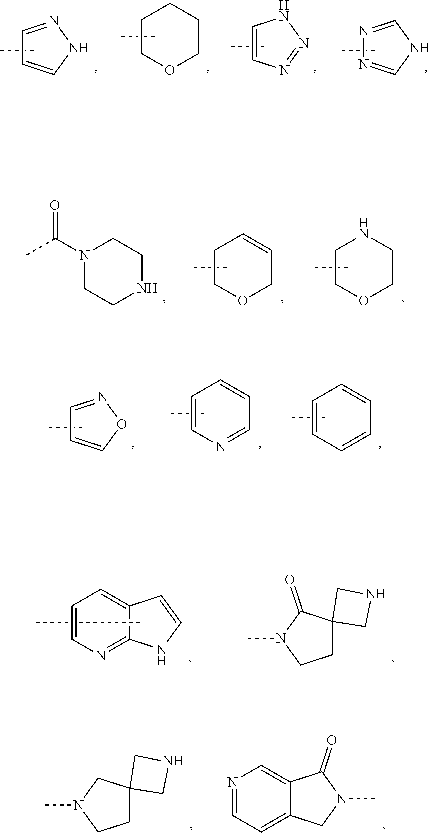
—OC₁₋₃ alkyl and C₃₋₆ cycloalkyl, wherein the 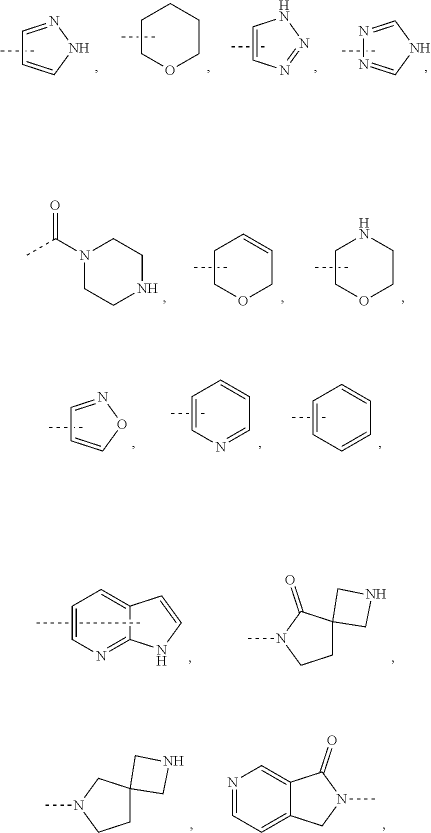
—OC₁₋₃ alkyl and C₃₋₆ cycloalkyl are optionally substituted with 1, 2 or 3 Rₐ.

5. The compound according to claim 4 or a pharmaceutically acceptable salt thereof,
wherein R₁ is selected from

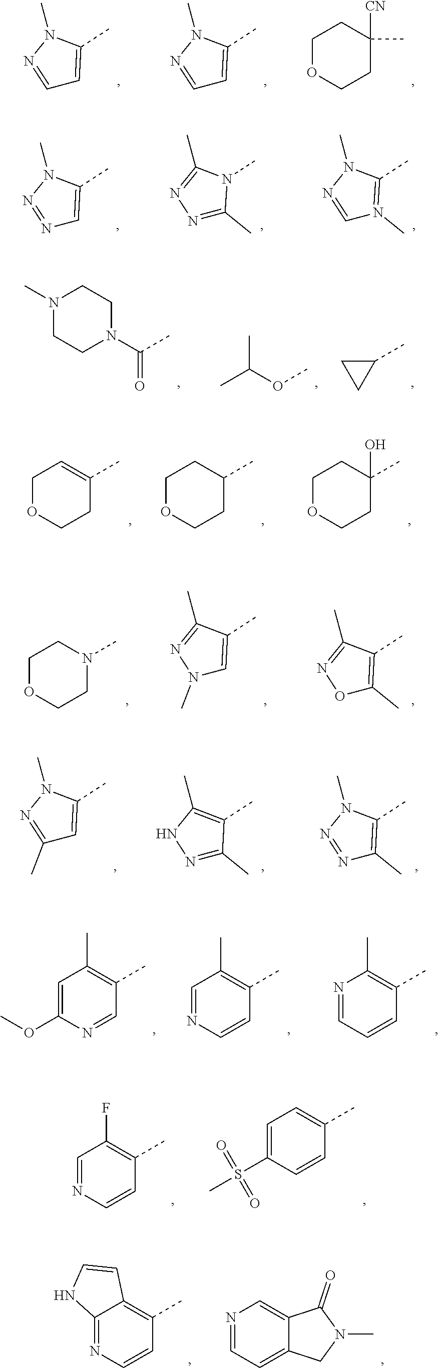

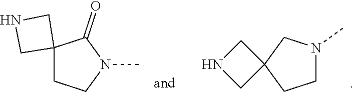

6. The compound according to claim 5 or a pharmaceutically acceptable salt thereof, wherein R₁ is

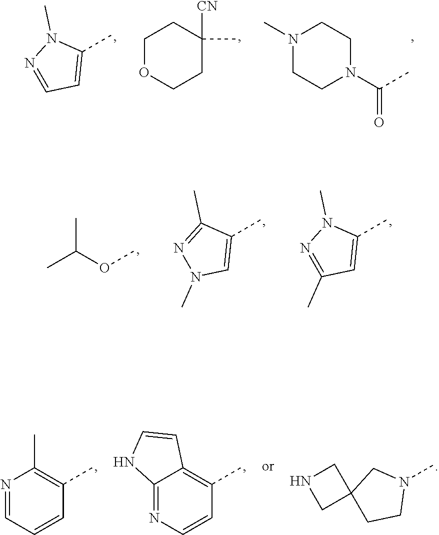

7. The compound according to claim 1, or a pharmaceutically acceptable salt thereof, wherein the compound is as shown in formulae (II-1), (II-2) and (II-3), 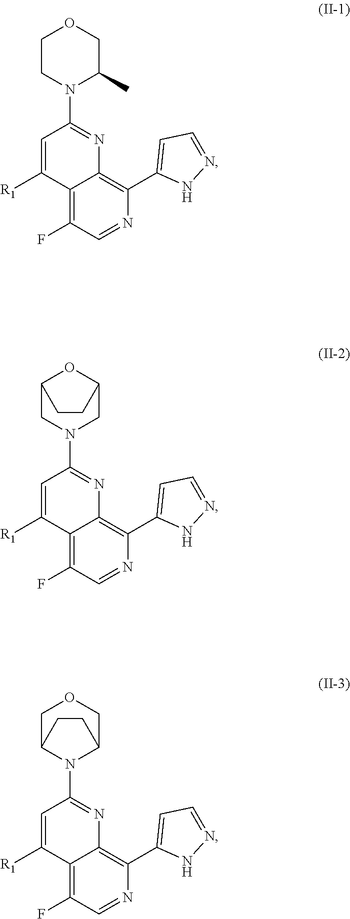
wherein R₁ is as defined in claim 1.

8. A compound or a pharmaceutically acceptable salt thereof, wherein the compound is selected from: 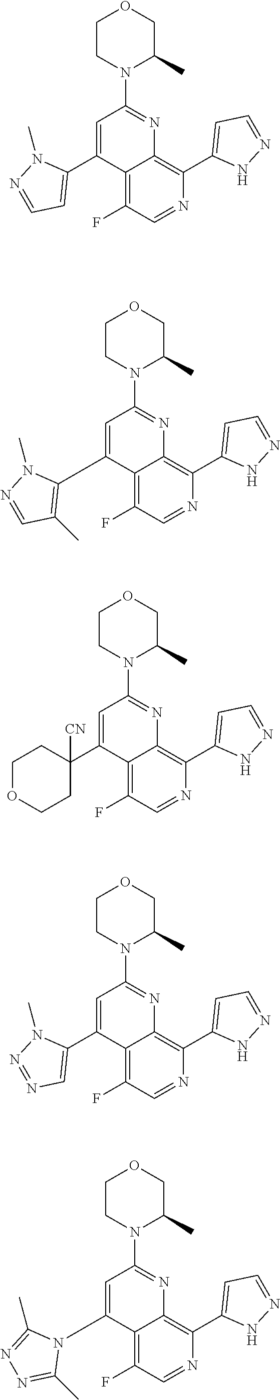 [SMILES: C[C@@H]1COCCN1c1cc(-c2ccnn2C)c2c(F)cnc(-c3ccn[nH]3)c2n1 ; Cc1cnn(C)c1-c1cc(N2CCOC[C@H]2C)nc2c(-c3ccn[nH]3)ncc(F)c12 ; C[C@@H]1COCCN1c1cc(C2(C#N)CCOCC2)c2c(F)cnc(-c3ccn[nH]3)c2n1 ; C[C@@H]1COCCN1c1cc(-c2cnnn2C)c2c(F)cnc(-c3ccn[nH]3)c2n1 ; Cc1nnc(C)n1-c1cc(N2CCOC[C@H]2C)nc2c(-c3ccn[nH]3)ncc(F)c12] 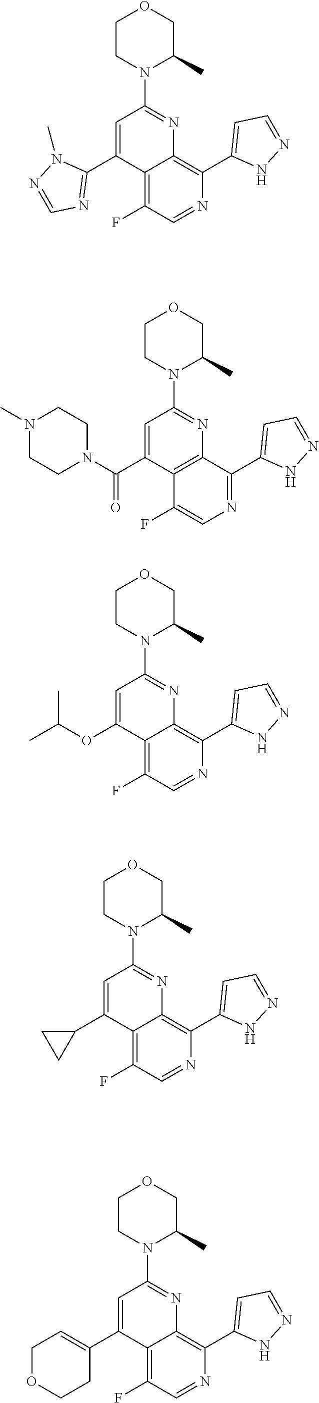 [SMILES: C[C@@H]1COCCN1c1cc(-c2ncnn2C)c2c(F)cnc(-c3ccn[nH]3)c2n1 ; C[C@@H]1COCCN1c1cc(C(=O)N2CCN(C)CC2)c2c(F)cnc(-c3ccn[nH]3)c2n1 ; CC(C)Oc1cc(N2CCOC[C@H]2C)nc2c(-c3ccn[nH]3)ncc(F)c12 ; C[C@@H]1COCCN1c1cc(C2CC2)c2c(F)cnc(-c3ccn[nH]3)c2n1 ; C[C@@H]1COCCN1c1cc(C2=CCOCC2)c2c(F)cnc(-c3ccn[nH]3)c2n1] 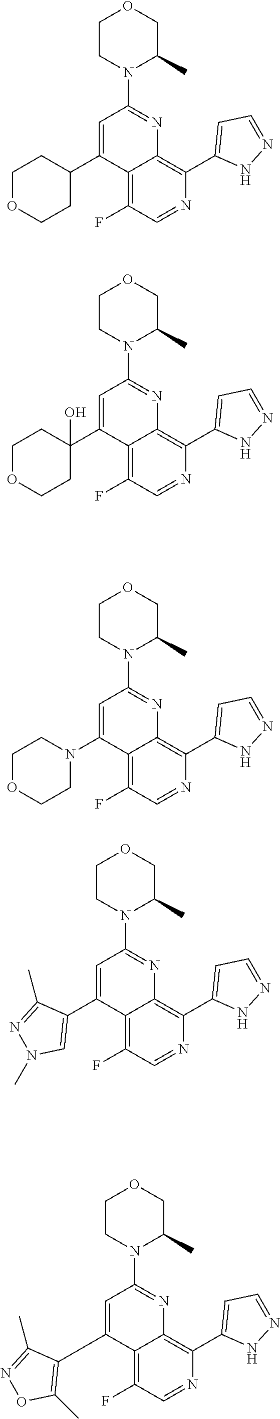 [SMILES: C[C@@H]1COCCN1c1cc(C2CCOCC2)c2c(F)cnc(-c3ccn[nH]3)c2n1 ; C[C@@H]1COCCN1c1cc(C2(O)CCOCC2)c2c(F)cnc(-c3ccn[nH]3)c2n1 ; C[C@@H]1COCCN1c1cc(N2CCOCC2)c2c(F)cnc(-c3ccn[nH]3)c2n1 ; Cc1nn(C)cc1-c1cc(N2CCOC[C@H]2C)nc2c(-c3ccn[nH]3)ncc(F)c12 ; Cc1noc(C)c1-c1cc(N2CCOC[C@H]2C)nc2c(-c3ccn[nH]3)ncc(F)c12] 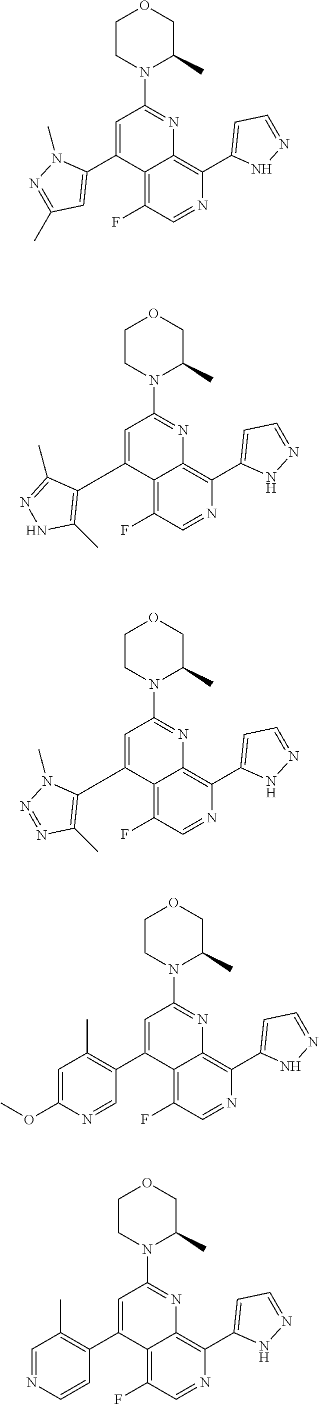 [SMILES: Cc1cc(-c2cc(N3CCOC[C@H]3C)nc3c(-c4ccn[nH]4)ncc(F)c23)n(C)n1 ; Cc1n[nH]c(C)c1-c1cc(N2CCOC[C@H]2C)nc2c(-c3ccn[nH]3)ncc(F)c12 ; Cc1nnn(C)c1-c1cc(N2CCOC[C@H]2C)nc2c(-c3ccn[nH]3)ncc(F)c12 ; COc1cc(C)c(-c2cc(N3CCOC[C@H]3C)nc3c(-c4ccn[nH]4)ncc(F)c23)cn1 ; Cc1cnccc1-c1cc(N2CCOC[C@H]2C)nc2c(-c3ccn[nH]3)ncc(F)c12] 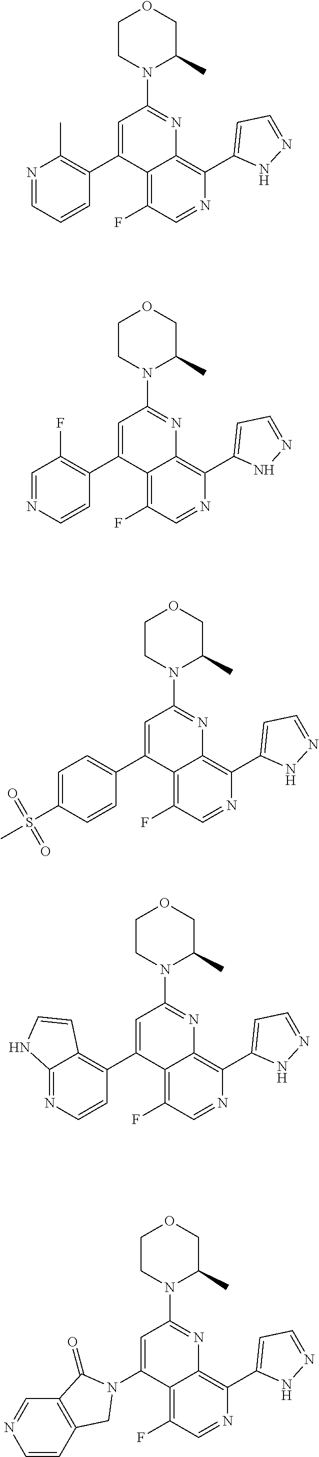 [SMILES: Cc1ncccc1-c1cc(N2CCOC[C@H]2C)nc2c(-c3ccn[nH]3)ncc(F)c12 ; C[C@@H]1COCCN1c1cc(-c2ccncc2F)c2c(F)cnc(-c3ccn[nH]3)c2n1 ; C[C@@H]1COCCN1c1cc(-c2ccc(S(C)(=O)=O)cc2)c2c(F)cnc(-c3ccn[nH]3)c2n1 ; C[C@@H]1COCCN1c1cc(-c2ccnc3[nH]ccc23)c2c(F)cnc(-c3ccn[nH]3)c2n1 ; C[C@@H]1COCCN1c1cc(N2Cc3ccncc3C2=O)c2c(F)cnc(-c3ccn[nH]3)c2n1] 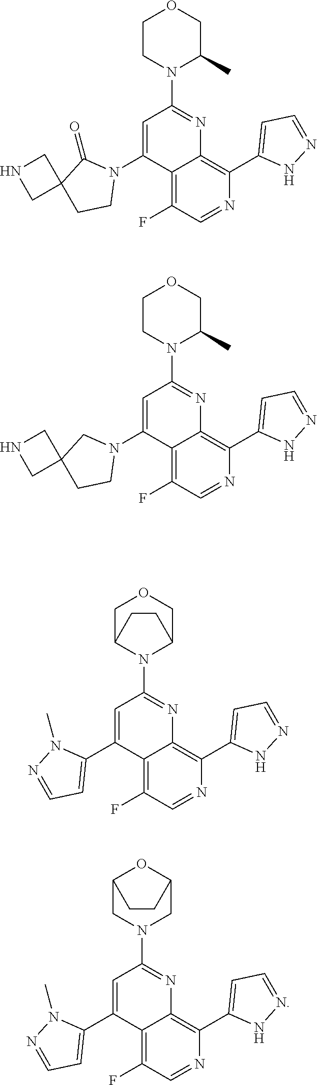 [SMILES: C[C@@H]1COCCN1c1cc(N2CCC3(CNC3)C2=O)c2c(F)cnc(-c3ccn[nH]3)c2n1 ; C[C@@H]1COCCN1c1cc(N2CCC3(CNC3)C2)c2c(F)cnc(-c3ccn[nH]3)c2n1 ; Cn1nccc1-c1cc(N2C3CCC2COC3)nc2c(-c3ccn[nH]3)ncc(F)c12 ; Cn1nccc1-c1cc(N2CC3CCC(C2)O3)nc2c(-c3ccn[nH]3)ncc(F)c12]
